(19) United States
(12) Patent Application Publication
(10) Pub. No.: US 2025/0313626 A1
(43) Pub. Date: Oct. 9, 2025

(54) ANTI-CD200R1 ANTIBODIES

(71) Applicant: Harbour BioMed (Shanghai) Co., Ltd., Shanghai (CN)

(72) Inventors: Musheng Bao, Shanghai (CN); Yuhua Wang, Shanghai (CN); Qiumei Du, Shanghai (CN); Hongjie Pan, Shanghai (CN); Geetika Bajpai, Shanghai (CN); Jinqiu He, Shanghai (CN); Yan Li, Shanghai (CN); Qingfang Chen, Shanghai (CN); Yongqiang Wang, Shanghai (CN); Xingxing Jia, Shanghai (CN); Fei Chen, Shanghai (CN); Youhong Wang, Shanghai (CN)

(21) Appl. No.: 18/866,805

(22) PCT Filed: May 18, 2023
(86) PCT No.: PCT/CN2023/094951

§ 371 Date: Nov. 18, 2024

(30) Foreign Application Priority Data
May 19, 2022 (WO) PCT/CN2022/093965

(51) Int. Cl. C07K 16/28; A61K 39/00; A61K 45/06; A61K 47/68; A61P 35/00

(52) U.S. Cl. CPC C07K 16/2803; A61K 45/06; A61K 47/6849; A61K 47/6879; A61P 35/00; A61K 2039/505; C07K 2317/31; C07K 2317/565

(57) ABSTRACT

Disclosed herein are antibodies that binds to CD200R1 and antigen-binding fragments thereof, as well as uses thereof, nucleic acids encoding the antibodies and antigen-binding fragments, vectors including the nucleic acids, and host cell including the nucleic acids or the vectors. Also disclosed are pharmaceutical compositions and conjugates including the antibodies, and therapeutic methods for using the antibodies.

### CROSS-REFERENCE TO RELATED APPLICATIONS

This application is a U.S. National Phase application of PCT Patent Application No.: PCT/CN2023/094951 filed May 18, 2023, which claims the priority of PCT Patent Application No.: PCT/CN2022/093965 filed on May 19, 2022, the entire contents of which are incorporated by reference for all purpose.

### REFERENCE TO SEQUENCE LISTING

The instant application contains a Sequence Listing which has been filed electronically in XML format and is hereby incorporated by reference in its entirety. Said XML copy, created on Nov. 23, 2023, is named PCTCN2023094951-seql .xml and is 245 KB in size.

### FIELD OF THE INVENTION

The invention relates to antibodies and antigen-binding fragments thereof that bind to CD200R1.

### BACKGROUND OF THE INVENTION

In normal tissues, CD200 is expressed on B cells, activated T cells, epithelial cells, neurons, and many other normal cell types. Further, CD200 has been shown to be highly expressed in different cancers, including non-small cell lung cancer, renal cell carcinoma, ovarian cancer, leukemia as well as many other cancers (Cancer Res 2020; 80(16 Suppl):Abstract nr 937; Cancer Immunol Immunother. 2008; 57(7):987-996; Proc Natl Acad Sci USA. 2006; 103(4):1041-1046). Anti-human antagonistic CD200 antibody, Samalizumab, has been used in Phase I clinical trial to treat chronic lymphocytic leukemia and multiple myeloma (J Immunother Cancer. 2019 Aug. 23; 7(1):227). Anti-CD200 antibody also demonstrated antitumor efficacy in pancreatic ductal adenocarcinoma model (J Immunother Cancer. 2020 June; 8(1):e000189).

CD200R1 is an Ig super family transmembrane glycoprotein expressed on the surface of myeloid cells and subsets of T cells (Immunity. 2000 August; 13(2):233-42; J Immunol 2003; 171:3034-3046). Human CD200R1 interacts with CD200 and viral CD200 homologues. The CD200-CD200R1 mediates suppressive signaling pathway and plays key roles in inhibiting the functions of T cells, myeloid cells, and NK cell (Int. J. Mol. Sci. 2021, 22(4), 1602). CD200R1 blockade has therapeutic potential in cancers. For example, in mouse studies, blocking the interaction of CD200 with CD200R1 by adenoviral delivery of soluble CD200R-Ig inhibited tumor growth (Mol Ther Oncolytics. 2021 Sep. 14; 23:138-150). When combined with thermal ablation treatment, anti-CD200R1 antibody greatly inhibited tumor growth (Med Sci Monit. 2019 Mar. 6; 25:1718-1728).

Comparing to anti-CD200 blocking antibody, CD200R1 is highly expressed on T cells and myeloid cells in tumor microenvironment. In addition, an anti-CD200R1 antagonistic antibody will block CD200-mediated as well as viral CD200-mediated inhibitory signaling pathway. An anti-CD200R1 antagonistic antibody will target CD200R1 overexpressing T cells and myeloid cells and restore their functions in tumor.

### SUMMARY OF THE INVENTION

The invention provides an antibody that specifically binds to CD200R1, or an antigen-binding fragment thereof.

The invention also provides a bi-specific antibody, comprising the antibody or antigen-binding fragment thereof of the invention and a second antigen binding region specifically binding to a tumor associated antigen or an immune checkpoint molecule.

The invention further provides a nucleic acid encoding the antibody or the bi-specific antibody of the invention.

The invention further provides a vector comprising a nucleic acid of the invention.

The invention further provides a host cell comprising a vector or a nucleic acid of the invention.

The invention further provides an antibody-drug conjugate (ADC), comprising the antibody or the antigen-binding fragment thereof of the invention or the bi-specific antibody of the invention.

The invention further provides a pharmaceutical composition comprising the antibody or the antigen-binding fragment thereof of the invention or the bispecific antibody of the invention, and optionally a pharmaceutically acceptable carrier or excipient.

The invention further provides an antibody of the invention for use in therapy.

The invention further provides an antibody of the invention for use in treating a cancer.

The invention further provides an antibody of the invention for use in activating T cells and/or myeloid cells in a cancer microenvironment.

The invention further provides a method for determining a subject suffering from a cancer or having a risk of developing a cancer, comprising:
(a) obtaining a biological sample from the subject,
(b) contacting the sample with an antibody or an antigen-binding fragment thereof of the invention, and
(c) detecting binding of the antibody to the sample,
wherein an increase in binding of the antibody to the sample as compared to binding of the antibody to a control sample indicates that the subject is suffering from a cancer or has a risk of developing a cancer. In some embodiments, the cancer is a cancer responsive to decreasing, inhibiting and/or blocking immune regulatory function or activity mediated by CD200R1.

The invention further provides a method for imaging a cancer in a subject, wherein the microenvironment of the cancer comprises T cells and/or myeloid cells on the surface of which CD200R1 is highly expressed, wherein the method comprises:
(a) administering the antibody or antigen binding fragment thereof of the invention to the subject, wherein the antibody is conjugated to a detectable marker, and
(b) detecting the presence of the marker.

In a first aspect, the invention provides an antibody that specifically binds to CD200R1, or an antigen binding fragment thereof, wherein the antibody comprises a light chain variable region (VL) and a heavy chain variable region (VH), and wherein
(1) the VH comprises the HCDRs 1-3 of a VH having the amino acid sequence set forth in SEQ ID NO: 117, and the VL comprises the LCDRs 1-3 of a VL having the amino acid sequence set forth in SEQ ID NO: 136; or
(2) the VH comprises the HCDRs 1-3 of a VH having the amino acid sequence set forth in SEQ ID NO: 111, and the VL comprises the LCDRs 1-3 of a VL having the amino acid sequence set forth in SEQ ID NO: 124; or
(3) the VH comprises the HCDRs 1-3 of a VH having the amino acid sequence set forth in SEQ ID NO: 117, and the VL comprises the LCDRs 1-3 of a VL having the amino acid sequence set forth in SEQ ID NO: 133; or
(4) the VH comprises the HCDRs 1-3 of a VH having the amino acid sequence set forth in SEQ ID NO: 118, and the VL comprises the LCDRs 1-3 of a VL having the amino acid sequence set forth in SEQ ID NO: 134; or
(5) the VH comprises the HCDRs 1-3 of a VH having the amino acid sequence set forth in SEQ ID NO: 119, and the VL comprises the LCDRs 1-3 of a VL having the amino acid sequence set forth in SEQ ID NO: 135; or
(6) the VH comprises the HCDRs 1-3 of a VH having the amino acid sequence set forth in SEQ ID NO: 120, and the VL comprises the LCDRs 1-3 of a VL having the amino acid sequence set forth in SEQ ID NO: 137; or
(7) the VH comprises the HCDRs 1-3 of a VH having the amino acid sequence set forth in SEQ ID NO: 108, and the VL comprises the LCDRs 1-3 of a VL having the amino acid sequence set forth in SEQ ID NO: 123; or
(8) the VH comprises the HCDRs 1-3 of a VH having the amino acid sequence set forth in SEQ ID NO: 109, and the VL comprises the LCDRs 1-3 of a VL having the amino acid sequence set forth in SEQ ID NO: 124; or
(9) the VH comprises the HCDRs 1-3 of a VH having the amino acid sequence set forth in SEQ ID NO: 110, and the VL comprises the LCDRs 1-3 of a VL having the amino acid sequence set forth in SEQ ID NO: 125; or
(10) the VH comprises the HCDRs 1-3 of a VH having the amino acid sequence set forth in SEQ ID NO: 114, and the VL comprises the LCDRs 1-3 of a VL having the amino acid sequence set forth in SEQ ID NO: 128; or
(11) the VH comprises the HCDRs 1-3 of a VH having the amino acid sequence set forth in SEQ ID NO: 115, and the VL comprises the LCDRs 1-3 of a VL having the amino acid sequence set forth in SEQ ID NO: 129; or
(12) the VH comprises the HCDRs 1-3 of a VH having the amino acid sequence set forth in SEQ ID NO: 116, and the VL comprises the LCDRs 1-3 of a VL having the amino acid sequence set forth in SEQ ID NO: 130; or
(13) the VH comprises the HCDRs 1-3 of a VH having the amino acid sequence set forth in SEQ ID NO: 115, and the VL comprises the LCDRs 1-3 of a VL having the amino acid sequence set forth in SEQ ID NO: 131; or
(14) the VH comprises the HCDRs 1-3 of a VH having the amino acid sequence set forth in SEQ ID NO: 116, and the VL comprises the LCDRs 1-3 of a VL having the amino acid sequence set forth in SEQ ID NO: 132.

In a second aspect, the invention provides an antibody that specifically binds to CD200R1, or an antigen binding fragment thereof, wherein the antibody comprises a light chain variable region (VL) and a heavy chain variable region (VH), and wherein
(1) the VL comprises LCDR1-3 having the amino acid sequences set forth in SEQ ID NOs: 67, 80, 99 respectively or LCDRs 1-3 having amino acid sequences each of which differ by one or two amino acid residues as the sequences set forth in SEQ ID NOs: 67, 80, 99 respectively, and the VH comprises HCDRs 1-3 having the amino acid sequences set forth in SEQ ID NOs: 11, 32, 46 respectively or HCDRs 1-3 having amino acid sequences each of which differ by one or two amino acid residues as the sequences set forth in SEQ ID NOs: 11, 32, 46 respectively; or
(2) the VL comprises LCDR1-3 having the amino acid sequences set forth in SEQ ID NOs: 62, 76, 92 respectively or LCDRs 1-3 having amino acid sequences each of which differ by one or two amino acid residues as the sequences set forth in SEQ ID NOs: 62, 76, 92 respectively, and the VH comprises HCDRs 1-3 having the amino acid sequences set forth in SEQ ID NOs: 11, 26, 46 respectively or HCDRs 1-3 having amino acid sequences each of which differ by one or two amino acid residues as the sequences set forth in SEQ ID NOs: 11, 26, 46 respectively; or
(3) the VL comprises LCDR1-3 having the amino acid sequences set forth in SEQ ID NOs: 66, 80, 92 respectively or LCDRs 1-3 having amino acid sequences each of which differ by one or two amino acid residues as the sequences set forth in SEQ ID NOs: 66, 80, 92 respectively, and the VH comprises HCDRs 1-3 having the amino acid sequences set forth in SEQ ID NOs: 11, 32, 46 respectively or HCDRs 1-3 having amino acid sequences each of which differ by one or two amino acid residues as the sequences set forth in SEQ ID NOs: 11, 32, 46 respectively; or
(4) the VL comprises LCDR1-3 having the amino acid sequences set forth in SEQ ID NOs: 66, 81, 92 respectively or LCDRs 1-3 having amino acid sequences each of which differ by one or two amino acid residues as the sequences set forth in SEQ ID NOs: 66, 81, 92 respectively, and the VH comprises HCDRs 1-3 having the amino acid sequences set forth in SEQ ID NOs: 11, 33, 46 respectively or HCDRs 1-3 having amino acid sequences each of which differ by one or two amino acid residues as the sequences set forth in SEQ ID NOs: 11, 33, 46 respectively; or
(5) the VL comprises LCDR1-3 having the amino acid sequences set forth in SEQ ID NOs: 67, 76, 99 respectively or LCDRs 1-3 having amino acid sequences each of which differ by one or two amino acid residues as the sequences set forth in SEQ ID NOs: 67, 76, 99 respectively, and the VH comprises HCDRs 1-3 having the amino acid sequences set forth in SEQ ID NOs: 14, 32, 46 respectively or HCDRs 1-3 having amino acid sequences each of which differ by one or two amino acid residues as the sequences set forth in SEQ ID NOs: 14, 32, 46 respectively; or
(6) the VL comprises LCDR1-3 having the amino acid sequences set forth in SEQ ID NOs: 66, 80, 100 respectively or LCDRs 1-3 having amino acid sequences each of which differ by one or two amino acid residues as the sequences set forth in SEQ ID NOs: 66, 80, 100 respectively, and the VH comprises HCDRs 1-3 having the amino acid sequences set forth in SEQ ID NOs: 14, 33, 46 respectively or HCDRs 1-3 having amino acid sequences each of which differ by one or two amino acid residues as the sequences set forth in SEQ ID NOs: 14, 33, 46 respectively
(7) the VL comprises LCDRs 1-3 having the amino acid sequences set forth in SEQ ID NOs: 62, 76, 91 respectively or LCDRs 1-3 having amino acid sequences each of which differ by one or two amino acid residues as the sequences set forth in SEQ ID NOs: 62, 76, 91 respectively, and the VH comprises HCDRs 1-3 having the amino acid sequences set forth in SEQ ID NOs: 10, 25, 44 respectively or HCDRs 1-3 having amino acid sequences each of which differ by one or two amino acid residues as the sequences set forth in SEQ ID NOs: 10, 25, 44 respectively; or
(8) the VL comprises LCDR1-3 having the amino acid sequences set forth in SEQ ID NOs: 62, 76, 92 respectively or LCDRs 1-3 having amino acid sequences each of which differ by one or two amino acid residues as the sequences set forth in SEQ ID NOs: 62, 76, 92 respectively, and the VH comprises HCDRs 1-3 having the amino acid sequences set forth in SEQ ID NOs: 11, 26, 45 respectively or HCDRs 1-3 having amino acid sequences each of which differ by one or two amino acid residues as the sequences set forth in SEQ ID NOs: 11, 26, 45 respectively; or
(9) the VL comprises LCDR1-3 having the amino acid sequences set forth in SEQ ID NOs: 62, 76, 93 respectively or LCDRs 1-3 having amino acid sequences each of which differ by one or two amino acid residues as the sequences set forth in SEQ ID NOs: 62, 76, 93 respectively, and the VH comprises HCDRs 1-3 having the amino acid sequences set forth in SEQ ID NOs: 11, 25, 45 respectively or HCDRs 1-3 having amino acid sequences each of which differ by one or two amino acid residues as the sequences set forth in SEQ ID NOs: 11, 25, 45 respectively; or
(10) the VL comprises LCDR1-3 having the amino acid sequences set forth in SEQ ID NOs: 65, 79, 96 respectively or LCDRs 1-3 having amino acid sequences each of which differ by one or two amino acid residues as the sequences set forth in SEQ ID NOs: 65, 79, 96 respectively, and the VH comprises HCDRs 1-3 having the amino acid sequences set forth in SEQ ID NOs: 11, 29, 49 respectively or HCDRs 1-3 having amino acid sequences each of which differ by one or two amino acid residues as the sequences set forth in SEQ ID NOs: 11, 29, 49 respectively; or
(11) the VL comprises LCDR1-3 having the amino acid sequences set forth in SEQ ID NOs: 65, 79, 97 respectively or LCDRs 1-3 having amino acid sequences each of which differ by one or two amino acid residues as the sequences set forth in SEQ ID NOs: 65, 79, 97 respectively, and the VH comprises HCDRs 1-3 having the amino acid sequences set forth in SEQ ID NOs: 11, 30, 49 respectively or HCDRs 1-3 having amino acid sequences each of which differ by one or two amino acid residues as the sequences set forth in SEQ ID NOs: 11, 30, 49 respectively; or
(12) the VL comprises LCDR1-3 having the amino acid sequences set forth in SEQ ID NOs: 65, 79, 98 respectively or LCDRs 1-3 having amino acid sequences each of which differ by one or two amino acid residues as the sequences set forth in SEQ ID NOs: 65, 79, 98 respectively, and the VH comprises HCDRs 1-3 having the amino acid sequences set forth in SEQ ID NOs: 11, 31, 50 respectively or HCDRs 1-3 having amino acid sequences each of which differ by one or two amino acid residues as the sequences set forth in SEQ ID NOs: 11, 31, 50 respectively; or
(13) the VL comprises LCDR1-3 having the amino acid sequences set forth in SEQ ID NOs: 65, 79, 97 respectively or LCDRs 1-3 having amino acid sequences each of which differ by one or two amino acid residues as the sequences set forth in SEQ ID NOs: 65, 79, 97 respectively, and the VH comprises HCDRs 1-3 having the amino acid sequences set forth in SEQ ID NOs: 11, 30, 49 respectively or HCDRs 1-3 having amino acid sequences each of which differ by one or two amino acid residues as the sequences set forth in SEQ ID NOs: 11, 30, 49 respectively; or
(14) the VL comprises LCDR1-3 having the amino acid sequences set forth in SEQ ID NOs: 65, 79, 98 respectively or LCDRs 1-3 having amino acid sequences each of which differ by one or two amino acid residues as the sequences set forth in SEQ ID NOs: 65, 79, 98 respectively, and the VH comprises HCDRs 1-3 having the amino acid sequences set forth in SEQ ID NOs: 11, 31, 50 respectively or HCDRs 1-3 having amino acid sequences each of which differ by one or two amino acid residues as the sequences set forth in SEQ ID NOs: 11, 31, 50 respectively.

In some embodiments of the first or second aspect of the invention, the invention provides an antibody or antigen-binding fragment thereof that specifically binds to CD200R1, or an antigen binding fragment thereof, wherein
(1) the VL comprises the amino acid sequence having at least 80%, at least 85%, at least 90%, at least 95%, at least 98%, at least 99%, or 100% sequence identity to SEQ ID No:136, and the VH comprises the amino acid sequence having at least 80%, at least 85%, at least 90%, at least 95%, at least 98%, at least 99%, or 100% sequence identity to SEQ ID No:117; or
(2) the VL comprises the amino acid sequence having at least 80%, at least 85%, at least 90%, at least 95%, at least 98%, at least 99%, or 100% sequence identity to SEQ ID No:124, and the VH comprises the amino acid sequence having at least 80%, at least 85%, at least 90%, at least 95%, at least 98%, at least 99%, or 100% sequence identity to SEQ ID No:111; or
(3) the VL comprises the amino acid sequence having at least 80%, at least 85%, at least 90%, at least 95%, at least 98%, at least 99%, or 100% sequence identity to SEQ ID No:133, and the VH comprises the amino acid sequence having at least 80%, at least 85%, at least 90%, at least 95%, at least 98%, at least 99%, or 100% sequence identity to SEQ ID No:117; or
(4) the VL comprises the amino acid sequence having at least 80%, at least 85%, at least 90%, at least 95%, at least 98%, at least 99%, or 100% sequence identity to SEQ ID No:134, and the VH comprises the amino acid sequence having at least 80%, at least 85%, at least 90%, at least 95%, at least 98%, at least 99%, or 100% sequence identity to SEQ ID No:118; or
(5) the VL comprises the amino acid sequence having at least 80%, at least 85%, at least 90%, at least 95%, at least 98%, at least 99%, or 100% sequence identity to SEQ ID No:135, and the VH comprises the amino acid sequence having at least 80%, at least 85%, at least 90%, at least 95%, at least 98%, at least 99%, or 100% sequence identity to SEQ ID No:119; or
(6) the VL comprises the amino acid sequence having at least 80%, at least 85%, at least 90%, at least 95%, at least 98%, at least 99%, or 100% sequence identity to SEQ ID No 137, and the VH comprises the amino acid sequence having at least 80%, at least 85%, at least 90%, at least 95%, at least 98%, at least 99%, or 100% sequence identity to SEQ ID No 120; or
(7) the VL comprises the amino acid sequence having at least 80%, at least 85%, at least 90%, at least 95%, at least 98%, at least 99%, or 100% sequence identity to SEQ ID No:123, and the VH comprises the amino acid sequence having at least 80%, at least 85%, at least 90%, at least 95%, at least 98%, at least 99%, or 100% sequence identity to SEQ ID No:108; or
(8) the VL comprises the amino acid sequence having at least 80%, at least 85%, at least 90%, at least 95%, at least 98%, at least 99%, or 100% sequence identity to SEQ ID No:124, and the VH comprises the amino acid sequence having at least 80%, at least 85%, at least 90%, at least 95%, at least 98%, at least 99%, or 100% sequence identity to SEQ ID No:109; or
(9) the VL comprises the amino acid sequence having at least 80%, at least 85%, at least 90%, at least 95%, at least 98%, at least 99%, or 100% sequence identity to SEQ ID No:125, and the VH comprises the amino acid sequence having at least 80%, at least 85%, at least 90%, at least 95%, at least 98%, at least 99%, or 100% sequence identity to SEQ ID No:110; or
(10) the VL comprises the amino acid sequence having at least 80%, at least 85%, at least 90%, at least 95%, at least 98%, at least 99%, or 100% sequence identity to SEQ ID No:128, and the VH comprises the amino acid sequence having at least 80%, at least 85%, at least 90%, at least 95%, at least 98%, at least 99%, or 100% sequence identity to SEQ ID No:114; or
(11) the VL comprises the amino acid sequence having at least 80%, at least 85%, at least 90%, at least 95%, at least 98%, at least 99%, or 100% sequence identity to SEQ ID No:129, and the VH comprises the amino acid sequence having at least 80%, at least 85%, at least 90%, at least 95%, at least 98%, at least 99%, or 100% sequence identity to SEQ ID No:115; or
(12) the VL comprises the amino acid sequence having at least 80%, at least 85%, at least 90%, at least 95%, at least 98%, at least 99%, or 100% sequence identity to SEQ ID No:130, and the VH comprises the amino acid sequence having at least 80%, at least 85%, at least 90%, at least 95%, at least 98%, at least 99%, or 100% sequence identity to SEQ ID No:116; or
(13) the VL comprises the amino acid sequence having at least 80%, at least 85%, at least 90%, at least 95%, at least 98%, at least 99%, or 100% sequence identity to SEQ ID No:131, and the VH comprises the amino acid sequence having at least 80%, at least 85%, at least 90%, at least 95%, at least 98%, at least 99%, or 100% sequence identity to SEQ ID No:115; or
(14) the VL comprises the amino acid sequence having at least 80%, at least 85%, at least 90%, at least 95%, at least 98%, at least 99%, or 100% sequence identity to SEQ ID No:132, and the VH comprises the amino acid sequence having at least 80%, at least 85%, at least 90%, at least 95%, at least 98%, at least 99%, or 100% sequence identity to SEQ ID No:116.

In some embodiments of the first or second aspect of the invention, the invention provides an antibody or antigen-binding fragment thereof that specifically binds to CD200R1, or an antigen binding fragment thereof, wherein the antibody or antigen-binding fragment comprises a heavy chain (HC) and a light chain (LC), and wherein
(1) the LC comprises the amino acid sequence having at least 80%, at least 85%, at least 90%, at least 95%, at least 98%, at least 99%, or 100% sequence identity to SEQ ID No: 170, and the HC comprises the amino acid sequence having at least 80%, at least 85%, at least 90%, at least 95%, at least 98%, at least 99%, or 100% sequence identity to SEQ ID No: 151; or
(2) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 143; or
(3) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 147; or
(4) the LC comprises the amino acid sequence having at least 80%, at least 85%, at least 90%, at least 95%, at least 98%, at least 99%, or 100% sequence identity to SEQ ID No: 167, and the HC comprises the amino acid sequence having at least 80%, at least 85%, at least 90%, at least 95%, at least 98%, at least 99%, or 100% sequence identity to SEQ ID No: 151; or
(5) the LC comprises the amino acid sequence having at least 80%, at least 85%, at least 90%, at least 95%, at least 98%, at least 99%, or 100% sequence identity to SEQ ID No: 168, and the HC comprises the amino acid sequence having at least 80%, at least 85%, at least 90%, at least 95%, at least 98%, at least 99%, or 100% sequence identity to SEQ ID No: 152; or
(6) the LC comprises the amino acid sequence having at least 80%, at least 85%, at least 90%, at least 95%, at least 98%, at least 99%, or 100% sequence identity to SEQ ID No: 169, and the HC comprises the amino acid sequence having at least 80%, at least 85%, at least 90%, at least 95%, at least 98%, at least 99%, or 100% sequence identity to SEQ ID No: 153;
(7) the LC comprises the amino acid sequence having at least 80%, at least 85%, at least 90%, at least 95%, at least 98%, at least 99%, or 100% sequence identity to SEQ ID No: 171, and the HC comprises the amino acid sequence having at least 80%, at least 85%, at least 90%, at least 95%, at least 98%, at least 99%, or 100% sequence identity to SEQ ID No: 154; or
(8) the LC comprises the amino acid sequence having at least 80%, at least 85%, at least 90%, at least 95%, at least 98%, at least 99%, or 100% sequence identity to SEQ ID No: 157, and the HC comprises the amino acid sequence having at least 80%, at least 85%, at least 90%, at least 95%, at least 98%, at least 99%, or 100% sequence identity to SEQ ID No: 140; or
(9) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 141; or
(10) the LC comprises the amino acid sequence having at least 80%, at least 85%, at least 90%, at least 95%, at least 98%, at least 99%, or 100% sequence identity to SEQ ID No: 159, and the HC comprises the amino acid sequence having at least 80%, at least 85%, at least 90%, at least 95%, at least 98%, at least 99%, or 100% sequence identity to SEQ ID No: 142; or
(11) the LC comprises the amino acid sequence having at least 80%, at least 85%, at least 90%, at least 95%, at least 98%, at least 99%, or 100% sequence identity to SEQ ID No: 162, and the HC comprises the amino acid sequence having at least 80%, at least 85%, at least 90%, at least 95%, at least 98%, at least 99%, or 100% sequence identity to SEQ ID No: 146; or
(12) the LC comprises the amino acid sequence having at least 80%, at least 85%, at least 90%, at least 95%, at least 98%, at least 99%, or 100% sequence identity to SEQ ID No: 162, and the HC comprises the amino acid sequence having at least 80%, at least 85%, at least 90%, at least 95%, at least 98%, at least 99%, or 100% sequence identity to SEQ ID No: 148; or
(13) the LC comprises the amino acid sequence having at least 80%, at least 85%, at least 90%, at least 95%, at least 98%, at least 99%, or 100% sequence identity to SEQ ID No: 163, and the HC comprises the amino acid sequence having at least 80%, at least 85%, at least 90%, at least 95%, at least 98%, at least 99%, or 100% sequence identity to SEQ ID No: 149; or
(14) the LC comprises the amino acid sequence having at least 80%, at least 85%, at least 90%, at least 95%, at least 98%, at least 99%, or 100% sequence identity to SEQ ID No: 164, and the HC comprises the amino acid sequence having at least 80%, at least 85%, at least 90%, at least 95%, at least 98%, at least 99%, or 100% sequence identity to SEQ ID No: 150; or
(15) the LC comprises the amino acid sequence having at least 80%, at least 85%, at least 90%, at least 95%, at least 98%, at least 99%, or 100% sequence identity to SEQ ID No: 165, and the HC comprises the amino acid sequence having at least 80%, at least 85%, at least 90%, at least 95%, at least 98%, at least 99%, or 100% sequence identity to SEQ ID No: 149; or
(16) the LC comprises the amino acid sequence having at least 80%, at least 85%, at least 90%, at least 95%, at least 98%, at least 99%, or 100% sequence identity to SEQ ID No: 166, and the HC comprises the amino acid sequence having at least 80%, at least 85%, at least 90%, at least 95%, at least 98%, at least 99%, or 100% sequence identity to SEQ ID No: 150.

In some preferred embodiments, the antibody or the antigen-binding fragment thereof of the invention has a binding activity to human CD200R1 and Cynomolgus macaques CD200R1. In some preferred embodiments, the antibody or the antigen-binding fragment thereof of the invention blocks the interaction between CD200R1 and CD200. In some preferred embodiments, the antibody or the antigen-binding fragment thereof of the invention inhibits CD200/CD200R1 signaling. In some preferred embodiments, the antibody or the antigen-binding fragment thereof of the invention does not activate CD200/CD200R1 signaling.

In some preferred embodiments, the antibody is a murine antibody, a chimeric antibody, a humanized antibody, or a human antibody.

In some preferred embodiments, the antibody is of an isotype selected from the group consisting of IgG, IgA, IgM, IgE and IgD. In some more preferred embodiments, the antibody is of a subtype selected from the group consisting of IgG1, IgG2, IgG3, and IgG4. In some preferred embodiments, the antigen binding fragment is selected from the group consisting of Fab, Fab′, F(ab′)2, Fd, Fd′, Fv, scFv, ds-scFv and dAb.

In some preferred embodiments, the antibody is a monoclonal antibody, a bi-specific or a multi-specific antibody. In some embodiments, the antibody is a bi-specific antibody further comprising a second antigen binding region specifically binding to a tumor associated antigen or an immune checkpoint molecule.

In some preferred embodiments, the antibody or antigen binding fragment is attached to a fluorescent label, radiolabel or cytotoxic agent.

In the third aspect, the invention provides a nucleic acid comprising a nucleotide sequence encoding the antibody or the antigen binding fragment thereof of the first or the second aspects of the invention.

In the fourth aspect, the invention provides a vector comprising the nucleic acid of the third aspect of the invention.

In the fifth aspect, the invention provides a host cell comprising the nucleic acid of the third aspect or the vector of the fourth aspect of the invention.

In the sixth aspect, the invention provides an antibody-drug conjugate (ADC), comprising the antibody or the antigen-binding fragment thereof of the first aspect of the invention or the bi-specific antibody of the second aspect of the invention.

In the seventh aspect, the invention provides a pharmaceutical composition comprising (i) the antibody or the antigen binding fragment thereof of the first or the second aspects of the invention, or the nucleic acid of the third aspect of the invention, or the vector of the forth aspect of the invention, or the host cell of the fifth aspect of the invention, or the ADC of the sixth aspect of the invention; and optionally (ii) a pharmaceutically acceptable carrier or excipient.

In some preferred embodiments of the seventh aspect, the composition further comprises a second therapeutic agent selected from the group consisting of an antibody, a chemotherapeutic agent, a siRNA, an antisense oligonucleotide, a polypeptide, and a small molecule drug.

In the eighth aspect, the invention provides a method of treating a cancer in a subject, comprising administering to the subject an effective amount of the antibody or the antigen binding fragment thereof, the nucleic acid, the vector, the host cell, the ADC, or the pharmaceutical composition of the invention. In some embodiments, the microenvironment of the cancer comprises T cells and/or myeloid cells on the surface of which CD200R1 is highly expressed. In some embodiments, the cancer is a cancer responsive to decreasing, inhibiting and/or blocking immune regulatory function or activity mediated by CD200R1.

In a ninth aspect, the invention provides an effective amount of the antibody or the antigen binding fragment thereof, the nucleic acid, the vector, the host cell, the ADC, or the pharmaceutical composition of the invention for use in a method of treating a cancer in a subject. In some embodiments, the microenvironment of the cancer comprises T cells and/or myeloid cells on the surface of which CD200R1 is highly expressed. In some embodiments, the cancer is a cancer responsive to decreasing, inhibiting and/or blocking immune regulatory function or activity mediated by CD200R1.

In a tenth aspect, the invention provides use of the antibody or the antigen binding fragment thereof, the nucleic acid, the vector, the host cell, the ADC, or the pharmaceutical composition of the invention in the manufacture of a medicament for treating a cancer in a subject. In some embodiments, the microenvironment of the cancer comprises T cells and/or myeloid cells on the surface of which CD200R1 is highly expressed. In some embodiments, the cancer is a cancer responsive to decreasing, inhibiting and/or blocking immune regulatory function or activity mediated by CD200R1.

In some preferred embodiments of the eighth, ninth, or tenth aspects, the cancer is selected from the group consisting of renal cancer, pancreatic ductal adenocarcinoma, prostate cancer, melanoma, liver cancer, breast cancer, lung cancer (e.g., lung squamous cell carcinoma), mesothelioma, squamous cell carcinoma, ovarian cancer, papillary thyroid carcinoma, uterine carcinoma, brain cancer, esophageal carcinoma, stomach carcinoma, colon cancer, rectal cancer, head and neck carcinoma, liposarcoma, leukemia, and myeloma.

In some preferred embodiments of the eighth, ninth, or tenth aspects, the method further comprises administering to the subject a second therapeutic agent. In some more preferred embodiments of the eighth, ninth, or tenth aspects, the second therapeutic agent is selected from an antibody, a chemotherapeutic agent, a siRNA, an antisense oligonucleotide, a polypeptide and a small molecule drug.

In an eleventh aspect, the invention provides a method for determining a subject suffering from a cancer or having a risk of developing a cancer, wherein the microenvironment of the cancer comprises T cells and/or myeloid cells on the surface of which CD200R1 is highly expressed, wherein the method comprises:
(a) obtaining a biological sample from the subject,
(b) contacting the sample with the antibody or the antigen binding fragment thereof of the first aspect of the invention, and
(c) detecting binding of the antibody to the sample,
wherein an increase in binding of the antibody or antigen binding fragment thereof to the sample as compared to binding of the antibody or antigen binging fragment thereof to a control sample indicates that the subject is suffering from a cancer or has a risk of developing a cancer.

In some embodiments, the cancer is a cancer responsive to decreasing, inhibiting and/or blocking immune regulatory function or activity mediated by CD200R1.

In a twelfth aspect, the invention provides a method for imaging a cancer in a subject, wherein the microenvironment of the cancer comprises T cells and/or myeloid cells on the surface of which CD200R1 is highly expressed, wherein the method comprises:
(a) administering the antibody or antigen binding fragment thereof of the first aspect of the invention, wherein the antibody is conjugated to a detectable marker, and
(b) detecting the presence of the marker.

In some embodiments, the cancer is a cancer responsive to decreasing, inhibiting and/or blocking immune regulatory function or activity mediated by CD200R1.

### BRIEF DESCRIPTION OF THE DRAWINGS

FIG. 1A and FIG. 1B illustrates the expression of human CD200R1 on human PBMCs from healthy donor and tumor infiltration lymphocytes from renal cell carcinoma patient.

FIG. 2 illustrates the expression profile of mouse CD200R1 after PD1 treatment in a mouse HKP tumor model.

FIGS. 3A˜3C illustrates the binding of anti-CD200R1 antibodies to CHOK1 cells overexpressing human CD200R1 isoform a (CHOK1/hCD200R1a).

FIGS. 4A˜4C illustrates the binding of anti-CD200R1 antibodies to CHOK1 cells overexpressing cyno CD200R1a (CHOK1/cynoCD200R1a).

FIG. 5 illustrates the efficacy of anti-CD200R1 antibodies to block the binding of CD200 protein to CHOK1/hCD200R1a.

FIGS. 6A and 6B illustrates the efficacy of anti-CD200R1 antibodies to block CD200-induced PathHunter CD200R1a signaling.

FIGS. 7A and 7B illustrates the binding of variants of anti-CD200R1 antibodies to CHOK1/hCD200R1a.

FIGS. 8A and 8B illustrates the binding of variants of anti-CD200R1 antibodies to CHOK1/cynoCD200R1a.

FIGS. 9A and 9B illustrates the efficacy of variants of anti-CD200R1 antibodies to block the binding of CD200 protein to CHOK1/hCD200R1a.

FIGS. 10A-101D illustrates the efficacy of variants of anti-CD200R1 antibodies to block CD200-induced PathHunter CD200R1a signaling.

FIGS. 11A and 11B illustrates the abilities of variants of anti-CD200R1 antibodies to induce PathHunter CD200R1a signaling.

FIG. 12 illustrates the binding of anti-CD200R1 antibodies to recombinant human CD200R1L protein.

FIGS. 13A and 13B illustrates the binding of anti-CD200R1 antibodies to CHOK1 expressing different CD200R1 isoforms, CHOK1/hCD200R1a and CHOK1/hCD200R1d.

FIGS. 14A and 14B illustrates the efficacy of anti-CD200R1 antibodies to block the binding of CD200 protein to CHOK1/hCD200R1a and CHOK1/hCD200R1d.

FIGS. 15A and 15B illustrates the binding of human anti-CD200R1 antibodies on human PBMC from healthy donor and on tumor infiltration lymphocytes from renal cell carcinoma patient.

FIGS. 16A and 16B illustrates the function of anti-CD200R1 antibody to induce IL12 production in antigen recall assay (FIG. 16A) and to enhance anti-PD1 induced IFNγ production in MLR assay (FIG. 16B).

FIG. 17 illustrates the anti-tumor effects of anti-CD200R1 antibodies in NCI-H226 CDX model in humanized NSG mice.

FIG. 18 illustrates the anti-tumor effects of anti-CD200R1 antibody in OVCAR3 CDX model in humanized NSG mice.

FIG. 19 illustrates the anti-tumor effects of anti-CD200R1 antibodies in NCI-H292 CDX model in humanized NSG mice.

FIG. 20 illustrates the anti-tumor effects of anti-CD200R1 antibodies in MDA-MB-231 CDX model in humanized NSG mice.

### SEQUENCE LISTING

The sequences of the light chain, heavy chain, the variable region of light chain, the variable region of heavy chain, the CDRs of the light chain and heavy chain are indicated in Tables 1-3 below. The CDR sequences are defined according to Chothia numbering system.

TABLE 1
Sequences of the heavy chain and light chain
of the antibodies of the invention

| | | SEQ | | SEQ |
|---|---|---|---|---|
| Clone | Heavy chain | ID No. | Light chain | ID No. |
| PR005474 | QVQLVESGGGVVQPGRSLRLSCAA | 140 | DIVMTQSPLSLPVTPGEPAS | 157 |
| | SAFTFSSYGMHWVRQAPGKGLEWV | | ISCRSSQSLLHSNGYNYLDW | |
| | ALIWNDGSNKFYADSVKGRFTISR | | YLQKPGQSPQLLIYLGSNRA | |
| | DNSKNTLFLQMNSLRAEDTAVYYC | | SGVPDRFSGSGSGTDFTLKI | |
| | ARDRILGAGFDYWGQGTLVTVSSA | | SRVEAEDVGVYYCMQALQTP | |
| | STKGPSVFPLAPSSKSTSGGTAAL | | WTFGQGTKVEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGGP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALGAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR005509 | QVQLVESGGGVVQPGRSLRLSCAA | 141 | DIVMTQSPLSLPVTPGEPAS | 158 |
| | SGFTFSSYGFHWVRQAPGKGLEWL | | ISCRSSQSLLHSNGYNYLDW | |
| | GLIWSDGSNKYYADSVKGRFTISR | | YLQKPGQSPQLLIYLGSNRA | |
| | DNSKNTLYLQMNSLRAEDTAVYYC | | SGVPDRFSGSGSGTDFTLKI | |
| | ARDRIVGAGFDYWGQGTLVTVSSA | | SRVEAEDVGVYYCMQGLQTM | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGGP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALGAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR005512 | QVQLVESGGGVVQPGRSLRLSCVA | 142 | DIVMTQSPLSLPVTPGEPAS | 159 |
| | SGFTFSSYGMHWVRQAPGKGLEWV | | ISCRSSQSLLHSNGYNYLDW | |
| | AIIWNDGSNRYYADSVKGRFTISR | | YLQKPGQSPQLLIYLGSNRA | |
| | DNSKNTLYLQMNSLRAEDTAVYYC | | SGVPDRFSGSGSGTDFTLKI | |
| | ARDRIVGAGFDYWGQGTLVTVSSA | | SRLEAEDVGVYYCMQGLQTP | |
| | STKGPSVFPLAPSSKSTSGGTAAL | | WTFGQGTKVEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGGP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALGAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR005514 | QVQLVESGGGVVQPGRSLRLSCAA | 143 | DIVMTQSPLSLPVTPGEPAS | 158 |
| | SGFTFSSYGFHWVRQAPGKGLEWL | | ISCRSSQSLLHSNGYNYLDW | |
| | GLIWSDGSNKYYADSVKGRFTISR | | YLQKPGQSPQLLIYLGSNRA | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCMQGLQTM | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGGP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALGAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR006475 | QVQLQESGPGLVKPSGTLSLTCAV | 145 | EIVLTQSPATLSVSPGERAT | 161 |
| | SGASISSGHWWSWVRQPPGKGLEW | | LSCRASQSVSSNLAWYQQKP | |
| | IGEIYHSGSTNYTPSLKSRVTISV | | GQAPRLLIYGASTRATGIPA | |
| | DKSKNQFSLKLTSVTAADTAVYYC | | RFSGSGSGTEFTLTISSLQS | |
| | AREASYHYGSGSYYTPFDFWGQGT | | EDFALYYCQQYNNWIIFGQG | |
| | LVTVSSASTKGPSVFPLAPSSKST | | TRLEIKRTVAAPSVFIFPPS | |
| | SGGTAALGCLVKDYFPEPVTVSWN | | DEQLKSGTASVVCLLNNFYP | |
| | SGALTSGVHTFPAVLQSSGLYSLS | | REAKVQWKVDNALQSGNSQE | |
| | SVVTVPSSSLGTQTYICNVNHKPS | | SVTEQDSKDSTYSLSSTLTL | |
| | NTKVDKKVEPKSCDKTHTCPPCPA | | SKADYEKHKVYACEVTHQGL | |
| | PEAAGGPSVFLFPPKPKDTLMISR | | SSPVTKSFNRGEC | |
| | TPEVTCVVVDVSHEDPEVKFNWYV | | | |
| | DGVEVHNAKTKPREEQYNSTYRVV | | | |
| | SVLTVLHQDWLNGKEYKCKVSNKA | | | |
| | LGAPIEKTISKAKGQPREPQVYTL | | | |
| | PPSREEMTKNQVSLTCLVKGFYPS | | | |
| | DIAVEWESNGQPENNYKTTPPVLD | | | |
| | SDGSFFLYSKLTVDKSRWQQGNVF | | | |
| | SCSVMHEALHNHYTQKSLSLSPGK | | | |
| PR006480 | QVQLVQSGGGVVQPGRSLRLSCAA | 146 | DIQMTQSPPSLSASVGDRVT | 162 |
| | SGFTFSSYGMHWVRQAPGKGLEWV | | ITCRASQSISSYLNWFQQKA | |
| | ALIWYDGSNKYYADSVKGRFTISR | | GKAPKLLISATSNLQSGVPS | |
| | DNSKNTLYLQMNSLRAEDTAVYYC | | RFSGSGSGTDFTLTISSLQP | |
| | ARSVLLFDSWGQGTLVTVSSASTK | | EDLATYYCQQSYSSPYTFGQ | |
| | GPSVFPLAPSSKSTSGGTAALGCL | | GTKVEIKRTVAAPSVFIFPP | |
| | VKDYFPEPVTVSWNSGALTSGVHT | | SDEQLKSGTASVVCLLNNFY | |
| | FPAVLQSSGLYSLSSVVTVPSSSL | | PREAKVQWKVDNALQSGNSQ | |
| | GTQTYICNVNHKPSNTKVDKKVEP | | ESVTEQDSKDSTYSLSSTLT | |
| | KSCDKTHTCPPCPAPEAAGGPSVF | | LSKADYEKHKVYACEVTHQG | |
| | LFPPKPKDTLMISRTPEVTCVVVD | | LSSPVTKSFNRGEC | |
| | VSHEDPEVKFNWYVDGVEVHNAKT | | | |
| | KPREEQYNSTYRVVSVLTVLHQDW | | | |
| | LNGKEYKCKVSNKALGAPIEKTIS | | | |
| | KAKGQPREPQVYTLPPSREEMTKN | | | |
| | QVSLTCLVKGFYPSDIAVEWESNG | | | |
| | QPENNYKTTPPVLDSDGSFFLYSK | | | |
| | LTVDKSRWQQGNVFSCSVMHEALH | | | |
| | NHYTQKSLSLSPGK | | | |
| PR006839 | QVQLVESGGGVVQPGRSLRLSCAA | 147 | DIVMTQSPLSLPVTPGEPAS | 158 |
| | SGFTFSSYGFHWVRQAPGKGLEWL | | ISCRSSQSLLHSNGYNYLDW | |
| | GLIWSDGSNKYYADSVKGRFTISR | | YLQKPGQSPQLLIYLGSNRA | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCMQGLQTM | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGAP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR006895 | QVQLVQSGGGVVQPGRSLRLSCAA | 148 | DIQMTQSPPSLSASVGDRVT | 162 |
| | SGFTFSSYGMHWVRQAPGKGLEWV | | ITCRASQSISSYLNWFQQKA | |
| | ALIWYDGSNKYYADSVKGRFTISR | | GKAPKLLISATSNLQSGVPS | |
| | DNSKNTLYLQMNSLRAEDTAVYYC | | RFSGSGSGTDFTLTISSLQP | |
| | ARSVLLFDSWGQGTLVTVSSASTK | | EDLATYYCQQSYSSPYTFGQ | |
| | GPSVFPLAPSSKSTSGGTAALGCL | | GTKVEIKRTVAAPSVFIFPP | |
| | VKDYFPEPVTVSWNSGALTSGVHT | | SDEQLKSGTASVVCLLNNFY | |
| | FPAVLQSSGLYSLSSVVTVPSSSL | | PREAKVQWKVDNALQSGNSQ | |
| | GTQTYICNVNHKPSNTKVDKKVEP | | ESVTEQDSKDSTYSLSSTLT | |
| | KSCDKTHTCPPCPAPEAAGAPSVF | | LSKADYEKHKVYACEVTHQG | |
| | LFPPKPKDTLMISRTPEVTCVVVD | | LSSPVTKSFNRGEC | |
| | VSHEDPEVKFNWYVDGVEVHNAKT | | | |
| | KPREEQYNSTYRVVSVLTVLHQDW | | | |
| | LNGKEYKCKVSNKALPAPIEKTIS | | | |
| | KAKGQPREPQVYTLPPSREEMTKN | | | |
| | QVSLTCLVKGFYPSDIAVEWESNG | | | |
| | QPENNYKTTPPVLDSDGSFFLYSK | | | |
| | LTVDKSRWQQGNVFSCSVMHEALH | | | |
| | NHYTQKSLSLSPGK | | | |
| PR007221 | QVQLVQSGGGVVQPGRSLRLSCAA | 149 | DIQLTQSPPSLSASVGDRVT | 163 |
| | SGFTFSSYGMHWVRQAPGKGLEWV | | ITCRASQSISSYLNWFQQKA | |
| | ALIWYDASYKYYADSVKGRFTISR | | GKAPKLLISATSNLQSGVPS | |
| | DNSKNTLYLQMNSLRAEDTAVYYC | | RFSGSGSGTDFTLTISSLQP | |
| | ARSVLLFDSWGQGTLVTVSSASTK | | EDLATYYCQQTLSSPYTFGQ | |
| | GPSVFPLAPSSKSTSGGTAALGCL | | GTKVEIKRTVAAPSVFIFPP | |
| | VKDYFPEPVTVSWNSGALTSGVHT | | SDEQLKSGTASVVCLLNNFY | |
| | FPAVLQSSGLYSLSSVVTVPSSSL | | PREAKVQWKVDNALQSGNSQ | |
| | GTQTYICNVNHKPSNTKVDKKVEP | | ESVTEQDSKDSTYSLSSTLT | |
| | KSCDKTHTCPPCPAPEAAGAPSVF | | LSKADYEKHKVYACEVTHQG | |
| | LFPPKPKDTLMISRTPEVTCVVVD | | LSSPVTKSFNRGEC | |
| | VSHEDPEVKFNWYVDGVEVHNAKT | | | |
| | KPREEQYNSTYRVVSVLTVLHQDW | | | |
| | LNGKEYKCKVSNKALPAPIEKTIS | | | |
| | KAKGQPREPQVYTLPPSREEMTKN | | | |
| | QVSLTCLVKGFYPSDIAVEWESNG | | | |
| | QPENNYKTTPPVLDSDGSFFLYSK | | | |
| | LTVDKSRWQQGNVFSCSVMHEALH | | | |
| | NHYTQKSLSLSPGK | | | |
| PR007223 | QVQLVQSGGGVVQPGRSLRLSCAA | 150 | DIQLTQSPPSLSASVGDRVT | 164 |
| | SGFTFSSYGMHWVRQAPGKGLEWV | | ITCRASQSISSYLNWFQQKA | |
| | ALIWYDASNKYYADSVKGRFTISR | | GKAPKLLISATSNLQSGVPS | |
| | DNSKNTLYLQMNSLRAEDTAVYYC | | RFSGSGSGTDFTLTISSLQP | |
| | ARSVLLFNMWGQGTLVTVSSASTK | | EDLATYYCQQTYSSPYTFGQ | |
| | GPSVFPLAPSSKSTSGGTAALGCL | | GTKVEIKRTVAAPSVFIFPP | |
| | VKDYFPEPVTVSWNSGALTSGVHT | | SDEQLKSGTASVVCLLNNFY | |
| | FPAVLQSSGLYSLSSVVTVPSSSL | | PREAKVQWKVDNALQSGNSQ | |
| | GTQTYICNVNHKPSNTKVDKKVEP | | ESVTEQDSKDSTYSLSSTLT | |
| | KSCDKTHTCPPCPAPEAAGAPSVF | | LSKADYEKHKVYACEVTHQG | |
| | LFPPKPKDTLMISRTPEVTCVVVD | | LSSPVTKSFNRGEC | |
| | VSHEDPEVKFNWYVDGVEVHNAKT | | | |
| | KPREEQYNSTYRVVSVLTVLHQDW | | | |
| | LNGKEYKCKVSNKALPAPIEKTIS | | | |
| | KAKGQPREPQVYTLPPSREEMTKN | | | |
| | QVSLTCLVKGFYPSDIAVEWESNG | | | |
| | QPENNYKTTPPVLDSDGSFFLYSK | | | |
| | LTVDKSRWQQGNVFSCSVMHEALH | | | |
| | NHYTQKSLSLSPGK | | | |
| PR007442 | QVQLVQSGGGVVQPGRSLRLSCAA | 149 | DIQMTQSPPSLSASVGDRVT | 165 |
| | SGFTFSSYGMHWVRQAPGKGLEWV | | ITCRASQSISSYLNWFQQKA | |
| | ALIWYDASYKYYADSVKGRFTISR | | GKAPKLLISATSNLQSGVPS | |
| | DNSKNTLYLQMNSLRAEDTAVYYC | | RFSGSGSGTDFTLTISSLQP | |
| | ARSVLLFDSWGQGTLVTVSSASTK | | EDLATYYCQQTLSSPYTFGQ | |
| | GPSVFPLAPSSKSTSGGTAALGCL | | GTKVEIKRTVAAPSVFIFPP | |
| | VKDYFPEPVTVSWNSGALTSGVHT | | SDEQLKSGTASVVCLLNNFY | |
| | FPAVLQSSGLYSLSSVVTVPSSSL | | PREAKVQWKVDNALQSGNSQ | |
| | GTQTYICNVNHKPSNTKVDKKVEP | | ESVTEQDSKDSTYSLSSTLT | |
| | KSCDKTHTCPPCPAPEAAGAPSVF | | LSKADYEKHKVYACEVTHQG | |
| | LFPPKPKDTLMISRTPEVTCVVVD | | LSSPVTKSFNRGEC | |
| | VSHEDPEVKFNWYVDGVEVHNAKT | | | |
| | KPREEQYNSTYRVVSVLTVLHQDW | | | |
| | LNGKEYKCKVSNKALPAPIEKTIS | | | |
| | KAKGQPREPQVYTLPPSREEMTKN | | | |
| | QVSLTCLVKGFYPSDIAVEWESNG | | | |
| | QPENNYKTTPPVLDSDGSFFLYSK | | | |
| | LTVDKSRWQQGNVFSCSVMHEALH | | | |
| | NHYTQKSLSLSPGK | | | |
| PR007444 | QVQLVQSGGGVVQPGRSLRLSCAA | 150 | DIQMTQSPPSLSASVGDRVT | 166 |
| | SGFTFSSYGMHWVRQAPGKGLEWV | | ITCRASQSISSYLNWFQQKA | |
| | ALIWYDASNKYYADSVKGRFTISR | | GKAPKLLISATSNLQSGVPS | |
| | DNSKNTLYLQMNSLRAEDTAVYYC | | RFSGSGSGTDFTLTISSLQP | |
| | ARSVLLFNMWGQGTLVTVSSASTK | | EDLATYYCQQTYSSPYTFGQ | |
| | GPSVFPLAPSSKSTSGGTAALGCL | | GTKVEIKRTVAAPSVFIFPP | |
| | VKDYFPEPVTVSWNSGALTSGVHT | | SDEQLKSGTASVVCLLNNFY | |
| | FPAVLQSSGLYSLSSVVTVPSSSL | | PREAKVQWKVDNALQSGNSQ | |
| | GTQTYICNVNHKPSNTKVDKKVEP | | ESVTEQDSKDSTYSLSSTLT | |
| | KSCDKTHTCPPCPAPEAAGAPSVF | | LSKADYEKHKVYACEVTHQG | |
| | LFPPKPKDTLMISRTPEVTCVVVD | | LSSPVTKSFNRGEC | |
| | VSHEDPEVKFNWYVDGVEVHNAKT | | | |
| | KPREEQYNSTYRVVSVLTVLHQDW | | | |
| | LNGKEYKCKVSNKALPAPIEKTIS | | | |
| | KAKGQPREPQVYTLPPSREEMTKN | | | |
| | QVSLTCLVKGFYPSDIAVEWESNG | | | |
| | QPENNYKTTPPVLDSDGSFFLYSK | | | |
| | LTVDKSRWQQGNVFSCSVMHEALH | | | |
| | NHYTQKSLSLSPGK | | | |
| PR007630 | QVQLVESGGGVVQPGRSLRLSCAA | 151 | DIVMTQSPLSLPVTPGEPAS | 167 |
| | SGFTFSSYGFHWVRQAPGKGLEWL | | ISCRSSQSLLHRSGYNYLDW | |
| | GIIWSDASNKYYADSVKGRFTISR | | YLQKPGQSPQLLIYLGSYRA | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCMQGLQTM | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGAP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR007631 | QVQLVESGGGVVQPGRSLRLSCAA | 152 | DIVMTQSPLSLPVTPGEPAS | 168 |
| | SGFTFSSYGFHWVRQAPGKGLEWL | | ISCRSSQSLLHRSGYNYLDW | |
| | GLIWSDARHKDYADSVKGRFTISR | | YLQKPGQSPQLLIYLGSYRW | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCMQGLQTM | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGAP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR007972 | QVQLVESGGGVVQPGRSLRLSCAA | 153 | DIVMTQSPLSLPVTPGEPAS | 169 |
| | SGFTFSYYGFHWVRQAPGKGLEWL | | ISCRSSQSLLHRNGYNYLDW | |
| | GIIWSDASNKYYADSVKGRFTISR | | YLQKPGQSPQLLIYLGSNRA | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCQQGIQTE | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGAP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR007973 | QVQLVESGGGVVQPGRSLRLSCAA | 151 | DIVMTQSPLSLPVTPGEPAS | 170 |
| | SGFTFSSYGFHWVRQAPGKGLEWL | | ISCRSSQSLLHRNGYNYLDW | |
| | GIIWSDASNKYYADSVKGRFTISR | | YLQKPGQSPQLLIYLGSYRA | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCQQGIQTE | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGAP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR007974 | QVQLVESGGGVVQPGRSLRLSCAA | 154 | DIVMTQSPLSLPVTPGEPAS | 171 |
| | SGFTFSYYGFHWVRQAPGKGLEWL | | ISCRSSQSLLHRSGYNYLDW | |
| | GLIWSDARHKDYADSVKGRFTISR | | YLQKPGQSPQLLIYLGSYRA | |
| | DNSKNTLYLQMNSLRAEDTAVYYC | | SGVPDRFSGSGSGTDFTLKI | |
| | ARDRIVGEGFDYWGQGTLVTVSSA | | SRVEAEDVGVYYCQQGIQTT | |
| | STKGPSVFPLAPSSKSTSGGTAAL | | YTFGQGTKLEIKRTVAAPSV | |
| | GCLVKDYFPEPVTVSWNSGALTSG | | FIFPPSDEQLKSGTASVVCL | |
| | VHTFPAVLQSSGLYSLSSVVTVPS | | LNNFYPREAKVQWKVDNALQ | |
| | SSLGTQTYICNVNHKPSNTKVDKK | | SGNSQESVTEQDSKDSTYSL | |
| | VEPKSCDKTHTCPPCPAPEAAGAP | | SSTLTLSKADYEKHKVYACE | |
| | SVFLFPPKPKDTLMISRTPEVTCV | | VTHQGLSSPVTKSFNRGEC | |
| | VVDVSHEDPEVKFNWYVDGVEVHN | | | |
| | AKTKPREEQYNSTYRVVSVLTVLH | | | |
| | QDWLNGKEYKCKVSNKALPAPIEK | | | |
| | TISKAKGQPREPQVYTLPPSREEM | | | |
| | TKNQVSLTCLVKGFYPSDIAVEWE | | | |
| | SNGQPENNYKTTPPVLDSDGSFFL | | | |
| | YSKLTVDKSRWQQGNVFSCSVMHE | | | |
| | ALHNHYTQKSLSLSPGK | | | |
| PR002903 | QVQLQQSGSELKKPGASVKISCKA | 138 | DIQMTQSPSSLSASIGDRVT | 155 |
| | SGYSFTDYIILWVRQNPGKGLEWI | | ITCKASQDINSYLSWFQQKP | |
| | GHIDPYYGSSNYNLKFKGRVTITA | | GKAPKLLIYRANRLVDGVPS | |
| | DQSTTTAYMELSSLRSEDTAVYYC | | RFSGSGSGTDYTLTISSLQP | |
| | GRSKRDYFDYWGQGTTLTVSSAST | | EDFAVYYCLQYDEFPYTFGG | |
| | KGPSVFPLAPCSRSTSESTAALGC | | GTKLEIKRTVAAPSVFIFPP | |
| | LVKDYFPEPVTVSWNSGALTSGVH | | SDEQLKSGTASVVCLLNNFY | |
| | TFPAVLQSSGLYSLSSVVTVPSSN | | PREAKVQWKVDNALQSGNSQ | |
| | FGTQTYTCNVDHKPSNTKVDKTVE | | ESVTEQDSKDSTYSLSSTLT | |
| | RKCCVECPPCPAPPVAGPSVFLFP | | LSKADYEKHKVYACEVTHQG | |
| | PKPKDTLMISRTPEVTCVVVDVSQ | | LSSPVTKSFNRGEC | |
| | EDPEVQFNWYVDGVEVHNAKTKPR | | | |
| | EEQFNSTYRVVSVLTVLHQDWLNG | | | |
| | KEYKCKVSNKGLPSSIEKTISKAK | | | |
| | GQPREPQVYTLPPSQEEMTKNQVS | | | |
| | LTCLVKGFYPSDIAVEWESNGQPE | | | |
| | NNYKTTPPVLDSDGSFFLYSRLTV | | | |
| | DKSRWQEGNVFSCSVMHEALHNHY | | | |
| | TQKSLSLSLG | | | |
| PR002906 | QVQLQESGPGLVKPSETLSLTCTV | 139 | DIQMTQSPSSLSASVGDRVT | 156 |
| | SGYTITSGYDWSWIRQPPGKGLEW | | ITCKASKNIRSYLAWYQQKP | |
| | IGYINYGGSTNYKPSLGSRVTISV | | GKAPKLLIYSGSTLHSGVPS | |
| | DTSKNQFSLKLSSVTAADTAVYYC | | RFSGSGSGTDFTLTISSLQP | |
| | ARYNEYKSYIYDWYFDFWGQGTLV | | EDFATYYCQQHHEYPLTFGQ | |
| | TVSSASTKGPSVFPLAPSSKSTSG | | GTKVEIKRTVAAPSVFIFPP | |
| | GTAALGCLVKDYFPEPVTVSWNSG | | SDEQLKSGTASVVCLLNNFY | |
| | ALTSGVHTFPAVLQSSGLYSLSSV | | PREAKVQWKVDNALQSGNSQ | |
| | VTVPSSSLGTQTYICNVNHKPSNT | | ESVTEQDSKDSTYSLSSTLT | |
| | KVDKKVEPKSCDKTHTCPPCPAPE | | LSKADYEKHKVYACEVTHQG | |
| | LLGGPSVFLFPPKPKDTLMISRTP | | LSSPVTKSFNRGEC | |
| | EVTCVVVDVSHEDPEVKFNWYVDG | | | |
| | VEVHNAKTKPREEQYNSTYRVVSV | | | |
| | LTVLHQDWLNGKEYKCKVSNKALP | | | |
| | APIEKTISKAKGQPREPQVYTLPP | | | |
| | SREEMTKNQVSLTCLVKGFYPSDI | | | |
| | AVEWESNGQPENNYKTTPPVLDSD | | | |
| | GSFFLYSKLTVDKSRWQQGNVFSC | | | |
| | SVMHEALHNHYTQKSLSLSPGK | | | |
| PR006207 | QVQLVQSGAEVKKPGASVKVSCKA | 144 | EIVLTQSPDFQSVTPKEKVT | 160 |
| | SGFSFSSGYYMAWVRQAPGQGLEW | | ITCQASESIDSYLLWYQQKP | |
| | MGLIGVGSGSLWYAQKFQGRVTMT | | DQSPKLLIKQASTLASGVPS | |
| | RDTSTSTVYMELSSLRSEDTAVYY | | RFSGSGSGTDFTLTINSLEA | |
| | CARHFALSDPFNLWGQGTLVTVSS | | EDAATYYCQNYYDISSNDFG | |
| | ASTKGPSVFPLAPSSKSTSGGTAA | | GGTKVEIKRTVAAPSVFIFP | |
| | LGCLVKDYFPEPVTVSWNSGALTS | | PSDEQLKSGTASVVCLLNNF | |
| | GVHTFPAVLQSSGLYSLSSVVTVP | | YPREAKVQWKVDNALQSGNS | |
| | SSSLGTQTYICNVNHKPSNTKVDK | | QESVTEQDSKDSTYSLSSTL | |
| | KVEPKSCDKTHTCPPCPAPEAAGG | | TLSKADYEKHKVYACEVTHQ | |
| | PSVFLFPPKPKDTLMISRTPEVTC | | GLSSPVTKSFNRGEC | |
| | VVVDVSHEDPEVKFNWYVDGVEVH | | | |
| | NAKTKPREEQYNSTYRVVSVLTVL | | | |
| | HQDWLNGKEYKCKVSNKALGAPIE | | | |
| | KTISKAKGQPREPQVYTLPPSREE | | | |
| | MTKNQVSLTCLVKGFYPSDIAVEW | | | |
| | ESNGQPENNYKTTPPVLDSDGSFF | | | |
| | LYSKLTVDKSRWQQGNVFSCSVMH | | | |
| | EALHNHYTQKSLSLSPGK | | | |

TABLE 2
Sequences of the heavy chain variable region and light
chain variable region of the antibodies of the invention

| | Heavy chain | SEQ | Light chain | SEQ |
|---|---|---|---|---|
| Clone | variable region | ID No. | variable region | ID No. |
| PR005474 | QVQLVESGGGVVQPGRSLRLSCA | 108 | DIVMTQSPLSLPVTPGEPASI | 123 |
| | ASAFTFSSYGMHWVRQAPGKGLE | | CRSSQSLLHSNGYNYLDWYLS | |
| | WVALIWNDGSNKFYADSVKGRFT | | QKPGQSPQLLIYLGSNRASGV | |
| | ISRDNSKNTLFLQMNSLRAEDTA | | PDRFSGSGSGTDFTLKISRVE | |
| | VYYCARDRILGAGFDYWGQGTLV | | AEDVGVYYCMQALQTPWTFGQ | |
| | TVSS | | GTKVEIK | |
| PR005509 | QVQLVESGGGVVQPGRSLRLSCA | 109 | DIVMTQSPLSLPVTPGEPASI | 124 |
| | ASGFTFSSYGFHWVRQAPGKGLE | | SCRSSQSLLHSNGYNYLDWYL | |
| | WLGLIWSDGSNKYYADSVKGRFT | | QKPGQSPQLLIYLGSNRASGV | |
| | ISRDNSKNTLYLQMNSLRAEDTA | | PDRFSGSGSGTDFTLKISRVE | |
| | VYYCARDRIVGAGFDYWGQGTLV | | AEDVGVYYCMQGLQTMYTFGQ | |
| | TVSS | | GTKLEIK | |
| PR005512 | QVQLVESGGGVVQPGRSLRLSCV | 110 | DIVMTQSPLSLPVTPGEPASI | 125 |
| | ASGFTFSSYGMHWVRQAPGKGLE | | SCRSSQSLLHSNGYNYLDWYL | |
| | WVAIIWNDGSNRYYADSVKGRFT | | QKPGQSPQLLIYLGSNRASGV | |
| | ISRDNSKNTLYLQMNSLRAEDTA | | PDRFSGSGSGTDFTLKISRLE | |
| | VYYCARDRIVGAGFDYWGQGTLV | | AEDVGVYYCMQGLQTPWTFGQ | |
| | TVSS | | GTKVEIK | |
| PR005514 | QVQLVESGGGVVQPGRSLRLSCA | 111 | DIVMTQSPLSLPVTPGEPASI | 124 |
| | ASGFTFSSYGFHWVRQAPGKGLE | | SCRSSQSLLHSNGYNYLDWYL | |
| | WLGLIWSDGSNKYYADSVKGRFT | | QKPGQSPQLLIYLGSNRASGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCMQGLQTMYTFGQ | |
| | TVSS | | GTKLEIK | |
| PR006475 | QVQLQESGPGLVKPSGTLSLTCA | 113 | EIVLTQSPATLSVSPGERATL | 127 |
| | VSGASISSGHWWSWVRQPPGKGL | | SCRASQSVSSNLAWYQQKPGQ | |
| | EWIGEIYHSGSTNYTPSLKSRVT | | APRLLIYGASTRATGIPARFS | |
| | ISVDKSKNQFSLKLTSVTAADTA | | GSGSGTEFTLTISSLQSEDFA | |
| | VYYCAREASYHYGSGSYYTPFDF | | LYYCQQYNNWIIFGQGTRLEI | |
| | WGQGTLVTVSS | | K | |
| PR006480 | QVQLVQSGGGVVQPGRSLRLSCA | 114 | DIQMTQSPPSLSASVGDRVTI | 128 |
| | ASGFTFSSYGMHWVRQAPGKGLE | | TCRASQSISSYLNWFQQKAGK | |
| | WVALIWYDGSNKYYADSVKGRFT | | APKLLISATSNLQSGVPSRFS | |
| | ISRDNSKNTLYLQMNSLRAEDTA | | GSGSGTDFTLTISSLQPEDLA | |
| | VYYCARSVLLFDSWGQGTLVTVS | | TYYCQQSYSSPYTFGQGTKVE | |
| | S | | IK | |
| PR006839 | QVQLVESGGGVVQPGRSLRLSCA | 111 | DIVMTQSPLSLPVTPGEPASI | 124 |
| | ASGFTFSSYGFHWVRQAPGKGLE | | SCRSSQSLLHSNGYNYLDWYL | |
| | WLGLIWSDGSNKYYADSVKGRFT | | QKPGQSPQLLIYLGSNRASGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCMQGLQTMYTFGQ | |
| | TVSS | | GTKLEIK | |
| PR006895 | QVQLVQSGGGVVQPGRSLRLSCA | 114 | DIQMTQSPPSLSASVGDRVTI | 128 |
| | ASGFTFSSYGMHWVRQAPGKGLE | | TCRASQSISSYLNWFQQKAGK | |
| | WVALIWYDGSNKYYADSVKGRFT | | APKLLISATSNLQSGVPSRFS | |
| | ISRDNSKNTLYLQMNSLRAEDTA | | GSGSGTDFTLTISSLQPEDLA | |
| | VYYCARSVLLFDSWGQGTLVTVS | | TYYCQQSYSSPYTFGQGTKVE | |
| | S | | IK | |
| PR007221 | QVQLVQSGGGVVQPGRSLRLSCA | 115 | DIQLTQSPPSLSASVGDRVTI | 129 |
| | ASGFTFSSYGMHWVRQAPGKGLE | | TCRASQSISSYLNWFQQKAGK | |
| | WVALIWYDASYKYYADSVKGRFT | | APKLLISATSNLQSGVPSRFS | |
| | ISRDNSKNTLYLQMNSLRAEDTA | | GSGSGTDFTLTISSLQPEDLA | |
| | VYYCARSVLLFDSWGQGTLVTVS | | TYYCQQTLSSPYTFGQGTKVE | |
| | S | | IK | |
| PR007223 | QVQLVQSGGGVVQPGRSLRLSCA | 116 | DIQLTQSPPSLSASVGDRVTI | 130 |
| | ASGFTFSSYGMHWVRQAPGKGLE | | TCRASQSISSYLNWFQQKAGK | |
| | WVALIWYDASNKYYADSVKGRFT | | APKLLISATSNLQSGVPSRFS | |
| | ISRDNSKNTLYLQMNSLRAEDTA | | GSGSGTDFTLTISSLQPEDLA | |
| | VYYCARSVLLFNMWGQGTLVTVS | | TYYCQQTYSSPYTFGQGTKVE | |
| | S | | IK | |
| PR007442 | QVQLVQSGGGVVQPGRSLRLSCA | 115 | DIQMTQSPPSLSASVGDRVTI | 131 |
| | ASGFTFSSYGMHWVRQAPGKGLE | | TCRASQSISSYLNWFQQKAGK | |
| | WVALIWYDASYKYYADSVKGRFT | | APKLLISATSNLQSGVPSRFS | |
| | ISRDNSKNTLYLQMNSLRAEDTA | | GSGSGTDFTLTISSLQPEDLA | |
| | VYYCARSVLLFDSWGQGTLVTVS | | TYYCQQTLSSPYTFGQGTKVE | |
| | S | | IK | |
| PR007444 | QVQLVQSGGGVVQPGRSLRLSCA | 116 | DIQMTQSPPSLSASVGDRVTI | 132 |
| | ASGFTFSSYGMHWVRQAPGKGLE | | TCRASQSISSYLNWFQQKAGK | |
| | WVALIWYDASNKYYADSVKGRFT | | APKLLISATSNLQSGVPSRFS | |
| | ISRDNSKNTLYLQMNSLRAEDTA | | GSGSGTDFTLTISSLQPEDLA | |
| | VYYCARSVLLFNMWGQGTLVTVS | | TYYCQQTYSSPYTFGQGTKVE | |
| | S | | IK | |
| PR007630 | QVQLVESGGGVVQPGRSLRLSCA | 117 | DIVMTQSPLSLPVTPGEPASI | 133 |
| | ASGFTFSSYGFHWVRQAPGKGLE | | SCRSSQSLLHRSGYNYLDWYL | |
| | WLGIIWSDASNKYYADSVKGRFT | | QKPGQSPQLLIYLGSYRASGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCMQGLQTMYTFGQ | |
| | TVSS | | GTKLEIK | |
| PR007631 | QVQLVESGGGVVQPGRSLRLSCA | 118 | DIVMTQSPLSLPVTPGEPASI | 134 |
| | ASGFTFSSYGFHWVRQAPGKGLE | | SCRSSQSLLHRSGYNYLDWYL | |
| | WLGLIWSDARHKDYADSVKGRFT | | QKPGQSPQLLIYLGSYRWSGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCMQGLQTMYTFGQ | |
| | TVSS | | GTKLEIK | |
| PR007972 | QVQLVESGGGVVQPGRSLRLSCA | 119 | DIVMTQSPLSLPVTPGEPASI | 135 |
| | ASGFTFSYYGFHWVRQAPGKGLE | | SCRSSQSLLHRNGYNYLDWYL | |
| | WLGIIWSDASNKYYADSVKGRFT | | QKPGQSPQLLIYLGSNRASGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCQQGIQTEYTFGQ | |
| | TVSS | | GTKLEIK | |
| PR007973 | QVQLVESGGGVVQPGRSLRLSCA | 117 | DIVMTQSPLSLPVTPGEPASI | 136 |
| | ASGFTFSSYGFHWVRQAPGKGLE | | SCRSSQSLLHRNGYNYLDWYL | |
| | WLGIIWSDASNKYYADSVKGRFT | | QKPGQSPQLLIYLGSYRASGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCQQGIQTEYTFGQ | |
| | TVSS | | GTKLEIK | |
| PR007974 | QVQLVESGGGVVQPGRSLRLSCA | 120 | DIVMTQSPLSLPVTPGEPASI | 137 |
| | ASGFTFSYYGFHWVRQAPGKGLE | | SCRSSQSLLHRSGYNYLDWYL | |
| | WLGLIWSDARHKDYADSVKGRFT | | QKPGQSPQLLIYLGSYRASGV | |
| | ISRDNSKNTLYLQMNSLRAEDTA | | PDRFSGSGSGTDFTLKISRVE | |
| | VYYCARDRIVGEGFDYWGQGTLV | | AEDVGVYYCQQGIQTTYTFGQ | |
| | TVSS | | GTKLEIK | |
| PR002903 | QVQLQQSGSELKKPGASVKISCK | 106 | DIQMTQSPSSLSASIGDRVTI | 121 |
| | ASGYSFTDYIILWVRQNPGKGLE | | TCKASQDINSYLSWFQQKPGK | |
| | WIGHIDPYYGSSNYNLKFKGRVT | | APKLLIYRANRLVDGVPSRFS | |
| | ITADQSTTTAYMELSSLRSEDTA | | GSGSGTDYTLTISSLQPEDFA | |
| | VYYCGRSKRDYFDYWGQGTTLTV | | VYYCLQYDEFPYTFGGGTKLE | |
| | SS | | IK | |
| PR002906 | QVQLQESGPGLVKPSETLSLTCT | 107 | DIQMTQSPSSLSASVGDRVTI | 122 |
| | VSGYTITSGYDWSWIRQPPGKGL | | TCKASKNIRSYLAWYQQKPGK | |
| | EWIGYINYGGSTNYKPSLGSRVT | | APKLLIYSGSTLHSGVPSRFS | |
| | ISVDTSKNQFSLKLSSVTAADTA | | GSGSGTDFTLTISSLQPEDFA | |
| | VYYCARYNEYKSYIYDWYFDFWG | | TYYCQQHHEYPLTFGQGTKVE | |
| | QGTLVTVSS | | IK | |
| PR006207 | QVQLVQSGAEVKKPGASVKVSCK | 112 | EIVLTQSPDFQSVTPKEKVTI | 126 |
| | ASGFSFSSGYYMAWVRQAPGQGL | | TCQASESIDSYLLWYQQKPDQ | |
| | EWMGLIGVGSGSLWYAQKFQGRV | | SPKLLIKQASTLASGVPSRFS | |
| | TMTRDTSTSTVYMELSSLRSEDT | | GSGSGTDFTLTINSLEAEDAA | |
| | AVYYCARHFALSDPFNLWGQGTL | | TYYCQNYYDISSNDFGGGTKV | |
| | VTVSS | | EIK | |

TABLE 3
Sequences of the HCDR1-3 and LCDR1-3 of the
antibodies of the invention (Chothia system)

| | | SEQ | | SEQ | | SEQ | | SEQ | | SEQ | | SEQ |
|---|---|---|---|---|---|---|---|---|---|---|---|---|
| | | ID | | ID | | ID | | ID | | ID | | ID |
| Clone | HCDR1 | No. | HCDR2 | No. | HCDR3 | No. | LCDR1 | No. | LCDR2 | No. | LCDR3 | No. |
| PR005474 | AFTFSSY | 10 | WNDGS | 25 | DRILG | 44 | RSSQS | 62 | LGSNR | 76 | MQALQ | 91 |
| | | | N | | AGFDY | | LLHSN | | AS | | TPWT | |
| | | | | | | | GYNYL | | | | | |
| | | | | | | | D | | | | | |
| PR005509 | GFTFSSY | 11 | WSDGS | 26 | DRIVG | 45 | RSSQS | 62 | LGSNR | 76 | MQGLQ | 92 |
| | | | N | | AGFDY | | LLHSN | | AS | | TMYT | |
| | | | | | | | GYNYL | | | | | |
| | | | | | | | D | | | | | |
| PR005512 | GFTFSSY | 11 | WNDGS | 25 | DRIVG | 45 | RSSQS | 62 | LGSNR | 76 | MQGLQ | 93 |
| | | | N | | AGFDY | | LLHSN | | AS | | TPWT | |
| | | | | | | | GYNYL | | | | | |
| | | | | | | | D | | | | | |
| PR005514 | GFTFSSY | 11 | WSDGS | 26 | DRIVG | 46 | RSSQS | 62 | LGSNR | 76 | MQGLQ | 92 |
| | | | N | | EGFDY | | LLHSN | | AS | | TMYT | |
| | | | | | | | GYNYL | | | | | |
| | | | | | | | D | | | | | |
| PR006475 | GASISSG | 13 | YHSGS | 28 | EASYH | 48 | RASQS | 64 | GASTR | 78 | QQYNN | 95 |
| | H | | | | YGSGS | | VSSNL | | AT | | WII | |
| | | | | | YYTPF | | A | | | | | |
| | | | | | DF | | | | | | | |
| PR006480 | GFTFSSY | 11 | WYDGS | 29 | SVLLF | 49 | RASQS | 65 | ATSNL | 79 | QQSYS | 96 |
| | | | N | | DS | | ISSYL | | QS | | SPYT | |
| | | | | | | | N | | | | | |
| PR006839 | GFTFSSY | 11 | WSDGS | 26 | DRIVG | 46 | RSSQS | 62 | LGSNR | 76 | MQGLQ | 92 |
| | | | N | | EGFDY | | LLHSN | | AS | | TMYT | |
| | | | | | | | GYNYL | | | | | |
| | | | | | | | D | | | | | |
| PR006895 | GFTFSSY | 11 | WYDGS | 29 | SVLLF | 49 | RASQS | 65 | ATSNL | 79 | QQSYS | 96 |
| | | | N | | DS | | ISSYL | | QS | | SPYT | |
| | | | | | | | N | | | | | |
| PR007221 | GFTFSSY | 11 | WYDAS | 30 | SVLLF | 49 | RASQS | 65 | ATSNL | 79 | QQTLS | 97 |
| | | | Y | | DS | | ISSYL | | QS | | SPYT | |
| | | | | | | | N | | | | | |
| PR007223 | GFTFSSY | 11 | WYDAS | 31 | SVLLF | 50 | RASQS | 65 | ATSNL | 79 | QQTYS | 98 |
| | | | N | | NM | | ISSYL | | QS | | SPYT | |
| | | | | | | | N | | | | | |
| PR007442 | GFTFSSY | 11 | WYDAS | 30 | SVLLF | 49 | RASQS | 65 | ATSNL | 79 | QQTLS | 97 |
| | | | Y | | DS | | ISSYL | | QS | | SPYT | |
| | | | | | | | N | | | | | |
| PR007444 | GFTFSSY | 11 | WYDAS | 31 | SVLLF | 50 | RASQS | 65 | ATSNL | 79 | QQTYS | 98 |
| | | | N | | NM | | ISSYL | | QS | | SPYT | |
| | | | | | | | N | | | | | |
| PR007630 | GFTFSSY | 11 | WSDAS | 32 | DRIVG | 46 | RSSQS | 66 | LGSYR | 80 | MQGLQ | 92 |
| | | | N | | EGFDY | | LLHRS | | AS | | TMYT | |
| | | | | | | | GYNYL | | | | | |
| | | | | | | | D | | | | | |
| PR007631 | GFTFSSY | 11 | WSDAR | 33 | DRIVG | 46 | RSSQS | 66 | LGSYR | 81 | MQGLQ | 92 |
| | | | H | | EGFDY | | LLHRS | | WS | | TMYT | |
| | | | | | | | GYNYL | | | | | |
| | | | | | | | D | | | | | |
| PR007972 | GFTFSYY | 14 | WSDAS | 32 | DRIVG | 46 | RSSQS | 67 | LGSNR | 76 | QQGIQ | 99 |
| | | | N | | EGFDY | | LLHRN | | AS | | TEYT | |
| | | | | | | | GYNYL | | | | | |
| | | | | | | | D | | | | | |
| PR007973 | GFTFSSY | 11 | WSDAS | 32 | DRIVG | 46 | RSSQS | 67 | LGSYR | 80 | QQGIQ | 99 |
| | | | N | | EGFDY | | LLHRN | | AS | | TEYT | |
| | | | | | | | GYNYL | | | | | |
| | | | | | | | D | | | | | |
| PR007974 | GFTFSYY | 14 | WSDAR | 33 | DRIVG | 46 | RSSQS | 66 | LGSYR | 80 | QQGIQ | 100 |
| | | | H | | EGFDY | | LLHRS | | AS | | TTYT | |
| | | | | | | | GYNYL | | | | | |
| | | | | | | | D | | | | | |
| PR002903 | GYSFTDY | 8 | DPYYG | 23 | SKRDY | 42 | KASQD | 60 | RANRL | 74 | LQYDE | 89 |
| | | | S | | FDY | | INSYL | | VD | | FPYT | |
| | | | | | | | S | | | | | |
| PR002906 | GYTITSG | 9 | NYGGS | 24 | YNEYK | 43 | KASKN | 61 | SGSTL | 75 | QQHHE | 90 |
| | Y | | | | SYIYD | | IRSYL | | HS | | YPLT | |
| | | | | | WYFDF | | A | | | | | |
| PR006207 | GFSFSSG | 12 | GVGSG | 27 | HFALS | 47 | QASES | 63 | QASTL | 77 | QNYYD | 94 |
| | Y | | S | | DPFNL | | IDSYL | | AS | | ISSND | |
| | | | | | | | L | | | | | |

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings. The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland; Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

Unless indicated or defined otherwise, the term “comprise”, and variations thereof such as “comprises” and “comprising”, should be understood to imply the inclusion of a stated elements or step or group of elements or steps but not the exclusion of any other element or step or group of elements or steps. The term “comprising” encompasses “including” as well as “consisting” e.g., a composition “comprising” X may consist exclusively of X or may include something additional e.g., X+Y The term “about” in relation to a numerical value x is optional and means, for example, x±10% or x±5^{%}.

As used herein, the term “antibody” refers to an immunoglobulin molecule which has the ability to specifically bind to a specific antigen. An antibody often comprises a variable region and a constant region in each of a heavy chain and a light chain. The variable regions of the heavy and light chains of antibodies contain a binding domain that interacts with an antigen. The constant regions of antibodies may mediate the binding of the immunoglobulin to host tissues or factors, including various cells of the immune system (such as effector cells) and components of the complement system such as C1q, the first component in the classical pathway of complement activation. Accordingly, most antibodies have a heavy chain variable region (VH) and a light chain variable region (VL) that together form the portion of the antibody that binds to the antigen.

A “light chain variable region” (VL) or “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering system) in which corresponding residues between different heavy chains or between different light chains are assigned the same number. The present disclosure can use CDRs defined according to any of these numbering systems, although preferred embodiments use Chothia defined CDRs.

The term “antibody” as used herein should be understood in its broadest meaning, and includes monoclonal antibodies (including full-length monoclonal antibodies), polyclonal antibodies, antibody fragments, and multi-specific antibodies containing at least two different antigen binding regions (e.g., bispecific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

As used herein, the term “antigen binding fragment” of an antibody refers to one or more fragments of an antibody that retain the ability to specifically bind to an antigen (e.g., C200R1). It has been shown that the antigen binding function of an antibody can be performed by fragments of a full-length antibody.

Examples of antigen binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTALIMMUNOLOGY (Paul ed., 3.sup.rd ed. 1993); (iv) a Fd fragment consisting of the VH and CH1 domains; (v) a Fd′ fragment having VH and CH1 domains and one or more cysteine residues at the C-terminus of the CH1 domain; (vi) a Fv fragment consisting of the VL and VH domains of a single arm of an antibody, (vii) a dAb fragment (Ward et al., (1989) Nature 341:544-546), which consists of a VH domain; (viii) an isolated complementarity determining region (CDR); and (ix) a nanobody, a heavy chain variable region containing a single variable domain and two constant domains. Furthermore, although the two domains of the Fv fragment, V Land VH are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. Furthermore, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity.

These antigen binding fragments can be obtained using conventional techniques known to those with skill in the art, and the fragments are screened for utility in the same manner as are intact antibodies.

As used herein, the term “binding” or “specifically binding” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant (epitope) and an antigen-binding site on the antibody: the lesser the value of the KD, the stronger the binding strength between an antigenic determinant (epitope) and the antibody. Alternatively, the affinity can also be expressed as the affinity constant (KA), which is 1/KD.

Avidity is the measure of the strength of binding between an antibody and the pertinent antigen. Avidity is related to both the affinity between an antigenic determinant (epitope) and its antigen binding site on the antibody and the number of pertinent binding sites present on the antibody. Typically, an antibody will bind with a dissociation constant (KD) of 10⁻⁵ to 10⁻¹² M or less, and preferably 10⁻⁷ to 10⁻¹² M or less and more preferably 10⁻⁸ to 10⁻¹² M, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ M is generally considered to indicate non-specific binding. Specifically binding of an antibody to an antigen or antigenic determinant can be determined in any suitable manner known per se, including, for example, Scatchard analysis and/or competitive binding assays, such as radioimmunoassays (RIA), enzyme immunoassays (EIA), bio-layer interferometry (BLI) assay and sandwich competition assays, and the different variants thereof known per se in the art.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-2 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.

Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Nat. Acad Sci. USA 81: 140-144, 1984; Kyte & Doolittle, J Mol. Biol. 157: 105-132, 198 1, and Goldman et al., Ann. Rev. Biophys. Chem. 15: 321-353, 1986, all incorporated herein in their entirety by reference.

As used herein, the term “monoclonal antibody” refers to an antibody obtained from a substantially homogeneous antibody population. That is, the antibodies constituting the population are the same, except for possible naturally occurring mutations in small amount. Monoclonal antibodies are highly specific and are directed against a single antigen. The term “monoclonal antibody” herein is not limited to antibodies produced by hybridoma technology, and should not be interpreted as requiring production of antibodies by any specific method.

The term “bispecific antibody” is in the context of the present invention to be understood as an antibody having two different antigen-binding regions defined by different antibody sequences. This can be understood as different target binding but includes as well binding to different epitopes in one target.

As used herein, the term “tumor associated antigen” refers to an antigen that is differentially expressed in cancer cells compared to normal cells, and therefore can be used to target cancer cells.

As used herein, the term “bispecific T-cell engager” or “BiTE” refers to single polypeptide chain molecules that having two antigen-binding domains, one of which binds to a T-cell antigen and the second of which binds to an antigen present on the surface of a target (See, PCT Publication WO 05/061547; Baeuerle et al., 2008, Drugs of the Future 33: 137-147; Bargou, et al., 2008, Science 321:974-977, which are incorporated herein by reference in their entireties). Thus, the BiTE of the disclosure has an antigen binding region that binds to GPC3 and a second antigen binding region that is directed towards a T-cell antigen.

As used herein, the term “vector” is intended to refer to a nucleic acid molecule capable of transporting another nucleic acid to which it has been linked.

As used herein, the term “host cell” refers to a cell into which an expression vector has been introduced.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptom thereof. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g. cancers).

The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment, or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc.

The terms “hCD200R1” and “human CD200R1” are used interchangeably herein, and are refer to human CD200 receptor 1. The term includes any CD200R1 variants, isoforms and species homologs which are naturally expressed by human cells, including human T cells, or are expressed on cells transfected with genes or cDNA encoding the human CD200R1 which are naturally expressed on human cells. Preferably, the term refers to a wild-type human CD200R1 that has the amino acid sequence set forth in SEQ ID NO: 172.

The terms “cyno”, “cynomolgus”, and “Cynomolgus macaques” are used interchangeably herein, and are refer to cynomolgus monkey CD200 receptor 1. The term includes any CD200R1 variants, isoforms and species homologs which are naturally expressed by cynomolgus monkey cells, including cynomolgus monkey T cells, or are expressed on cells transfected with genes or cDNA encoding the cynomolgus monkey CD200R1 which are naturally expressed on cynomolgus monkey cells. Preferably, the term refers to a wild-type cynomolgus monkey CD200R1 that has the amino acid sequence set forth in SEQ ID NO: 175.

As used herein, “human CD200R1 antagonist antibody” or “anti-human CD200R antagonist antibody” refers to an antibody that binds to human CD200R1, and when administered in vitro or in vivo, results in the inhibition of CD200-CD200R1 signaling in the T cells or other types of cells in the microenvironment.

The terms “hCD200R1L” refer to human CD200 receptor 2. The term includes any CD200R1L variants, isoforms and species homologs which are naturally expressed by human cells, including human immune cells, or are expressed on cells transfected with genes or cDNA encoding the human CD200R1L which are naturally expressed on human cells. Preferably, the term refers to a wild-type human CD200R1L that has the amino acid sequence set forth in SEQ ID NO: 174.

### Anti-CD200R1 Antibodies

The invention provides an anti-CD200R1 antibody and an antigen-binding fragment thereof. In some embodiments, the anti-CD200R1 antibody specifically binds to human CD200R1 and Cynomolgus macaques CD200R1, but does not bind to mouse CD200R1. In some embodiments, the anti-CD200R1 antibody blocks the binding of CD200 and CD200R1, or blocks the interaction between CD200 and CD200R1. In some embodiments, the anti-CD200R1 antibody has stronger binding affinity to CD200R1 as compared to the binding of CD200 to CD200R1. In some embodiments, the anti-CD200R1 antibody binds to CD200R1 expressed on the surface of cells. In some embodiments, the anti-CD200R1 antibody blocks the binding of CD200 and CD200R1 expressed on the surface of cells. In some embodiments, the anti-CD200R1 antibody blocks the interaction between CD200 and CD200R1 expressed on the surface of cells. In some embodiments, the anti-CD200R1 antibody blocks the CD200-induced CD200R1 signaling in cells. In some embodiments, the anti-CD200R1 antibody do not bind to CD200R1L. In some embodiments, the anti-CD200R1 antibody has antagonistic activity but does not show agonistic activity.

In a first aspect, the invention provides an antibody that specifically binds to CD200R1, or an antigen binding fragment thereof, wherein the antibody comprises a light chain variable region (VL) and a heavy chain variable region (VH), and wherein
(1) the VH comprises the HCDRs 1-3 of a VH having the amino acid sequence set forth in SEQ ID NO: 117, and the VL comprises the LCDRs 1-3 of a VL having the amino acid sequence set forth in SEQ ID NO: 136; or
(2) the VH comprises the HCDRs 1-3 of a VH having the amino acid sequence set forth in SEQ ID NO: 111, and the VL comprises the LCDRs 1-3 of a VL having the amino acid sequence set forth in SEQ ID NO: 124; or
(3) the VH comprises the HCDRs 1-3 of a VH having the amino acid sequence set forth in SEQ ID NO: 117, and the VL comprises the LCDRs 1-3 of a VL having the amino acid sequence set forth in SEQ ID NO: 133; or
(4) the VH comprises the HCDRs 1-3 of a VH having the amino acid sequence set forth in SEQ ID NO: 118, and the VL comprises the LCDRs 1-3 of a VL having the amino acid sequence set forth in SEQ ID NO: 134; or
(5) the VH comprises the HCDRs 1-3 of a VH having the amino acid sequence set forth in SEQ ID NO: 119, and the VL comprises the LCDRs 1-3 of a VL having the amino acid sequence set forth in SEQ ID NO: 135; or
(6) the VH comprises the HCDRs 1-3 of a VH having the amino acid sequence set forth in SEQ ID NO: 120, and the VL comprises the LCDRs 1-3 of a VL having the amino acid sequence set forth in SEQ ID NO: 137; or
(7) the VH comprises the HCDRs 1-3 of a VH having the amino acid sequence set forth in SEQ ID NO: 108, and the VL comprises the LCDRs 1-3 of a VL having the amino acid sequence set forth in SEQ ID NO: 123; or
(8) the VH comprises the HCDRs 1-3 of a VH having the amino acid sequence set forth in SEQ ID NO: 109, and the VL comprises the LCDRs 1-3 of a VL having the amino acid sequence set forth in SEQ ID NO: 124; or
(9) the VH comprises the HCDRs 1-3 of a VH having the amino acid sequence set forth in SEQ ID NO: 110, and the VL comprises the LCDRs 1-3 of a VL having the amino acid sequence set forth in SEQ ID NO: 125; or
(10) the VH comprises the HCDRs 1-3 of a VH having the amino acid sequence set forth in SEQ ID NO: 114, and the VL comprises the LCDRs 1-3 of a VL having the amino acid sequence set forth in SEQ ID NO: 128; or
(11) the VH comprises the HCDRs 1-3 of a VH having the amino acid sequence set forth in SEQ ID NO: 115, and the VL comprises the LCDRs 1-3 of a VL having the amino acid sequence set forth in SEQ ID NO: 129; or
(12) the VH comprises the HCDRs 1-3 of a VH having the amino acid sequence set forth in SEQ ID NO: 116, and the VL comprises the LCDRs 1-3 of a VL having the amino acid sequence set forth in SEQ ID NO: 130; or
(13) the VH comprises the HCDRs 1-3 of a VH having the amino acid sequence set forth in SEQ ID NO: 115, and the VL comprises the LCDRs 1-3 of a VL having the amino acid sequence set forth in SEQ ID NO: 131; or
(14) the VH comprises the HCDRs 1-3 of a VH having the amino acid sequence set forth in SEQ ID NO: 116, and the VL comprises the LCDRs 1-3 of a VL having the amino acid sequence set forth in SEQ ID NO: 132.

In a second aspect, the invention provides an antibody that specifically binds to CD200R1, or an antigen binding fragment thereof, wherein the antibody comprises a light chain variable region (VL) and a heavy chain variable region (VH), and wherein
(1) the VL comprises LCDR1-3 having the amino acid sequences set forth in SEQ ID NOs: 67, 80, 99 respectively or LCDRs 1-3 having amino acid sequences each of which differ by one or two amino acid residues as the sequences set forth in SEQ ID NOs: 67, 80, 99 respectively, and the VH comprises HCDRs 1-3 having the amino acid sequences set forth in SEQ ID NOs: 11, 32, 46 respectively or HCDRs 1-3 having amino acid sequences each of which differ by one or two amino acid residues as the sequences set forth in SEQ ID NOs: 11, 32, 46 respectively; or
(2) the VL comprises LCDR1-3 having the amino acid sequences set forth in SEQ ID NOs: 62, 76, 92 respectively or LCDRs 1-3 having amino acid sequences each of which differ by one or two amino acid residues as the sequences set forth in SEQ ID NOs: 62, 76, 92 respectively, and the VH comprises HCDRs 1-3 having the amino acid sequences set forth in SEQ ID NOs: 11, 26, 46 respectively or HCDRs 1-3 having amino acid sequences each of which differ by one or two amino acid residues as the sequences set forth in SEQ ID NOs: 11, 26, 46 respectively; or
(3) the VL comprises LCDR1-3 having the amino acid sequences set forth in SEQ ID NOs: 66, 80, 92 respectively or LCDRs 1-3 having amino acid sequences each of which differ by one or two amino acid residues as the sequences set forth in SEQ ID NOs: 66, 80, 92 respectively, and the VH comprises HCDRs 1-3 having the amino acid sequences set forth in SEQ ID NOs: 11, 32, 46 respectively or HCDRs 1-3 having amino acid sequences each of which differ by one or two amino acid residues as the sequences set forth in SEQ ID NOs: 11, 32, 46 respectively; or
(4) the VL comprises LCDR1-3 having the amino acid sequences set forth in SEQ ID NOs: 66, 81, 92 respectively or LCDRs 1-3 having amino acid sequences each of which differ by one or two amino acid residues as the sequences set forth in SEQ ID NOs: 66, 81, 92 respectively, and the VH comprises HCDRs 1-3 having the amino acid sequences set forth in SEQ ID NOs: 11, 33, 46 respectively or HCDRs 1-3 having amino acid sequences each of which differ by one or two amino acid residues as the sequences set forth in SEQ ID NOs: 11, 33, 46 respectively; or
(5) the VL comprises LCDR1-3 having the amino acid sequences set forth in SEQ ID NOs: 67, 76, 99 respectively or LCDRs 1-3 having amino acid sequences each of which differ by one or two amino acid residues as the sequences set forth in SEQ ID NOs: 67, 76, 99 respectively, and the VH comprises HCDRs 1-3 having the amino acid sequences set forth in SEQ ID NOs: 14, 32, 46 respectively or HCDRs 1-3 having amino acid sequences each of which differ by one or two amino acid residues as the sequences set forth in SEQ ID NOs: 14, 32, 46 respectively; or
(6) the VL comprises LCDR1-3 having the amino acid sequences set forth in SEQ ID NOs: 66, 80, 100 respectively or LCDRs 1-3 having amino acid sequences each of which differ by one or two amino acid residues as the sequences set forth in SEQ ID NOs: 66, 80, 100 respectively, and the VH comprises HCDRs 1-3 having the amino acid sequences set forth in SEQ ID NOs: 14, 33, 46 respectively or HCDRs 1-3 having amino acid sequences each of which differ by one or two amino acid residues as the sequences set forth in SEQ ID NOs: 14, 33, 46 respectively; or
(7) the VL comprises LCDRs 1-3 having the amino acid sequences set forth in SEQ ID NOs: 62, 76, 91 respectively or LCDRs 1-3 having amino acid sequences each of which differ by one or two amino acid residues as the sequences set forth in SEQ ID NOs: 62, 76, 91 respectively, and the VH comprises HCDRs 1-3 having the amino acid sequences set forth in SEQ ID NOs: 10, 25, 44 respectively or HCDRs 1-3 having amino acid sequences each of which differ by one or two amino acid residues as the sequences set forth in SEQ ID NOs: 10, 25, 44 respectively; or
(8) the VL comprises LCDR1-3 having the amino acid sequences set forth in SEQ ID NOs: 62, 76, 92 respectively or LCDRs 1-3 having amino acid sequences each of which differ by one or two amino acid residues as the sequences set forth in SEQ ID NOs: 62, 76, 92 respectively, and the VH comprises HCDRs 1-3 having the amino acid sequences set forth in SEQ ID NOs: 11, 26, 45 respectively or HCDRs 1-3 having amino acid sequences each of which differ by one or two amino acid residues as the sequences set forth in SEQ ID NOs: 11, 26, 45 respectively; or
(9) the VL comprises LCDR1-3 having the amino acid sequences set forth in SEQ ID NOs: 62, 76, 93 respectively or LCDRs 1-3 having amino acid sequences each of which differ by one or two amino acid residues as the sequences set forth in SEQ ID NOs: 62, 76, 93 respectively, and the VH comprises HCDRs 1-3 having the amino acid sequences set forth in SEQ ID NOs: 11, 25, 45 respectively or HCDRs 1-3 having amino acid sequences each of which differ by one or two amino acid residues as the sequences set forth in SEQ ID NOs: 11, 25, 45 respectively; or
(10) the VL comprises LCDR1-3 having the amino acid sequences set forth in SEQ ID NOs: 65, 79, 96 respectively or LCDRs 1-3 having amino acid sequences each of which differ by one or two amino acid residues as the sequences set forth in SEQ ID NOs: 65, 79, 96 respectively, and the VH comprises HCDRs 1-3 having the amino acid sequences set forth in SEQ ID NOs: 11, 29, 49 respectively or HCDRs 1-3 having amino acid sequences each of which differ by one or two amino acid residues as the sequences set forth in SEQ ID NOs: 11, 29, 49 respectively; or
(11) the VL comprises LCDR1-3 having the amino acid sequences set forth in SEQ ID NOs: 65, 79, 97 respectively or LCDRs 1-3 having amino acid sequences each of which differ by one or two amino acid residues as the sequences set forth in SEQ ID NOs: 65, 79, 97 respectively, and the VH comprises HCDRs 1-3 having the amino acid sequences set forth in SEQ ID NOs: 11, 30, 49 respectively or HCDRs 1-3 having amino acid sequences each of which differ by one or two amino acid residues as the sequences set forth in SEQ ID NOs: 11, 30, 49 respectively; or
(12) the VL comprises LCDR1-3 having the amino acid sequences set forth in SEQ ID NOs: 65, 79, 98 respectively or LCDRs 1-3 having amino acid sequences each of which differ by one or two amino acid residues as the sequences set forth in SEQ ID NOs: 65, 79, 98 respectively, and the VH comprises HCDRs 1-3 having the amino acid sequences set forth in SEQ ID NOs: 11, 31, 50 respectively or HCDRs 1-3 having amino acid sequences each of which differ by one or two amino acid residues as the sequences set forth in SEQ ID NOs: 11, 31, 50 respectively; or
(13) the VL comprises LCDR1-3 having the amino acid sequences set forth in SEQ ID NOs: 65, 79, 97 respectively or LCDRs 1-3 having amino acid sequences each of which differ by one or two amino acid residues as the sequences set forth in SEQ ID NOs: 65, 79, 97 respectively, and the VH comprises HCDRs 1-3 having the amino acid sequences set forth in SEQ ID NOs: 11, 30, 49 respectively or HCDRs 1-3 having amino acid sequences each of which differ by one or two amino acid residues as the sequences set forth in SEQ ID NOs: 11, 30, 49 respectively; or
(14) the VL comprises LCDR1-3 having the amino acid sequences set forth in SEQ ID NOs: 65, 79, 98 respectively or LCDRs 1-3 having amino acid sequences each of which differ by one or two amino acid residues as the sequences set forth in SEQ ID NOs: 65, 79, 98 respectively, and the VH comprises HCDRs 1-3 having the amino acid sequences set forth in SEQ ID NOs: 11, 31, 50 respectively or HCDRs 1-3 having amino acid sequences each of which differ by one or two amino acid residues as the sequences set forth in SEQ ID NOs: 11, 31, 50 respectively.

In some embodiments, the invention provides an antibody that specifically binds to CD200R1, or an antigen binding fragment thereof, wherein the antibody comprises a light chain variable region (VL) and a heavy chain variable region (VH), and wherein
(1) the VL comprises the amino acid sequence having at least 80%, at least 85%, at least 90%, at least 95%, at least 98%, at least 99%, or 100% sequence identity to SEQ ID No:136, and the VH comprises the amino acid sequence having at least 80%, at least 85%, at least 90%, at least 95%, at least 98%, at least 99%, or 100% sequence identity to SEQ ID No:117; or
(2) the VL comprises the amino acid sequence having at least 80%, at least 85%, at least 90%, at least 95%, at least 98%, at least 99%, or 100% sequence identity to SEQ ID No:124, and the VH comprises the amino acid sequence having at least 80%, at least 85%, at least 90%, at least 95%, at least 98%, at least 99%, or 100% sequence identity to SEQ ID No:111; or
(3) the VL comprises the amino acid sequence having at least 80%, at least 85%, at least 90%, at least 95%, at least 98%, at least 99%, or 100% sequence identity to SEQ ID No:133, and the VH comprises the amino acid sequence having at least 80%, at least 85%, at least 90%, at least 95%, at least 98%, at least 99%, or 100% sequence identity to SEQ ID No:117; or
(4) the VL comprises the amino acid sequence having at least 80%, at least 85%, at least 90%, at least 95%, at least 98%, at least 99%, or 100% sequence identity to SEQ ID No:134, and the VH comprises the amino acid sequence having at least 80%, at least 85%, at least 90%, at least 95%, at least 98%, at least 99%, or 100% sequence identity to SEQ ID No:118; or
(5) the VL comprises the amino acid sequence having at least 80%, at least 85%, at least 90%, at least 95%, at least 98%, at least 99%, or 100% sequence identity to SEQ ID No:135, and the VH comprises the amino acid sequence having at least 80%, at least 85%, at least 90%, at least 95%, at least 98%, at least 99%, or 100% sequence identity to SEQ ID No:119; or
(6) the VL comprises the amino acid sequence having at least 80%, at least 85%, at least 90%, at least 95%, at least 98%, at least 99%, or 100% sequence identity to SEQ ID No 137, and the VH comprises the amino acid sequence having at least 80%, at least 85%, at least 90%, at least 95%, at least 98%, at least 99%, or 100% sequence identity to SEQ ID No 120; or
(7) the VL comprises the amino acid sequence having at least 80%, at least 85%, at least 90%, at least 95%, at least 98%, at least 99%, or 100% sequence identity to SEQ ID No:123, and the VH comprises the amino acid sequence having at least 80%, at least 85%, at least 90%, at least 95%, at least 98%, at least 99%, or 100% sequence identity to SEQ ID No:108; or
(8) the VL comprises the amino acid sequence having at least 80%, at least 85%, at least 90%, at least 95%, at least 98%, at least 99%, or 100% sequence identity to SEQ ID No:124, and the VH comprises the amino acid sequence having at least 80%, at least 85%, at least 90%, at least 95%, at least 98%, at least 99%, or 100% sequence identity to SEQ ID No:109; or
(9) the VL comprises the amino acid sequence having at least 80%, at least 85%, at least 90%, at least 95%, at least 98%, at least 99%, or 100% sequence identity to SEQ ID No:125, and the VH comprises the amino acid sequence having at least 80%, at least 85%, at least 90%, at least 95%, at least 98%, at least 99%, or 100% sequence identity to SEQ ID No:110; or
(10) the VL comprises the amino acid sequence having at least 80%, at least 85%, at least 90%, at least 95%, at least 98%, at least 99%, or 100% sequence identity to SEQ ID No:128, and the VH comprises the amino acid sequence having at least 80%, at least 85%, at least 90%, at least 95%, at least 98%, at least 99%, or 100% sequence identity to SEQ ID No:114; or
(11) the VL comprises the amino acid sequence having at least 80%, at least 85%, at least 90%, at least 95%, at least 98%, at least 99%, or 100% sequence identity to SEQ ID No:129, and the VH comprises the amino acid sequence having at least 80%, at least 85%, at least 90%, at least 95%, at least 98%, at least 99%, or 100% sequence identity to SEQ ID No:115; or
(12) the VL comprises the amino acid sequence having at least 80%, at least 85%, at least 90%, at least 95%, at least 98%, at least 99%, or 100% sequence identity to SEQ ID No:130, and the VH comprises the amino acid sequence having at least 80%, at least 85%, at least 90%, at least 95%, at least 98%, at least 99%, or 100% sequence identity to SEQ ID No:116; or
(13) the VL comprises the amino acid sequence having at least 80%, at least 85%, at least 90%, at least 95%, at least 98%, at least 99%, or 100% sequence identity to SEQ ID No:131, and the VH comprises the amino acid sequence having at least 80%, at least 85%, at least 90%, at least 95%, at least 98%, at least 99%, or 100% sequence identity to SEQ ID No:115; or
(14) the VL comprises the amino acid sequence having at least 80%, at least 85%, at least 90%, at least 95%, at least 98%, at least 99%, or 100% sequence identity to SEQ ID No:132, and the VH comprises the amino acid sequence having at least 80%, at least 85%, at least 90%, at least 95%, at least 98%, at least 99%, or 100% sequence identity to SEQ ID No:116.

In some embodiments, the VL comprises a functional variant of the amino acid sequence as set forth in any one of SEQ ID NOs: 123-125, 128-137 formed by insertion, deletion and/or substitution of one or more amino acid(s) therein, provided that the functional variant retains the ability of binding to CD200R1. In some embodiments, the VH comprises a functional variant of the amino acid sequence as set forth in any one of SEQ ID NO: 108-111, 114-120 formed by insertion, deletion and/or substitution of one or more amino acid(s) therein, provided that the functional variant retains the ability of binding to CD200R1.

The functional variant comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the amino acid sequence of the parent polypeptide. For example, the functional variant of any one of SEQ ID NOs: 123-125, 128-137 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one of SEQ ID NOs: 123-125, 128-137, respectively. For example, the functional variant of any one of SEQ ID NO: 108-111, 114-120 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one of SEQ ID NO: 108-111, 114-120.

In some embodiments, the functional variant of any one of SEQ ID NOs: 123-125, 128-137 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one of SEQ ID NOs: 123-125, 128-137 and formed by insertion, deletion and/or substitution of one or more amino acid(s) in any one of SEQ ID NOs: 123-125, 128-137. In some embodiments, the functional variant of any one of SEQ ID NO: 108-111, 114-120 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one of SEQ ID NO: 108-111, 114-120 and formed by insertion, deletion and/or substitution of one or more amino acid(s) in any one of SEQ ID NO: 108-111, 114-120.

In the context of the functional variant, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-2%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (e) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In a preferred embodiment, the VL comprises an amino acid sequence as set forth in SEQ ID NO: 136 and the VH comprises an amino acid sequence as set forth in SEQ ID NO: 117; or the VL comprises an amino acid sequence as set forth in SEQ ID NO: 124 and the VH comprises an amino acid sequence as set forth in SEQ ID NO: 111; or the VL comprises an amino acid sequence as set forth in SEQ ID NO: 133 and the VH comprises an amino acid sequence as set forth in SEQ ID NO: 117; or the VL comprises an amino acid sequence as set forth in SEQ ID NO: 134 and the VH comprises an amino acid sequence as set forth in SEQ ID NO: 118; or the VL comprises an amino acid sequence as set forth in SEQ ID NO: 135 and the VH comprises an amino acid sequence as set forth in SEQ ID NO: 119; or the VL comprises an amino acid sequence as set forth in SEQ ID NO: 137 and the VH comprises an amino acid sequence as set forth in SEQ ID NO: 120; or the VL comprises an amino acid sequence as set forth in SEQ ID NO: 123 and the VH comprises an amino acid sequence as set forth in SEQ ID NO: 108; or the VL comprises an amino acid sequence as set forth in SEQ ID NO: 124 and the VH comprises an amino acid sequence as set forth in SEQ ID NO:109; or the VL comprises an amino acid sequence as set forth in SEQ ID NO: 125 and the VH comprises an amino acid sequence as set forth in SEQ ID NO: 110; or the VL comprises an amino acid sequence as set forth in SEQ ID NO: 128 and the VH comprises an amino acid sequence as set forth in SEQ ID NO: 114; or the VL comprises an amino acid sequence as set forth in SEQ ID NO: 129 and the VH comprises an amino acid sequence as set forth in SEQ ID NO: 115; or the VL comprises an amino acid sequence as set forth in SEQ ID NO: 130 and the VH comprises an amino acid sequence as set forth in SEQ ID NO: 116; or the VL comprises an amino acid sequence as set forth in SEQ ID NO: 131 and the VH comprises an amino acid sequence as set forth in SEQ ID NO: 115; or the VL comprises an amino acid sequence as set forth in SEQ ID NO: 132 and the VH comprises an amino acid sequence as set forth in SEQ ID NO: 116.

Based on the amino acid sequence of heavy chain constant regions of the antibody, a immunoglobulin molecule can be divided into five classes (isotypes): IgA, IgD, IgE, IgG, and IgM, and can be further divided into different subtypes, such as IgG1, IgG2, IgG3, IgG4, IgA1, IgA2, etc. The light chain of the antibody can be classified as a lambda (k) chain or a kappa (κ) chain, based on the amino acid sequence of the light chain. The antibodies disclosed herein can be of any classes or subtypes above.

In some embodiments, the antibody can be of an isotype selected from the group consisting of IgG, IgA, IgM, IgE and IgD. In some embodiments, the antibody can be of a subtype selected from the group consisting of IgG1, IgG2, IgG3, and IgG4. In a preferred embodiment, the antibody is an IgG1 antibody.

The antibody disclosed herein can be an intact antibody or the antigen binding fragment thereof. The antigen binding fragment can be any fragments of the antibody that retain the ability to specifically bind to CD200R1. Examples of antigen binding fragments include but are not limited to a Fab fragment; a F(ab′)2 fragment; a Fab′ fragment; a Fd fragment; a Fd′ fragment; a Fv fragment; a scFv fragment; a dAb fragment; an isolated complementarity determining region (CDR); a nanobody; a linear antibody comprising a pair of tandem Fd segments (VH-CH1-VH-CH1), and a modified version of any of the foregoing fragments, which retains antigen binding activity.

In some embodiments, the antigen binding fragment can be selected from the group consisting of Fab, Fab′, F(ab′)₂, Fv, scFv, and ds-scFv. In a preferred embodiment, the antigen binding fragment is Fab or scFv.

In some embodiments, the antibody can be a monoclonal antibody. In some embodiments, the antibody comprises a heavy chain (HC) and a light chain (LC), and wherein (1) the LC comprises the amino acid sequence having at least 80%, at least 85%, at least 90%, at least 95%, at least 98%, at least 99%, or 100% sequence identity to SEQ ID No: 170, and the HC comprises the amino acid sequence having at least 80%, at least 85%, at least 90%, at least 95%, at least 98%, at least 99%, or 100% sequence identity to SEQ ID No: 151; or
(2) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 143; or
(3) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 147; or
(4) the LC comprises the amino acid sequence having at least 80%, at least 85%, at least 90%, at least 95%, at least 98%, at least 99%, or 100% sequence identity to SEQ ID No: 167, and the HC comprises the amino acid sequence having at least 80%, at least 85%, at least 90%, at least 95%, at least 98%, at least 99%, or 100% sequence identity to SEQ ID No: 151; or
(5) the LC comprises the amino acid sequence having at least 80%, at least 85%, at least 90%, at least 95%, at least 98%, at least 99%, or 100% sequence identity to SEQ ID No: 168, and the HC comprises the amino acid sequence having at least 80%, at least 85%, at least 90%, at least 95%, at least 98%, at least 99%, or 100% sequence identity to SEQ ID No: 152; or
(6) the LC comprises the amino acid sequence having at least 80%, at least 85%, at least 90%, at least 95%, at least 98%, at least 99%, or 100% sequence identity to SEQ ID No: 169, and the HC comprises the amino acid sequence having at least 80%, at least 85%, at least 90%, at least 95%, at least 98%, at least 99%, or 100% sequence identity to SEQ ID No: 153; or
(7) the LC comprises the amino acid sequence having at least 80%, at least 85%, at least 90%, at least 95%, at least 98%, at least 99%, or 100% sequence identity to SEQ ID No: 171, and the HC comprises the amino acid sequence having at least 80%, at least 85%, at least 90%, at least 95%, at least 98%, at least 99%, or 100% sequence identity to SEQ ID No: 154; or
(8) the LC comprises the amino acid sequence having at least 80%, at least 85%, at least 90%, at least 95%, at least 98%, at least 99%, or 100% sequence identity to SEQ ID No: 157, and the HC comprises the amino acid sequence having at least 80%, at least 85%, at least 90%, at least 95%, at least 98%, at least 99%, or 100% sequence identity to SEQ ID No: 140; or
(9) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 141; or
(10) the LC comprises the amino acid sequence having at least 80%, at least 85%, at least 90%, at least 95%, at least 98%, at least 99%, or 100% sequence identity to SEQ ID No: 159, and the HC comprises the amino acid sequence having at least 80%, at least 85%, at least 90%, at least 95%, at least 98%, at least 99%, or 100% sequence identity to SEQ ID No: 142; or
(11) the LC comprises the amino acid sequence having at least 80%, at least 85%, at least 90%, at least 95%, at least 98%, at least 99%, or 100% sequence identity to SEQ ID No: 162, and the HC comprises the amino acid sequence having at least 80%, at least 85%, at least 90%, at least 95%, at least 98%, at least 99%, or 100% sequence identity to SEQ ID No: 146; or
(12) the LC comprises the amino acid sequence having at least 80%, at least 85%, at least 90%, at least 95%, at least 98%, at least 99%, or 100% sequence identity to SEQ ID No: 162, and the HC comprises the amino acid sequence having at least 80%, at least 85%, at least 90%, at least 95%, at least 98%, at least 99%, or 100% sequence identity to SEQ ID No: 148; or
(13) the LC comprises the amino acid sequence having at least 80%, at least 85%, at least 90%, at least 95%, at least 98%, at least 99%, or 100% sequence identity to SEQ ID No: 163, and the HC comprises the amino acid sequence having at least 80%, at least 85%, at least 90%, at least 95%, at least 98%, at least 99%, or 100% sequence identity to SEQ ID No: 149; or
(14) the LC comprises the amino acid sequence having at least 80%, at least 85%, at least 90%, at least 95%, at least 98%, at least 99%, or 100% sequence identity to SEQ ID No: 164, and the HC comprises the amino acid sequence having at least 80%, at least 85%, at least 90%, at least 95%, at least 98%, at least 99%, or 100% sequence identity to SEQ ID No: 150; or
(15) the LC comprises the amino acid sequence having at least 80%, at least 85%, at least 90%, at least 95%, at least 98%, at least 99%, or 100% sequence identity to SEQ ID No: 165, and the HC comprises the amino acid sequence having at least 80%, at least 85%, at least 90%, at least 95%, at least 98%, at least 99%, or 100% sequence identity to SEQ ID No: 149; or
(16) the LC comprises the amino acid sequence having at least 80%, at least 85%, at least 90%, at least 95%, at least 98%, at least 99%, or 100% sequence identity to SEQ ID No: 166, and the HC comprises the amino acid sequence having at least 80%, at least 85%, at least 90%, at least 95%, at least 98%, at least 99%, or 100% sequence identity to SEQ ID No: 150.

In some embodiments, the light chain comprises a functional variant of the amino acid sequence as set forth in any one of formed by insertion, deletion and/or substitution of one or more amino acid(s) therein, provided that the functional variant retains the ability of binding to any one of SEQ ID NOs: 157-159, and 162-171. In some embodiments, the heavy chain comprises a functional variant of the amino acid sequence as set forth in any one SEQ ID NOs: 140-143, and 146-154 formed by insertion, deletion and/or substitution of one or more amino acid(s) therein, provided that the functional variant retains the ability of binding to CD200R1.

For example, the functional variant of any one of SEQ ID NOs: 157-159, and 162-171 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one of SEQ ID NOs: 157-159, and 162-171, respectively. For example, the functional variant of any one SEQ ID NOs: 140-143, and 146-154 comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one SEQ ID NOs: 140-143, and 146-154 respectively.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-50, preferably 1-20, more preferably 1-10, still more preferably 1-5. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3 and/or FR4; and/or constant regions, e.g., CL, CH1, CH2 and/or CH3.

In some embodiments, the substitution of one or more amino acid(s) can be conservative substitution of one or more amino acid(s). Examples of conservative substitutions are as described above.

In a preferred embodiment, the light chain comprises an amino acid sequence as set forth in SEQ ID NO: 170 and the heavy chain comprises an amino acid sequence as set forth in SEQ ID NO: 151; or the light chain comprises an amino acid sequence as set forth in SEQ ID NO: 158 and the heavy chain comprises an amino acid sequence as set forth in SEQ ID NO: 143; or the light chain comprises an amino acid sequence as set forth in SEQ ID NO: 158 and the heavy chain comprises an amino acid sequence as set forth in SEQ ID NO: 147; or the light chain comprises an amino acid sequence as set forth in SEQ ID NO: 167 and the heavy chain comprises an amino acid sequence as set forth in SEQ ID NO: 151; or the light chain comprises an amino acid sequence as set forth in SEQ ID NO: 168 and the heavy chain comprises an amino acid sequence as set forth in SEQ ID NO: 152; or the light chain comprises an amino acid sequence as set forth in SEQ ID NO: 169 and the heavy chain comprises an amino acid sequence as set forth in SEQ ID NO: 153; or the light chain comprises an amino acid sequence as set forth in SEQ ID NO: 171 and the heavy chain comprises an amino acid sequence as set forth in SEQ ID NO: 154; or the light chain comprises an amino acid sequence as set forth in SEQ ID NO: 157 and the heavy chain comprises an amino acid sequence as set forth in SEQ ID NO: 140; or the light chain comprises an amino acid sequence as set forth in SEQ ID NO: 158 and the heavy chain comprises an amino acid sequence as set forth in SEQ ID NO: 141; or the light chain comprises an amino acid sequence as set forth in SEQ ID NO: 159 and the heavy chain comprises an amino acid sequence as set forth in SEQ ID NO:142; or the light chain comprises an amino acid sequence as set forth in SEQ ID NO: 162 and the heavy chain comprises an amino acid sequence as set forth in SEQ ID NO: 146; or the light chain comprises an amino acid sequence as set forth in SEQ ID NO: 162 and the heavy chain comprises an amino acid sequence as set forth in SEQ ID NO: 148; or the light chain comprises an amino acid sequence as set forth in SEQ ID NO: 163 and the heavy chain comprises an amino acid sequence as set forth in SEQ ID NO: 149; or the light chain comprises an amino acid sequence as set forth in SEQ ID NO: 164 and the heavy chain comprises an amino acid sequence as set forth in SEQ ID NO: 150; or the light chain comprises an amino acid sequence as set forth in SEQ ID NO: 165 and the heavy chain comprises an amino acid sequence as set forth in SEQ ID NO: 149; or the light chain comprises an amino acid sequence as set forth in SEQ ID NO: 166 and the heavy chain comprises an amino acid sequence as set forth in SEQ ID NO: 150.

In some embodiments, the antibody comprises an Fc region. In some embodiments, the Fc region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fc region is human IgG1 Fc.

In one embodiment, the Fc region is effector-function-deficient. For example, the Fc region may be of an IgG1 isotype, or a non-IgG1 type, e.g. IgG2, IgG3 or IgG4, which has been mutated such that the ability to mediate effector functions, such as ADCC, has been reduced or even eliminated. Such mutations have e.g. been described in Dall'Acqua W F et al., J Immunol. 177(2):1129-1138 (2006) and Hezareh M, J Virol.; 75(24):12161-12168 (2001). In some embodiments, the Fc region of the antibody comprises a wild type IgG1 Fc with L234A, L235A and G237A mutations.

In some embodiments, the antibody is mutated at one or more post-translational modifications sites. In one embodiment, the Fc region comprises a mutation removing the acceptor site for Asn-linked glycosylation or is otherwise manipulated to eliminate the effector function of the antibody.

In some embodiments, the antibody is a bispecific or a multi-specific antibody. In some embodiments, the antibody is a bispecific antibody which further comprises a second antigen binding region binding to a second antigen. In some embodiments, the second antigen can be a tumor associated antigen, an immune checkpoint molecule or an immune cell antigen.

The anti-CD200R1 antibody of the invention is a naturally occurring antibody having a binding activity to human CD200R1 and Cynomolgus macaques CD200R1. The Fc-engineered antibody has no ADCC effect and effectively block CD200-CD200R1 interaction, inhibits CD200-induced CD200R1 signaling pathway and activates immune responses; the antibody also shows no agonistic activity, and therefore is a potential antibody molecule suitable for antagonistic antibody development.

### Bispecific Antibody

In a second aspect, the present application provides a bispecific or a multi-specific antibody. In some embodiments, the antibody is a bispecific antibody which further comprises a second antigen binding region binding to a second antigen. In some embodiments, the second antigen can be a tumor associated antigen, an immune checkpoint molecule or an immune cell antigen.

Many tumor associated antigens associated with specific cancers have been identified in the art. In some embodiments, tumor-associated antigens are antigens that can potentially stimulate an obvious tumor-specific immune response. Some of these antigens are encoded by normal cells, but not necessarily expressed by normal cells. These antigens can be characterized as those that are usually silent (i.e., not expressed) in normal cells, those that are expressed only during certain stages of differentiation, and those that are expressed over time, such as embryonic and fetal antigens. Other cancer antigens are encoded by mutant cell genes such as oncogenes (e.g. activated ras oncogene), suppressor genes (e.g. mutant p53), and fusion proteins produced by internal deletions or chromosomal translocations. Other cancer antigens can be encoded by viral genes, such as those carried on RNA and DNA tumor viruses. Many other tumor associated antigens and antibodies against them are known and/or commercially available, and can also be produced by those skilled in the art.

Examples of tumor associated antigens include but are not limited to 5T4, alphafetoprotein, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFR, EGFRvIII, epithelial tumor antigen, ERBB2, FLT3, folate binding protein, GD2, GD3, HIV-1 envelope glycoprotein gp41, HIV-1 envelope glycoprotein gpl20, melanoma-associated antigen, CD200R1, MUC-1, mutated p53, mutated ras, ROR1, VEGFR2, and combinations thereof.

In some embodiments, the second antigen is a T-cell antigen. In some embodiments, the T-cell antigen can be selected from the group consisting of T cell receptor (TCR), CD3, CD4, CD8, CD16, CD25, CD28, CD44, CD62L, CD69, ICOS, 41-BB (CD137), and NKG2D or any combination thereof. 8, CD44, CD62L, CD69, ICOS, 41-BB (CD137), and NKG2D or any combination thereof.

In some embodiments, the second antigen is an immune checkpoint molecule. In some embodiments, the immune checkpoint molecule can be selected from the group consisting of PD-1, PD-L1, CTLA-4, and the like.

In some embodiments, the bispecific antibody comprises a single polypeptide chain comprising the first antigen binding region and the second antigen binding region, and optionally an Fc region.

The Fc region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fc region is human IgG1 Fc.

In one embodiment, the Fc region is effector-function-deficient. For example, the Fc region may be of an IgG1 isotype, or a non-IgG1 type, e.g. IgG2, IgG3 or IgG4, which has been mutated such that the ability to mediate effector functions, such as ADCC, has been reduced or even eliminated. Such mutations have e.g. been described in Dall'Acqua W F et al., J Immunol. 177(2):1129-1138 (2006) and Hezareh M, J Virol.; 75(24):12161-12168 (2001).

In one embodiment, the Fc region comprises a mutation removing the acceptor site for Asn-linked glycosylation or is otherwise manipulated to change the glycosylation properties. For example, in an IgG1 Fc region, an N297Q mutation can be used to remove an Asn-linked glycosylation site. Accordingly, in a specific embodiment, Fc region comprise an IgG1 wildtype sequence with an N297Q mutation. For example, in an IgG1 Fc region, an N297Q mutation can be used to remove an Asn-linked glycosylation site. Accordingly, in a specific embodiment, Fc region comprise an IgG1 wildtype sequence with an N297Q mutation.

In a further embodiment, the Fc region is glyco-engineered to reduce fucose and thus enhance ADCC, e.g. by addition of compounds to the culture media during antibody production as described in US2009317869 or as described in van Berkel et al. (2010) Biotechnol. Bioeng. 105:350 or by using FUT8 knockout cells, e.g. as described in Yamane-Ohnuki et al. (2004) Biotechnol. Bioeng 87:614. ADCC may alternatively be optimized using the method described by Umana et al. (1999) Nature Biotech 17:176. In a further embodiment, the Fc region has been engineered to enhance complement activation, e.g. as described in Natsume et al. (2009) Cancer Sci. 100:2411.

In some embodiments, the Fc region comprises modifications or mutations that can inhibit Fc homodimerization. In some embodiments, the Fc region comprises a variant of a human IgG1 Fc wild type sequence. The variant can comprise amino acid substitutions at positions T366 and Y407 of human IgG1 (Kabat numbering). Preferably, T366 is substituted with L (Leucine). Preferably, Y407 is substituted with I(Isoleucine), F(Phenylalanine), L(Leucine), M(Methionine), H(Histidine), K(Lysine), S(Serine), Q(Glutamine), T(Threonine), W(Tryptophan), A(Alanine), G(Glycine) or N (Asparagine). More preferably, Y407 is substituted with H. In one embodiment, T366 is substituted with L, and Y407 is substituted with H.

In some embodiments, the Fc region can be a monomeric human IgG1 Fc. as described in PCT application No. PCT/US2018/016524, which is incorporated herein by reference in its entirety.

In some embodiments, the bispecific antibody comprises a first polypeptide chain comprising the VL of the first antigen binding region and the VL of the second antigen binding region, and optionally an Fc region; and a second polypeptide chain comprising the VH of the first antigen binding region and the VH of the second antigen binding region, and optionally an Fc region. The Fc region can be those as describe above.

In some embodiments, the first polypeptide chain further comprises a light chain constant region (CL). In some embodiments, the first polypeptide chain comprises a monomeric human IgG1 Fc as described above. In some embodiments, the first polypeptide chain comprises, from N-terminal to C-terminal: the VL of the first antigen binding region, the VL of the second antigen binding region, CL and Fc.

In some embodiments, the second polypeptide chain further comprises a heavy chain constant region (CH), e.g., CH1. In some embodiments, the first polypeptide chain comprises a monomeric human IgG1 Fc as described above. In some embodiments, the second polypeptide chain comprises, from N-terminal to C-terminal: the VH of the first antigen binding region, the VH of the second antigen binding region, CH1 and Fc.

### Nucleic Acid

In a third aspect, the invention provides a nucleic acid comprising a nucleotide sequence encoding the anti-CD200R1 antibody or the antigen binding fragment thereof disclosed herein or the bispecific antibody or the antigen binding fragment thereof disclosed herein. The term “polynucleotide” or “nucleic acid” includes both single-stranded and double-stranded nucleotide polymers. The nucleotides comprising nucleic acid can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate. For example, the invention provides nucleic acid molecules encoding any one of the heavy chain variable region sequences disclosed herein. The invention also provides nucleic acid molecules that are at least 90%, at least 95%, at least 98% or at least 99% identical to nucleic acids encoding any one of the heavy chain variable region sequences disclosed herein.

For example, the invention provides nucleic acid molecules encoding any one of the light chain variable region sequences disclosed herein. The invention also provides nucleic acid molecules that are at least 90%, at least 95%, at least 98% or at least 99% identical to nucleic acids encoding any one of the light chain variable region sequences disclosed herein.

For example, the invention provides nucleic acid molecules encoding: (i) any one of the heavy chain variable region sequences disclosed herein and (ii) any one of the light chain variable region sequences disclosed herein. The invention also provides nucleic acid molecules that are at least 90%, at least 95%, at least 98% or at least 99% identical to nucleic acids encoding: (i) any one of the heavy chain variable region sequences disclosed herein and (ii) any one of the light chain variable region sequences disclosed herein. For example, the invention provides nucleic acid molecules encoding a heavy chain variable region sequence that comprises the CDR sequences of any one of the heavy chain variable region sequences disclosed herein.

In some embodiments, the invention provides nucleic acid molecules encoding a heavy chain variable region sequence that comprises any one of the groups of three CDR sequences disclosed herein.

The invention also provides nucleic acid molecules that encode a heavy chain variable region sequence that comprises CDR sequences that are at least 90%, at least 95%, at least 98% or at least 99% identical to the CDR sequences of any one of the heavy chain variable region sequences disclosed herein.

In some embodiments, the invention provides nucleic acid molecules that encode a heavy chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein. For example, the invention provides nucleic acid molecules encoding a light chain variable region sequence that comprises the CDR sequences of any one of the light chain variable region sequences disclosed herein.

In some embodiments, the invention provides nucleic acid molecules encoding a light chain variable region sequence that comprises any one of the groups of three CDR sequences disclosed herein.

The invention also provides nucleic acid molecules that encode a light chain variable region sequence that comprises CDR sequences that are at least 90%, at least 95%, at least 98% or at least 99% identical to the CDR sequences of any one of the light chain variable region sequences disclosed herein.

In some embodiments, the invention provides nucleic acid molecules that encode a light chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein.

For example, the invention provides nucleic acid molecules encoding: (i) a heavy chain variable region sequence that comprises the CDR sequences of any one of the heavy chain variable region sequences disclosed herein and (ii) a light chain variable region sequence that comprises the CDR sequences of any one of the light chain variable region sequences disclosed herein. In some embodiments, the invention provides nucleic acid molecules encoding (i) a heavy chain variable region sequence that comprises any one of the groups of three CDR sequences disclosed herein and (ii) a light chain variable region sequence that comprises any one of the groups of three CDR sequences disclosed herein. The invention also provides nucleic acid molecules that encode: (i) a heavy chain variable region sequence that comprises CDR sequences that are at least 90%, at least 95%, at least 98% or at least 99% identical to the CDR sequences of any one of the heavy chain variable region sequences disclosed herein and (ii) a light chain variable region sequence that comprises CDR sequences that are at least 90%, at least 95%, at least 98% or at least 99% identical to the CDR sequences of any one of the light chain variable region sequences disclosed herein. In some embodiments, the invention provides nucleic acid molecules that encode (i) a heavy chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein and (ii) a light chain variable region sequence that comprises CDR1, CDR2 and CDR3 sequences that are at least 90%, at least 95%, at least 98% or at least 99% identical to the CDR1, CDR2 and CDR3, respectively, of any one of the groups of three CDR sequences disclosed herein.

In some embodiments, the nucleic acid is ribonucleic acid (RNA) or deoxyribonucleic acid (DNA). In some embodiments, the invention provides a ribonucleic acid (RNA) comprising a nucleotide sequence encoding the anti-CD200R1 antibody or the antigen binding fragment thereof disclosed herein or the bispecific antibody or the antigen binding fragment thereof disclosed herein. In some embodiments, the invention provides a deoxyribonucleic acid (DNA) comprising a deoxynucleotide sequence encoding the anti-CD200R1 antibody or the antigen binding fragment thereof disclosed herein or the bispecific antibody or the antigen binding fragment thereof disclosed herein.

Accordingly, the deoxyribonucleic acid (DNA) comprising a deoxynucleotide sequence encoding the anti-CD200R1 antibody or the antigen binding fragment thereof disclosed herein or the bispecific antibody or the antigen binding fragment thereof disclosed herein is used for treating a disease. In some embodiments, the disease is a cancer. In some embodiments, the cancer is selected from the group consisting of pancreatic ductal adenocarcinoma, prostate cancer, melanoma, liver cancer, breast cancer, lung cancer (e.g., lung squamous cell carcinoma), squamous cell carcinoma, ovarian cancer, leukemia, and myeloma.

In some embodiments, the deoxyribonucleic acid (DNA) may be introduced into the cells of a human body in vivo. In some embodiments, the deoxyribonucleic acid (DNA) of the invention is comprised in a vector or a delivering agent. In some embodiments, the deoxyribonucleic acid (DNA) of the invention is integrated into the genome of a cell. Accordingly, the ribonucleic acid (RNA) comprising a deoxynucleotide sequence encoding the anti-CD200R1 antibody or the antigen binding fragment thereof disclosed herein or the bispecific antibody or the antigen binding fragment thereof disclosed herein may be used for treating a disease. In some embodiments, the disease is a cancer. In some embodiments, the cancer is selected from the group consisting of pancreatic ductal adenocarcinoma, prostate cancer, melanoma, liver cancer, breast cancer, lung cancer (e.g., lung squamous cell carcinoma), squamous cell carcinoma, ovarian cancer, leukemia, and myeloma.

In some embodiments, the deoxyribonucleic acid (DNA) may be introduced into the cells of a human body in vivo. In some embodiments, the deoxyribonucleic acid (DNA) of the invention is comprised in a vector or a delivering agent.

### Vectors

In the fourth aspect, the invention further provides a vector, which comprises the nucleic acid comprising a nucleotide sequence encoding the anti-CD200R1 antibody or the antigen binding fragment thereof disclosed herein or the bispecific antibody or the antigen binding fragment thereof disclosed herein.

In some embodiments, the vector is a recombinant expression vector capable of expressing a polypeptide comprising a heavy or light chain variable region of an anti-CD200R1 antibody. For example, the invention provides recombinant expression vectors comprising any of the nucleic acid molecules mentioned above.

Any vector may be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pGAR, pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

A recombinant expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif). Bacteriophage vectors, such as λGT10, λGT11, λZapII (Stratagene), λEMBL4, and λNM1149, also may be used. Examples of plant expression vectors useful in the context of the disclosure comprise pBI01, pBI101.2, pBI101.3, pBIl21 and pBIN19 (Clontech). Examples of animal expression vectors useful in the context of the disclosure comprise pcDNA, pEUK-Cl, pMAM, and pMAMneo (Clontech).

Recombinant expression vectors may be prepared using standard recombinant DNA techniques described in, for example, Sambrook et al., Molecular Cloning: A Laboratory Manual, 3rd ed., Cold Spring Harbor Press, Cold Spring Harbor, N.Y 2001; and Ausubel et al., Current Protocols in Molecular Biology, Greene Publishing Associates and John Wiley & Sons, NY, 1994. Constructs of expression vectors, which are circular or linear, may be prepared to contain a replication system functional in a prokaryotic or eukaryotic host cell. Replication systems may be derived, e.g., from ColEl, 2μ plasmid, X, SV40, bovine papilloma virus, and the like.

Accordingly, the vector may be used for treating a disease. In some embodiments, the disease is a cancer. In some embodiments, the cancer is selected from the group consisting of pancreatic ductal adenocarcinoma, prostate cancer, melanoma, liver cancer, breast cancer, lung cancer (e.g., lung squamous cell carcinoma), squamous cell carcinoma, ovarian cancer, leukemia, and myeloma.

The vector of the invention may be introduced into a cell. In some embodiments, the vector of the invention may be introduced into a cell in vitro or ex vivo. Optionally, the cell introduced with the vector may subsequently be administered into the body of a subject. In some embodiments, the vector of the invention may be introduced into a cell in vivo.

For example, the vector may be a adenoviral vector comprising a nucleotide sequence encoding the anti-CD200R1 antibody or the antigen binding fragment thereof disclosed herein or the bispecific antibody or the antigen binding fragment thereof disclosed herein. The vector may be administered into the body of a subject, and then enter into a cell of the subject in vivo, thereby the nucleotide sequence encoding the anti-CD200R1 antibody or the antigen binding fragment thereof disclosed herein or the bispecific antibody or the antigen binding fragment thereof disclosed herein is integrated into the genome of the cell, and subsequently the cell expresses the anti-CD200R1 antibody or the antigen binding fragment thereof disclosed herein so as to treat the diseases disclosed herein.

### Host Cells

In the fifth aspect, the invention further provides a host cell comprising the nucleic acid disclosed herein or the vector disclosed herein.

Any cell may be used as a host cell for the nucleic acids or the vectors of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. lichenformis; Pseudomonas such as P. aeruginosa; and Streptomyces. In some embodiments, the cell is a human cell.

In some embodiments, the cell is an immune cell. In some embodiments, host cells include, for example, CHO cells, such as CHOS cells and CHOK1 cells, or HEK293 cells, such as HEK293A, HEK293T and HEK293FS.

The host cell of the invention is prepared by introducing the vector disclosed herein or the nucleic acid disclosed herein in vitro or ex vivo. The host cell of the invention may be administered into the body of a subject, and the host cell expresses the anti-CD200R1 antibody or the antigen binding fragment thereof disclosed herein in vivo so as to treat the diseases disclosed herein.

### Antibody-Drug Conjugate

In the sixth aspect, the invention provides an antibody-drug conjugate (ADC), comprising the antibody or the antigen-binding fragment thereof of the first aspect of the invention or the bi-specific antibody of the second aspect of the invention.

In the context of the present disclosure, a “conjugate” is an antibody or antibody fragment (such as an antigen-binding fragment) covalently linked to an effector molecule or a second protein (such as a second antibody). The effector molecule can be, for example, a drug, toxin, therapeutic agent, detectable label, protein, nucleic acid, lipid, nanoparticle, carbohydrate or recombinant virus. An antibody conjugate is often referred to as an “immunoconjugate.” When the conjugate comprises an antibody linked to a drug (e.g., a cytotoxic agent), the conjugate is often referred to as an “antibody-drug conjugate” or “ADC.” Other antibody conjugates include, for example, multi-specific (such as bispecific or trispecific) antibodies.

In some embodiments, the effector molecule can be a detectable label or an immunotoxin. Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain 1a of PE and the B chain of DT) and replacing it with a different targeting moiety, such as an antibody. The term “conjugated” or “linked” may refer to making two polypeptides into one contiguous polypeptide molecule. In one embodiment, an antibody is joined to an effector molecule. In another embodiment, an antibody joined to an effector molecule is further joined to a lipid or other molecule to a protein or peptide to increase its half-life in the body. The linkage can be either by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule.

The invention provides immunoconjugates that include a monoclonal antibody or antigen-binding fragment disclosed herein and an effector molecule. In some embodiments, the effector molecule is a toxin, such as, but not limited to, Pseudomonas exotoxin or a variant thereof. In other embodiments, the effector molecule is a detectable label, such as, but not limited to, a fluorophore, an enzyme or a radioisotope.

The disclosed monoclonal antibodies can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as a tumor cell). Conversely, where it is desired to invoke a non-lethal biological response, the therapeutic agent can be conjugated to a non-lethal pharmacological agent or a liposome containing a non-lethal pharmacological agent.

With the therapeutic agents and antibodies described herein, one of skill can readily construct a variety of clones containing functionally equivalent nucleic acids, such as nucleic acids which differ in sequence but which encode the same effector moiety or antibody sequence. Thus, the present disclosure provides nucleic acids encoding antibodies and conjugates and fusion proteins thereof.

Effector molecules can be linked to an antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site.

Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies one skilled in the art will be able to determine a suitable method for attaching a given agent to an antibody or other polypeptide.

The antibodies disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a pharmaceutical agent, and/or a protein or peptide that can mediate association of the antibody or antibody portion with another molecule (such as a strep tavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

The antibody can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP) and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

An antibody may be fused to a self-labelling protein tag (e.g. HaloTag). For example, the protein tag could be cloned at the end of a constant region. HaloTag is a self-labelling protein tag derived from a bacterial enzyme (a haloalkane dehalogenase), designed to covalently bind to a synthetic ligand. In some instances, the synthetic ligand comprises a chloroalkane linker attached to a fluorophore, such as a near-infrared fluorophore (Los et al. (2008) ACS Chem Biol. 3(6):373-82).

An antibody may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitope recognized by a secondary reporter (such as leucine zipper pair sequences, binding sites for secondary antibodies, metal binding domains, epitope tags). In some embodiments, labels are attached by spacer arms of various lengths to reduce potential steric hindrance.

An antibody can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I. An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one embodiment, the toxin is Pseudomonas exotoxin (PE) (U.S. Pat. No. 5,602,095). As used herein “Pseudomonas exotoxin” refers to a full-length native (naturally occurring) PE or a PE that has been modified. Such modifications can include, but are not limited to, elimination of domain 1a, various amino acid deletions in domains 1b, II and III, single amino acid substitutions and the addition of one or more sequences at the carboxyl terminus (for example, see Siegall et al. Biol. Chem. 264:14256-14261, 1989).

PE employed with the monoclonal antibodies described herein can include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell. Cytotoxic fragments of PE include PE40, PE38, and PE35. For additional description of PE and variants thereof, see for example, U.S. Pat. Nos. 4,892,827; 5,512,658; 5,602,095; 5,608,039; 5,821,238; and 5,854,044; U.S. Patent Application Publication No. 2015/0099707; PCT Publication Nos. WO 99/51643 and WO 2014/052064; Pai et al., Proc. Natl. Acad. Sci. USA 88:3358-3362, 1991; Kondo et al., J. Biol. Chem. 263:9470-9475, 1988; Pastan et al., Biochim. Biophys. Acta 1333:C1-C6, 1997.

Also contemplated herein are protease-resistant PE variants and PE variants with reduced immunogenicity, such as, but not limited to PE-LR, PE-6X, PE-8X, PE-LR/6X and PE-LR/8X (see, for example, Weldon et al, Blood 113(16):3792-3800, 2009; Onda et al, Proc Natl Acad Sci USA 105(32): 11311-11316, 2008; and PCT Publication Nos. WO 2007/016150, WO 2009/032954 and WO 2011/032022, which are herein incorporated by reference).

In some examples, the PE is a variant that is resistant to lysosomal degradation, such as PE-LR (Weldon et al, Blood 113(16):3792-3800, 2009; PCT Publication No. WO 2009/032954). In other examples, the PE is a variant designated PE-LR/6X (PCT Publication No. WO 2011/032022). In other examples, the PE variant is PE with reducing immunogenicity. In yet other examples, the PE is a variant designated PE-LR/8M (PCT Publication No. WO 2011/032022).

Modification of PE may occur in any previously described variant, including cytotoxic fragments of PE (for example, PE38, PE-LR and PE-LR/8M). Modified PEs may include any substitution(s), such as for one or more amino acid residues within one or more T-cell epitopes and/or B cell epitopes of PE, or deletion of one or more T-cell and/or B-cell epitopes (see, for example, U.S. Patent Application Publication No. 2015/0099707). Contemplated forms of PE also include deimmunized forms of PE, for example versions with domain II deleted (for example, PE24). Deimmunized forms of PE are described in, for example, PCT Publication Nos. WO 2005/052006, WO 2007/016150, WO 2007/014743, WO 2007/031741, WO 2009/32954, WO 2011/32022, WO 2012/154530, and WO 2012/170617.

The antibodies described herein can also be used to target any number of different diagnostic or therapeutic compounds to cells expressing the tumor or viral antigen on their surface. Thus, an antibody of the present disclosure can be attached directly or via a linker to a drug that is to be delivered directly to cells expressing cell-surface antigen. This can be done for therapeutic, diagnostic or research purposes. Therapeutic agents include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to an antibody can be an encapsulation system, such as a nanoparticle, liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well known to those of skill in the art (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

ADCs are compounds comprised of a tumor antigen-specific antibody (or antigen-binding fragment thereof) and a drug, typically a cytotoxic agent, such as an anti-microtubule agent or cross-linking agent. Because ADCs are capable of specifically targeting cancer cells, the drug can be much more potent than agents used for standard chemotherapy. The most common cytotoxic drugs currently used with ADCs have an IC50 that is 100- to 1000-fold more potent than conventional chemotherapeutic agents. Common cytotoxic drugs include anti-microtubule agents, such as maytansinoids and auristatins (such as auristatin E and auristatin F). Other cytotoxins for use with ADCs include pyrrolobenzodiazepines (PDBs), which covalently bind the minor groove of DNA to form interstrand crosslinks. In many instances, ADCs comprise a 1:2 to 1:4 ratio of antibody to drug (Bander, Clinical Advances in Hematology & Oncology 10(8; suppl 10):3-7, 2012).

The antibody and drug can be linked by a cleavable or non-cleavable linker. However, in some instances, it is desirable to have a linker that is stable in the circulation to prevent systemic release of the cytotoxic drug that could result in significant off-target toxicity. Non-cleavable linkers prevent release of the cytotoxic agent before the ADC is internalized by the target cell. Once in the lysosome, digestion of the antibody by lysosomal proteases results in the release of the cytotoxic agent (Bander, Clinical Advances in Hematology & Oncology 10(8; suppl 10):3-7, 2012).

One method for site-specific and stable conjugation of a drug to a monoclonal antibody is via glycan engineering. Monoclonal antibodies have one conserved N-linked oligosaccharide chain at the Asn297 residue in the CH2 domain of each heavy chain (Qasba et al., Biotechnol Prog 24:520-526, 2008). Using a mutant 1,4-galactosyltransferase enzyme (Y289L-Gal-Tl; U.S. Patent Application Publication Nos. 2007/0258986 and 2006/0084162, herein incorporated by reference), 2-keto-galactose is transferred to free GlcNAc residues on the antibody heavy chain to provide a chemical handle for conjugation.

The oligosaccharide chain attached to monoclonal antibodies can be classified into three groups based on the terminal galactose residues—fully galactosylated (two galactose residues; IgG-G2), one galactose residue (IgG-Gl) or completely degalactosylated (IgG-GO). Treatment of a monoclonal antibody with 1,4-galactosidase converts the antibody to the IgG-GO glycoform. The mutant 1,4-galactosyltransferase enzyme is capable of transferring 2-keto-galactose or 2-azido-galactose from their respective UDP derivatives to the GlcNAc residues on the IgG-Gl and IgG-GO glycoforms. The chemical handle on the transferred sugar enables conjugation of a variety of molecules to the monoclonal antibody via the glycan residues (Qasba et al., Biotechnol Prog 24:520-526, 2008).

Provided herein are ADCs that include a drug (such as a cytotoxic agent) conjugated to a monoclonal antibody that binds (such as specifically binds) CD200R1. In some embodiments, the drug is a small molecule. In some examples, the drug is a cross-linking agent, an anti-microtubule agent and/or anti-mitotic agent, or any cytotoxic agent suitable for mediating killing of tumor cells.

Exemplary cytotoxic agents include, but are not limited to, a PDB, an auristatin, a maytansinoid, dolastatin, calicheamicin, nemorubicin and its derivatives, PNU-159682, anthracycline, vinca alkaloid, taxane, trichothecene, CC1065, camptothecin, elinafide, a combretastain, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity.

In some embodiments, the ADC comprises a pyrrolobenzodiazepine (PBD). The natural product anthramycin (a PBD) was first reported in 1965 (Leimgruber et al, J Am Chem Soc, 87:5793-5795, 1965; Leimgruber et al., JAm Chem Soc, 87:5791-5793, 1965). Since then, a number of PBDs, both naturally-occurring and synthetic analogues, have been reported (Gerratana, Med Res Rev 32(2):254-293, 2012; and U.S. Pat. Nos. 6,884,799; 7,049,311; 7,067,511; 7,265,105; 7,511,032; 7,528,126; and 7,557,099). As one example, PDB dimers recognize and bind to specific DNA sequences, and have been shown to be useful as cytotoxic agents. PBD dimers have been conjugated to antibodies and the resulting ADC shown to have anti-cancer properties (see, for example, US 2010/0203007). Exemplary linkage sites on the PBD dimer include the five-membered pyrrolo ring, the tether between the PBD units, and the N10-C11 imine group (see WO 2009/016516; US 2009/304710; US 2010/047257; US 2009/036431; US 2011/0256157; and WO 2011/130598).

In some embodiments, the ADC comprises an antibody conjugated to one or more maytansinoid molecules. Maytansinoids are derivatives of maytansine, and are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

In some embodiments, the ADC includes an antibody conjugated to a dolastatin or auristatin, or an analog or derivative thereof (see U.S. Pat. Nos. 5,635,483; 5,780,588; 5,767,237; and 6, 124,431). Auristatins are derivatives of the marine mollusk compound dolastatin-10. Dolastatins and auristatins have been shown to interfere with microtubule dynamics, GTP hydrolysis, and nuclear and cellular division (Woyke et al., Antimicrob Agents and Chemother 45(12):3580-3584, 2001) and have anticancer (U.S. Pat. No. 5,663,149) and antifungal activity (Pettit et al., Antimicrob Agents Chemother 42:2961-2965, 1998). Exemplary dolastatins and auristatins include, but are not limited to, dolastatin 10, auristatin E, auristatin F, auristatin EB (AEB), auristatin EFP (AEFP), MM AD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), and other auristatins (see, for example, U.S. Publication No. 2013/0129753).

In some embodiments, the ADC comprises an antibody conjugated to one or more calicheamicin molecules. The calicheamicin family of antibiotics, and analogues thereof, are capable of producing double-stranded DNA breaks at sub-picomolar concentrations (Hinman et al, Cancer Res 53:3336-3342, 1993; Lode et al, Cancer Res 58:2925-2928, 1998). Exemplary methods for preparing ADCs with a calicheamicin drug moiety are described in U.S. Pat. Nos. 5,712,374; 5,714,586; 5,739,116; and 5,767,285.

In some embodiments, the ADC comprises an anthracycline. Anthracyclines are antibiotic compounds that exhibit cytotoxic activity. It is believed that anthracyclines can operate to kill cells by a number of different mechanisms, including intercalation of the drug molecules into the DNA of the cell thereby inhibiting DNA-dependent nucleic acid synthesis; inducing production of free radicals which then react with cellular macromolecules to cause damage to the cells; and/or interactions of the drug molecules with the cell membrane. Non-limiting exemplary anthracyclines include doxorubicin, epirubicin, idarubicin, daunomycin, daunorubicin, doxorubicin, epirubicin, nemorubicin, valrubicin and mitoxantrone, and derivatives thereof. For example, PNU-159682 is a potent metabolite (or derivative) of nemorubicin (Quintieri et al, Clin Cancer Res 11(4): 1608-1617, 2005). Nemorubicin is a semisynthetic analog of doxorubicin with a 2-methoxymorpholino group on the glycoside amino of doxorubicin (Grandi et al, Cancer Treat Rev 17:133, 1990; Ripamonti et al, Br J Cancer 65:703-707, 1992).

In some embodiments, the ADC can further include a linker. In some examples, the linker is a bifunctional or multifunctional moiety that can be used to link one or more drug moieties to an antibody to form an ADC. In some embodiments, ADCs are prepared using a linker having reactive functionalities for covalently attaching to the drug and to the antibody. For example, a cysteine thiol of an antibody can form a bond with a reactive functional group of a linker or a drug-linker intermediate to make an ADC.

In some examples, a linker has a functionality that is capable of reacting with a free cysteine present on an antibody to form a covalent bond. Exemplary linkers with such reactive functionalities include maleimide, haloacetamides, oc-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some examples, a linker has a functionality that is capable of reacting with an electrophilic group present on an antibody. Examples of such electrophilic groups include, but are not limited to, aldehyde and ketone carbonyl groups. In some cases, a heteroatom of the reactive functionality of the linker can react with an electrophilic group on an antibody and form a covalent bond to an antibody unit. Non-limiting examples include hydrazide, oxime, amino, hydrazine, thiosemicarbazone, hydrazine carboxylate and arylhydrazide.

In some examples, the linker is a cleavable linker, which facilitates release of the drug. Examples of cleavable linkers include acid-labile linkers (for example, comprising hydrazone), protease-sensitive linkers (for example, peptidase-sensitive), photolabile linkers, and disulfide-containing linkers (Chari et al, Cancer Res 52:127-131, 1992; U.S. Pat. No. 5,208,020).

The ADCs disclosed herein can be used for the treatment of a cancer alone or in combination with another therapeutic agent and/or in combination with any standard therapy for the treatment of cancer (such as surgical resection of the tumor, chemotherapy or radiation therapy), wherein the microenvironment of the cancer comprises T cells and/or myeloid cells on the surface of which CD200R1 is highly expressed or the cancer is responsive to decreasing, inhibiting and/or blocking immune regulatory function or activity mediated by CD200R1.

### Pharmaceutical Compositions

In the seventh aspect, the invention provides a pharmaceutical composition comprising (i) the antibody or the antigen binding fragment thereof of the first aspect of the invention, or the bi-specific antibody of the second aspect of the invention, or the nucleic acid of the third aspect of the invention, or the vector of the fourth aspect of the invention, or the host cell of the fifth aspect of the invention, or the ADC of the sixth aspect of the invention; and optionally (ii) a pharmaceutically acceptable carrier or excipient.

The invention provides pharmaceutical composition comprising an antibody of the invention. In some embodiments, the pharmaceutical composition further comprises a pharmaceutically acceptable carrier. The term “pharmaceutically acceptable carrier” includes any and all solvents, buffers, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e. g. by injection or infusion). For example, in some embodiments, a composition for intravenous administration typically is a solution in sterile isotonic aqueous buffer.

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention provides therapeutic compositions comprising the anti-CD200R1 antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

### Therapeutic Methods

In the eighth aspect, the invention provides a method of treating a cancer in a subject, comprising administering to the subject an effective amount of the antibody or the antigen binding fragment thereof, the bi-specific antibody, the nucleic acid, the vector, the host cell, the ADC, or the pharmaceutical composition of the invention.

In a ninth aspect, the invention provides an effective amount of the antibody or the antigen binding fragment thereof, the bi-specific antibody, the nucleic acid, the vector, the host cell, the ADC, or the pharmaceutical composition of the invention for use in a method of treating a cancer in a subject.

In a tenth aspect, the invention provides use of the antibody or the antigen binding fragment thereof, the bi-specific antibody, the nucleic acid, the vector, the host cell, the ADC, or the pharmaceutical composition of the invention in the manufacture of a medicament for treating a cancer in a subject.

The antibodies provide herein can be administered to slow or inhibit the progression of a cancer, or inhibit the metastasis of a cancer. In these applications, a therapeutically effective amount of a composition is administered to a subject in an amount sufficient to inhibit growth, replication or metastasis of cancer cells, or to inhibit a sign or a symptom of the cancer. Suitable subjects may include those diagnosed with a cancer, wherein the microenvironment of the cancer comprises T cells and/or myeloid cells on the surface of which CD200R1 is highly expressed, such as mesothelioma, prostate cancer, lung cancer, stomach cancer, squamous cell carcinoma, pancreatic cancer, cholangiocarcinoma, triple negative breast cancer or ovarian cancer.

Provided herein is a method of treating a cancer in a subject by administering to the subject a therapeutically effective amount of an antibody described herein. Also provided herein is a method of inhibiting metastasis of a cancer in a subject by administering to the subject a therapeutically effective amount of an antibody described herein. In some embodiments, the cancer is pancreatic ductal adenocarcinoma, prostate cancer, melanoma, liver cancer, breast cancer, lung cancer (e.g., lung squamous cell carcinoma), squamous cell carcinoma, ovarian cancer, leukemia, or myeloma.

Administration of an antibody disclosed herein can also be accompanied by administration of other anti-cancer agents or therapeutic treatments (such as surgical resection of a tumor). Any suitable anti-cancer agent can be administered in combination with the antibodies disclosed herein. Exemplary anti-cancer agents include, but are not limited to, chemotherapeutic agents, such as, for example, mitotic inhibitors, alkylating agents, antimetabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and other antibodies that specifically target cancer cells.

Another common treatment for some types of cancer is surgical treatment, for example surgical resection of a metastatic tumor. Another example of a treatment is radiotherapy, for example administration of radioactive material or energy (such as external beam therapy) to the tumor site to help eradicate the tumor or shrink it prior to surgical resection.

### Methods for Diagnosis and Detection

In an eleventh aspect, the invention provides a method for determining a subject suffering from a cancer or having a risk of developing a cancer, wherein the microenvironment of the cancer comprises T cells and/or myeloid cells on the surface of which CD200R1 is highly expressed, wherein the method comprises:
(a) obtaining a biological sample from the subject,
(b) contacting the sample with the antibody or the antigen binding fragment thereof of the first aspect of the invention, and
(c) detecting binding of the antibody to the sample,
   wherein an increase in binding of the antibody or antigen binding fragment thereof to the sample as compared to binding of the antibody or antigen binging fragment thereof to a control sample indicates that the subject is suffering from a cancer or has a risk of developing a cancer.

In a twelfth aspect, the invention provides a method for imaging a cancer in a subject, wherein the microenvironment of the cancer comprises T cells and/or myeloid cells on the surface of which CD200R1 is highly expressed, wherein the method comprises:
(a) administering the antibody or antigen binding fragment thereof of the first aspect of the invention, wherein the antibody is conjugated to a detectable marker, and
(b) detecting the presence of the marker.

Methods are provided herein for detecting CD200R1 protein in vitro or in vivo. In some cases, CD200R1 expression is detected in a biological sample. The sample can be any sample, including, but not limited to, blood samples, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. A biological sample is typically obtained from a mammal, such as a human or non-human primate.

Provided herein is a method of determining if a subject has a cancer by contacting a sample from the subject with a CD200R1-specific monoclonal antibody disclosed herein; and detecting binding of the antibody to the sample, wherein the microenvironment of the cancer comprises T cells and/or myeloid cells on the surface of which CD200R1 is highly expressed. An increase in binding of the antibody to the sample as compared to binding of the antibody to a control sample identifies the subject as having a cancer, wherein the microenvironment of the cancer comprises T cells and/or myeloid cells on the surface of which CD200R1 is highly expressed.

In another embodiment, provided is a method of confirming a diagnosis of a cancer in a subject by contacting a sample from a subject diagnosed with a cancer with a CD200R1-specific monoclonal antibody disclosed herein; and detecting binding of the antibody to the sample, wherein the microenvironment of the cancer comprises T cells and/or myeloid cells on the surface of which CD200R1 is highly expressed. An increase in binding of the antibody to the sample as compared to binding of the antibody to a control sample confirms the diagnosis of a cancer in the subject, wherein the microenvironment of the cancer comprises T cells and/or myeloid cells on the surface of which CD200R1 is highly expressed.

In some examples of the disclosed methods, the monoclonal antibody is directly labeled. In other examples, the methods further include contacting a second antibody that specifically binds the monoclonal antibody with the sample; and detecting the binding of the second antibody. An increase in binding of the second antibody to the sample as compared to binding of the second antibody to a control sample detects a cancer in the subject or confirms the diagnosis of a cancer in the subject, wherein the microenvironment of the cancer comprises T cells and/or myeloid cells on the surface of which CD200R1 is highly expressed.

In some cases, the cancer is mesothelioma, prostate cancer, lung cancer, stomach cancer, squamous cell carcinoma, pancreatic cancer, cholangiocarcinoma, triple negative breast cancer or ovarian cancer.

In some examples, the control sample is a sample from a subject without cancer. In particular examples, the sample is a blood or tissue sample.

In some embodiments of the methods of diagnosis and detection, the anti-CD200R1 antibody is directly labeled with a detectable label. In another embodiment, the anti-CD200R1 antibody (the first antibody) is unlabeled and a second antibody or other molecule that can bind the first is labeled. As is well known to one of skill in the art, a secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative embodiment, CD200R1 can be assayed in a biological sample by a competition immunoassay utilizing CD200R1 protein standards labeled with a detectable substance and an unlabeled anti-CD200R1 antibody. In this assay, the biological sample, the labeled CD200R1 protein standards and the anti-CD200R1 antibody are combined and the amount of labeled CD200R1 protein standard bound to the unlabeled antibody is determined. The amount of CD200R1 in the biological sample is inversely proportional to the amount of labeled CD200R1 protein standard bound to the anti-CD200R1 antibody. The immunoassays and methods disclosed herein can be used for a number of purposes. In one embodiment, the anti-CD200R1 antibody may be used to detect the production of CD200R1 in cells in cell culture. In another embodiment, the antibody can be used to detect the amount of CD200R1 in a biological sample, such as a tissue sample, or a blood or serum sample. In some examples, the CD200R1 is cell-surface CD200R1. In other examples, the CD200R1 protein is soluble (e.g. in a cell culture supernatant or in a body fluid sample, such as a blood or serum sample).

In one embodiment, a kit is provided for detecting CD200R1 in a biological sample, such as a blood sample or tissue sample. For example, to confirm a cancer diagnosis in a subject, a biopsy can be performed to obtain a tissue sample for histological examination. Kits for detecting a polypeptide will typically comprise a monoclonal anti-CD200R1 antibody, such as any of the monoclonal antibodies disclosed herein. In a further embodiment, the antibody is labeled (for example, with a fluorescent, radioactive, or an enzymatic label). In one embodiment, a kit includes instructional materials disclosing means of use of an anti-CD200R1 antibody. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one embodiment, the diagnostic kit comprises an immunoassay. Although the details of the immunoassays may vary with the particular format employed, the method of detecting CD200R1 in a biological sample generally includes the steps of contacting the biological sample with an anti-CD200R1 antibody. The antibody is allowed to specifically bind under immunologically reactive conditions to form an immune complex, and the presence of the immune complex (bound antibody) is detected directly or indirectly.

The antibodies disclosed herein can also be utilized in immunoassays, such as, but not limited to radioimmunoassays (RIAs), ELISA, or immunohistochemical assays. The antibodies can also be used for fluorescence activated cell sorting (FACS). FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies that bind CD200R1, as disclosed herein, can be used in these assays. Thus, the antibodies can be used in a conventional immunoassay, including, without limitation, an ELISA, an RIA, FACS, tissue immunohistochemistry, Western blot or immunoprecipitation.

### EXAMPLES

The present invention is further illustrated by the following examples, which are not intended to limit the present invention. Experimental procedures without specified conditions in the following examples are performed in accordance with conventional procedures and conditions, or in accordance with instructions.

### Example 1. Assessment of the Expression of CD200 and CD200R1 on Tumor-Infiltrating Lymphocytes (TILs)

CD200 and CD200R1 have been shown to be upregulated in the tumor microenvironment of various cancers, which may contribute to the immune suppressive functions (Pathobiology 2021; 88:218-227; Cancers (Basel). 2021 March; 13(5): 1024). The expression of CD200R1 and PD1 on tumor-infiltrating lymphocytes (TTLs) from renal cell carcinoma (RCC) patients and on PBMCs from healthy donors was analyzed. Briefly, human blood samples from healthy donors were purchased from Research Blood Components (Watertown, MA). PBMCs were isolated by using Lymphoprep (STEMCELL). Fresh tumor tissue from cancer patients were provided by MT Group biospecimens (Los Angeles, CA). Tumor tissue was dissociated, digested in 500 g/ml DNase (Roche), 1 mg/ml Dispase (Gibco Life Tech) and 1 mg/ml Collagenase P (Roche). Single cell suspension was collected, washed, and used for analysis. The expressions of CD200, CD200R1 and PD1 were analyzed by FACS staining. PBMCs or tumor single cell suspension were stained with CD200 antibody (BioLegend), CD200R1 antibody (BioLegend) and PD1 antibody (BioLegend), and were analyzed. CD45 positive cell population in tumor single cell suspension was identified as TTLs.

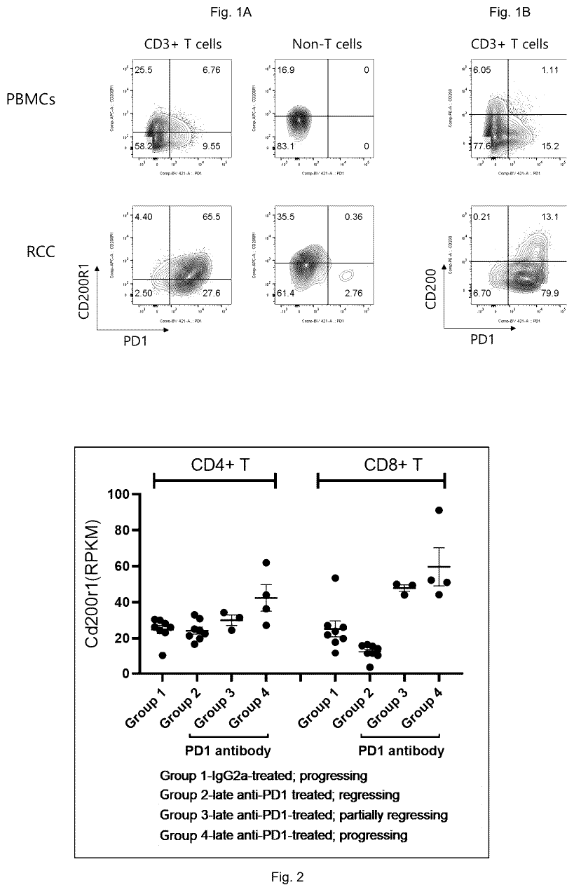

For PBMCs from healthy donors, CD200R1 expression level on T cells and non-T cells from healthy PBMCs are low. For tumor-infiltrating lymphocytes (TTLs) from renal cell carcinoma patients, CD200R1 expression was highly upregulated on T cells from tumor microenvironment, especially on PD1+ T cells (FIG. 1A); in the non-T cell population, CD200R1 was also upregulated (FIG. 1A); in addition, CD200 expression was upregulated on T cells especially on PD1+ T cells as well. (FIG. 1B).

### Example 2. Analysis of CD200R1 Expression in PD1-Treated Mice

A HKP1 (KrasG12Dp53−/−) orthotopic, immunocompetent, syngeneic preclinical model of non-small cell lung cancer (NSCLC) has been used to explore the therapeutic efficacy of PD-1/PD-L1 inhibition. CD4 and CD8 lung tumor infiltrating lymphocytes were obtained from IgG antibody (IgG2a, as a control) or anti-PD1 antibody treated C57Bl/6 mice at day 14, 17 or 24, and were sorted into RLT lysis buffer for mRNA-Sequencing (as shown in JCI Insight. 2018 Jul. 12; 3(13): e96836.). mRNA sequencing data were downloaded from NCBI (GSE114300).

To explore the association of CD200R1 expression with the therapeutic effect of anti-PD1 treatment, expression of the gene cd200r1 in the IgG2a treated group with progressing tumor growth, the anti-PD1 treated group with regressing tumor, the anti-PD1 treated group with partial regressing, and the anti-PD1 treated group with progressing was analyzed. The results show that the expression of the gene cd200r1 (mRNA) was upregulated on CD4+ and CD8+ T cells in PD1-progressing groups (FIG. 2).

### Example 3. Generation of Anti-CD200R1 Antibody Molecules

### 3.1 Immunization of Mice

The Harbour H2L2 mice (obtained from Harbour Antibodies BV) are transgenic mice that carry human immunoglobulin immune repertoire, and the antibody generated by the transgenic mice has fully human sequences. The Harbour H2L2 mice were immunized in multiple rounds with soluble recombinant human CD200R1 isoform a (CD200R1a)-Fc fusion protein (SEQ ID NO: 178) as antigen. The antigenic protein was mixed with an immunoadjuvant to form an immunogenic reagent, which was then injected subcutaneously via the groin or intraperitoneally.

In each round of immunization, each mouse received a total injection dose of 100 μL. In the first round of immunization, each mouse received the immunization with an immunogenic reagent prepared by mixing 50 g of the antigenic protein with complete Freund's adjuvant (Sigma, #F5881) in a 1:1 volume ratio. In each subsequent round of booster immunization, each mouse received an immunization with an immunogenic reagent prepared by mixing 25 g of the antigenic protein with Sigma Adjuvant System adjuvant (Sigma, #S6322). In general, there were 6-7 rounds of booster immunizations. The immunization was performed at Days 0, 14, 28, 42, 56, 70, 84 and 98; and the antibody titers in serum of mice were measured at Days 49 and 77. Five days after the last round of booster immunization, splenic B cells were isolated from the Harbour H2L2 mouse.

### 3.2 Serum Titer Assay

At the above specific time points (at Days 49 and 77), the serum of mice was collected, and the titer of antibody binding to CD200R1 in the serum was determined by the FACS method.

The cDNA encoding human CD200R1 isoform a (CD200R1a) (NP_620161.1, SEQ ID NO: 172) or isoform d (NP_740750.1, SEQ ID NO: 173) was obtained by gene synthesis and cloned into a lentiviral vector pGWLV11 (AZENTA Life sciences). The lentiviral particles were generated, then transfected CHOK1 cells to generate CHOK1/hCD200R1a and CHOK1/hCD200R1d cells that highly express human CD200R1 isoform a (CD200R1a) or isoform d respectively (AZENTA Life sciences).

The serially diluted mouse serum was incubated with CHOK1-hCD200R1a cells at 4° C. for 1 h; after the cells were washed twice, a secondary anti-rat IgG (H+L) (Life technologies, A11006) was added and incubated at 4° C. for 1 h, and then the resulting cells were washed twice, resuspended, and detected by a flow cytometer (BD, CantoII). CHOK1 cells served as background controls. Immunized mice having high serum titer of anti-CD200R1 were selected.

### 3.3 Screening of Anti-CD200R1 Antibodies by Hybridoma Technology

The cDNA encoding Cynomolgus macaques CD200R1a (NP_001305106.1, SEQ ID NO:175) was obtained by gene synthesis and cloned into a lentiviral vector pGWLV11(AZENTA Life sciences). The lentiviral particles were generated, then transfected CHOK1 cells to generate CHOK1/cynoCD200R1a cells that highly expressed Cynomolgus macaques CD200R1 isoform a (AZENTA Life sciences).

The selected immunized mice having high serum titer of anti-CD200R1 were applied for the last round of immunization and then sacrificed. Splenocytes and SP2/0 myeloma cells (ATCC, CRL-1581) were electrofused at a cell ratio of 4:1 with the electrofusion parameters shown as follows: V1: 50V, t1: 15 s, V2: 600 V, t2: 20 ρs, t3: 0.5 s, n: 1, t4: 7 s, V+/−: +, and fade: on. The cells were resuspended in DMEM medium containing 20% FBS and HT, and plated in 1×10⁵ cells/100 μL/well. After 24 h, DMEM containing 20% FBS and 2×HT was added in 100 μL/well for further culturing. The supernatant was subsequently collected and detected for the antibody titer. In general, 9-15 days after the fusion, supernatant of hybridoma derived from Splenocytes of mice immunized with the recombinant human CD200R1-his protein is collected and primarily screened by ELISA, and detected for the binding to the recombinant human CD200R1-his protein (Sino Biological, Cat: 11218-H08H). The positive clones are then further confirmed by FACS, and detected for the binding ability to CHOK1/hCD200R1a and CHOK1/cynoCD200R1a. In addition, the blocking effect of positive clones on the binding of human CD200 to CHOK1/hCD200R1a was detected by the FACS method. Positive wells with blocking effect were further subcloned by the limiting dilution method, and further screened by ELISA and FACS. Clones with significant binding to human and Cyno CD200R1a were selected for sequencing.

In this example, the sequences of the variable domains of the anti-CD200R1 monoclonal antibody molecules obtained from immunized Harbour H2L2 mice were human antibody sequences.

### 3.4 Preparation and Purification of Fully Human Recombinant IgG1 Antibodies

After obtaining the sequences encoding the light and heavy chain variable region of each screened antibody molecule, the nucleic acid sequences encoding the light and heavy chain variable region of each antibody molecule were fused with the nucleic acid sequences encoding the light and heavy chain constant domains of the human antibody and expressed by recombinant DNA techniques to obtain recombinant antibody molecules. Specifically, the nucleic acid sequence encoding the heavy chain variable region (VH) of the antibody was genetically synthesized and cloned into a mammalian cell expression plasmid vector (pTT5 mammalian expression vector) comprising the nucleic acid sequence encoding the heavy chain constant domain of the human IgG1 antibody so as to encode a full-length heavy chain. The nucleic acid sequence encoding the light chain variable domain (VL) of the antibody was genetically synthesized and cloned into a mammalian cell expression plasmid vector (pTT5 mammalian expression vector) comprising the nucleic acid sequence encoding the light chain constant region of the human Igκ antibody so as to encode a full-length light chain.

The plasmid encoding the heavy chain of the antibody and the plasmid encoding the light chain of the antibody were simultaneously transfected into human embryonic kidney cell HEK293, and a purified recombinant anti-CD200R1 antibody with light and heavy chain correctly assembled in pairs can be obtained by the conventional recombinant protein expression and purification techniques.

Specifically, HEK293 cells were expanded in FreeStyle™ F17 Expression Medium (Thermo, #A1383504). Before the transient transfection, the cells were adjusted to a concentration of 6-8×10⁵ cells/mL, and cultured in a shaker at 37° C. with 8% CO₂ for 24 h to a concentration of about 1.2×10⁶ cells/mL. 30 mL of cultured cells were taken. The plasmid comprising the nucleic acid sequence encoding the heavy chain of the antibody and the plasmid comprising the nucleic acid sequence encoding the light chain of the antibody described above were mixed in a ratio of 2:3, with a total of 30 g of plasmids dissolved in 1.5 mL of Opti-MEM reduced serum medium (Thermo, 31985088), and the medium was filtered through a 0.22 m filter for sterilization. Then, 1.5 mL of Opti-MEM was mixed with 120 μL of 1 mg/mL PEI (Polysciences, Inc. #23966-2), and left to stand for 5 min. PEI was slowly added to the plasmid, and incubated at room temperature for 10 min. The mixed solution of plasmid and PEI was slowly dropped into the culture flask while shaking, and cultured in a shaker at 37° C. with 8% CO₂ for 5 days. Cell viability was measured after 5 days. The culture was collected and centrifuged at 3300 g for 10 min, and then the supernatant was collected and centrifuged at high speed to remove impurities. A gravity column (Bio-Rad, #7311550) containing MabSelect™ (GE Healthcare Life Science, #71-5020-91 AE) was equilibrated with PBS (pH 7.4) and rinsed with 2-5 column volumes of PBS. The column was loaded with the supernatant sample, and rinsed with 5-10 column volumes of PBS, followed by 0.1 M glycine at pH 3.5 to elute the target protein. The eluate was adjusted to neutrality with Tris-HCl at pH 8.0, and concentrated and buffer exchanged into PBS buffer with an ultrafiltration tube (Millipore, UFC901024) to obtain a purified solution of anti-CD200R1 antibody. Finally, the purified solution was determined for concentration by using NanoDrop (Thermo Scientific™ NanoDrop™ One), and then subpackaged and stored for later use.

### 3.5 Sequences of Fully Human Recombinant Anti-CD200R1 Antibodies

After screening and sequencing, the amino acid sequences of the light and heavy chain variable regions, the light chain, the heavy chain and the CDRs defined according to the Chothia scheme of the CD200R1 antibodies in the present application are listed in Error! Reference source not found.4.

The reference antibody PR002906 used in the various examples of the present application was the corresponding recombinant IgG1 antibody of huDX182. The sequences of huDX182 were derived from U.S. Pat. No. 8,212,008B2. The heavy chain sequence and the light chain sequence of PR002906 are set forth in SEQ ID NO: 139 and SEQ ID NO: 156, respectively.

The reference antibody PR002903 used in the various examples of the present application was the corresponding recombinant antibody of hB7V3V2-hG2G4. The sequences of hB7V3V2-hG2G4 were derived from U.S. Pat. No. 8,075,884B2. The heavy chain sequence and the light chain sequence of PR002903 were set forth in SEQ ID NO: 138 and SEQ ID NO: 155, respectively.

The reference antibody PR006207 used in the various examples of the present application was the corresponding recombinant antibody of I-4P. The sequences of I-4P were derived from Patent Application No. WO2020055943A1. The heavy chain sequence and the light chain sequence of PR006207 were set forth in SEQ ID NO: 144 and SEQ ID NO: 160, respectively.

The reference antibody PR200691 used in the various examples of the present application were derived from Patent Application No. WO2021096753A1. The heavy chain sequence and the light chain sequence of PR200691 were set forth in SEQ ID NO: 181 and SEQ ID NO: 182, respectively.

The reference antibody PR200526 (namely IgG1) was used as a negative control, the amino acid sequence of the heavy chain is set forth in SEQ ID NO: 179, and the amino acid sequence of the light chain is set forth in SEQ ID NO: 180.

TABLE 4
numbering of anti-CD200R1 antibodies

| Antibody | Light | Heavy | | | | | | | | |
|---|---|---|---|---|---|---|---|---|---|---|
| No. | Chain | Chain | VL | VH | LCDR1 | LCDR2 | LCDR3 | HCDR1 | HCDR2 | HCDR3 |
| PR002903 | 155 | 138 | 121 | 106 | 60 | 74 | 89 | 8 | 23 | 42 |
| PR002906 | 156 | 139 | 122 | 107 | 61 | 75 | 90 | 9 | 24 | 43 |
| PR006207 | 160 | 144 | 126 | 112 | 63 | 77 | 94 | 12 | 27 | 47 |
| PR005474 | 157 | 140 | 123 | 108 | 62 | 76 | 91 | 10 | 25 | 44 |
| PR005509 | 158 | 141 | 124 | 109 | 62 | 76 | 92 | 11 | 26 | 45 |
| PR005512 | 159 | 142 | 125 | 110 | 62 | 76 | 93 | 11 | 25 | 45 |
| PR005514 | 158 | 143 | 124 | 111 | 62 | 76 | 92 | 11 | 26 | 46 |
| PR006475 | 161 | 145 | 127 | 113 | 64 | 78 | 95 | 13 | 28 | 48 |
| PR006480 | 162 | 146 | 128 | 114 | 65 | 79 | 96 | 11 | 29 | 49 |

### Example 4: Measurements of the Binding Affinity of the Anti-CD200R1 Antibodies to CD200R1a

To determine the binding affinities of the anti-CD200R1 antibodies to CD200R1a proteins of various species (mouse, human, Cynomolgus macaques), bio-layer interferometry (BLI) assays were carried out using an Octet® RED96e instrument. Antibodies were produced as described in Example 3.4 and diluted to 5 μg/mL using freshly prepared 1× kinetic buffer (10× kinetic buffer (Catalog #18-1105, ForteBio) was diluted with PBS(Catalog #E607016-0500, BBI Life Sciences)) and were captured on the surface of anti-human Fc (AHC) Octet biosensors (Catalog #18-5060, ForteBio) to reach capture levels between 0.9-1.2 nm. The biosensors having the captured anti-CD200R1 antibodies were then dipped in wells containing 2-fold serial dilutions of CD200R1a proteins to detect association signals, followed by dissociation steps in wells containing 1× kinetic buffer. The association phase and dissociation phase were shown in Table 5. The sensorgrams were recorded and the reference signals were subtracted before curve fitting using ForteBio Data Analysis 11.0 software. Association rates (kₒₙ) and dissociation rates (k_{off}) were calculated using a simple one-to-one Langmuir binding model. The equilibrium dissociation constant (K_{D}) was calculated as the ratio of k_{off}/kₒₙ. The binding affinity of human CD200R1a to human CD200 was determined with the same protocol, using Fc-tagged human CD200 protein instead of the anti-CD200R1 antibodies, and 2-fold serial dilutions of human CD200R1a protein.

All screened antibodies (PR005474, PR005509, PR005512, PR005514, PR006475, and PR006480) did not bind to mouse CD200R1 (data not shown). The data for binding affinity of anti-CD200R1 antibodies with human CD200R1a and Cynomolgus macaques CD200R1a are summarized below in Table 5. As shown in Table 5, antibody clones PR005474, PR005509, PR60-012, PR60814, PR006475, and PR006480 have higher binding affinity to human CD200R1a, as compared to hCD200 or the reference antibody PR002906. The antibodies PR005474, PR005509, PRE04512, and PR006480 have higher binding affinity to cyno CD200R1a, as compared to hCD200 or the reference antibody PR002906.

TABLE 5
Binding Affinity of the anti-CD200R1 antibodies to human or cyno CD200R1a

| | Loading | Conc. | | | | |
|---|---|---|---|---|---|---|
| Antigen | Sample ID | (nM) | KD (M) | kon(1/Ms) | kdis(1/s) | Full R\hat{ }2 |
| Human | hCD200 | 240-60 | 7.71E−08 | 2.30E+05 | 1.77E−02 | 0.9877 |
| CD200R1a | PR002906 | 160-40 | 3.93E−08 | 8.67E+04 | 3.41E−03 | 0.9956 |
| | PR005474 | 160-40 | 1.66E−08 | 6.07E+04 | 1.01E−03 | 0.9966 |
| | PR005509 | 160-40 | 7.74E−09 | 6.61E+04 | 5.11E−04 | 0.9948 |
| | PR005512 | 160-40 | 8.65E−09 | 7.51E+04 | 6.49E−04 | 0.9966 |
| | PR005514 | 160-40 | 5.83E−09 | 5.42E+04 | 3.16E−04 | 0.9988 |
| | PR006475 | 240-60 | 1.28E−08 | 2.94E+04 | 3.76E−04 | 0.9885 |
| | PR006480 | 160-40 | 1.43E−08 | 3.33E+04 | 4.76E−04 | 0.9951 |
| Cyno | hCD200 | 240-60 | 1.97E−07 | 1.61E+05 | 3.16E−02 | 0.9823 |
| CD200R1a | PR002906 | 160-40 | Not detectable | | | |
| | PR005474 | 160-40 | 1.47E−07 | 1.96E+05 | 2.87E−02 | 0.9803 |
| | PR005509 | 160-40 | 1.46E−07 | 1.89E+05 | 2.75E−02 | 0.9912 |
| | PR005512 | 160-40 | 5.33E−08 | 2.32E+05 | 1.24E−02 | 0.9939 |
| | PR005514 | 160-40 | 2.72E−07 | 2.24E+05 | 6.09E−02 | 0.9844 |
| | PR006475 | 160-40 | Not detectable | | | |
| | PR006480 | 240-60 | 1.04E−07 | 6.85E+04 | 7.13E−03 | 0.9857 |

### Example 5. Detection of the Binding of Anti-CD200R1 Antibodies to CHOK1/hD200R1a and CHOK1/cynoCD200R1a by FACS

This example is intended to investigate the in vitro binding activity of human anti-CD200R1 monoclonal antibodies to human/Cynomolgus macaques CD200R1a expressed on the surface of cells. The anti-CD200R1 antibodies were produced as described in Example 3.4 and the binding experiments at the cell level were performed using CHOK1/hCD200R1a and CHOK1-cynoCD200R1a.

Briefly, CHOK1/hCD200R1a cells and CHOK1-cynoCD200R1a cells were digested, and collected, and then were resuspended in PBS containing 2% BSA, respectively. The cell density was adjusted to 1×10⁶ cells/mL. The cells were seeded in a 96-well V-bottom plate (Corning, #3894) at 100 μL/well, followed by the addition of test antibodies and reference antibodies in a series diluted concentration, each at 100 μL/well. The cells were incubated away from light at 4° C. for 1 h. Thereafter, the cells in each well were rinsed twice with 100 μL of pre-cooled PBS containing 2% BSA, and centrifuged at 500 g at 4° C. for 5 min, and then the supernatant was discarded. 100 μL of fluorescent secondary antibody (Alexa Fluor 488-conjugated AffiniPure Goat Anti-Human IgG, Fcγ Fragment Specific, Jackson, #109-545-098, diluted in a 1:1000 ratio) was added in each well, and the plate was incubated away from light at 4° C. for 1 h. The cells in each well were rinsed twice with 100 μL of pre-cooled PBS containing 2% BSA, and centrifuged at 500 g at 4° C. for 5 min, and then the supernatant was discarded. Finally, the cells in each well were resuspended in 200 μL of pre-cooled PBS containing 2% BSA, and the fluorescence signal values were read using a BD CantoII flow cytometer.

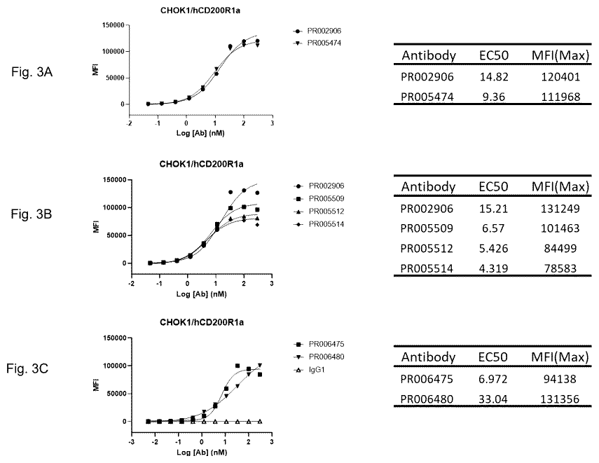

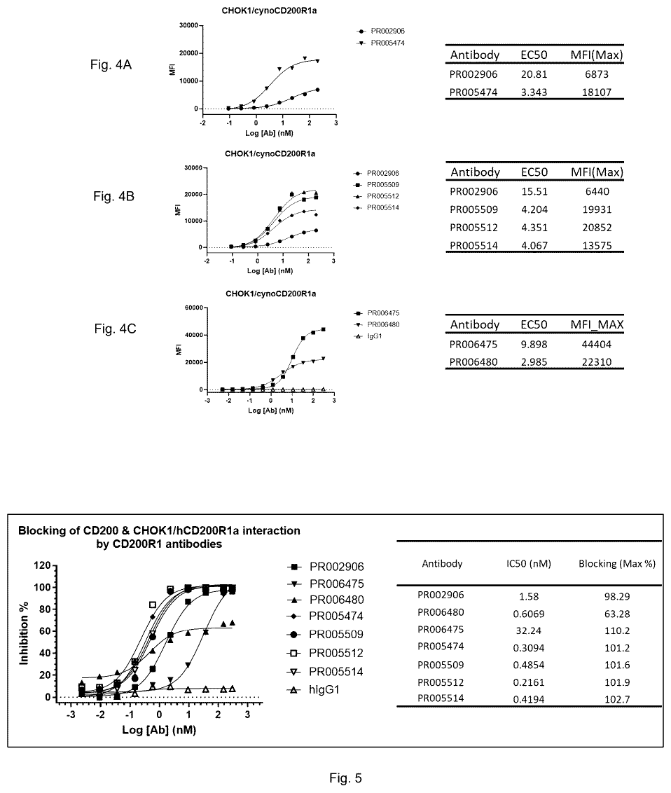

The results of the binding of the antibodies to CHOK1/hCD200R1a are shown in FIG. 3 (FIGS. 3A-3C). The anti-CD200R1 antibodies PR005474, PR005509, PR005512, PR005514 and PR006475 have stronger binding activity to human CD200R1a expressed on the surface of CHOK1 cell line in vitro, as compared to the reference antibody PR002906. The results of the binding of the antibodies to CHOK1/cynoCD200R1a are shown in FIG. 4 (FIGS. 4A-4C). The anti-CD200R1 antibodies PR005474, PR005509, PR005512, PR005514, PR006475 and PR006480 show stronger cross-binding activity to cynomolgus macaques CD200R1a expressed on the surface of CHOK1 cell line in vitro, as compared to the reference antibody PR002906.

### Example 6. Evaluation of the Blocking Effect of Anti-CD200R1 Antibodies on the Binding of Human CD200 Protein to CHOK1/hCD200R1a

To study the activity of human anti-CD200R1 antibodies in blocking the binding of human CD200 to its receptor CD200R1a in vitro, CHOK1/hCD200R1a was used to perform cell-level human CD200/human CD200R1a binding and blocking experiments.

In brief, CHOK1/hCD200R1a cells were digested and resuspended in 2% BSA in PBS. The cell density was adjusted to 1×10⁶ cells/mL, and the cells were inoculated on a 96-well V-bottom plate (Corning, Cat #: 3894) in 100 μL cells/well, followed by the addition of test antibodies and reference antibodies in a series diluted concentration, each at 100 μL/well, and hIgG1 was used as a control. The cells were placed at 4° C. and incubated for 1 hour in the dark. After that, centrifugation was performed at 4° C. for 5 minutes, the supernatant was discarded, and then 1 μg/mL biotin-labeled human CD200 protein (Acro Biosystems, B77-H82F5) was added at 50 μL/well, and incubation was performed at 4° C. in the dark for 30 minutes. 100 μL/well of pre-cooled PBS containing 2% BSA was added to rinse the cells twice, centrifugation was performed at 500 g, 4° C. for 5 minutes, and the supernatant was discarded. Fluorescent secondary antibody (PE Streptavidin, BD, Cat #: 554061, 1:200) was added at 100 μL/well, and incubation was performed at 4° C. for 30 minutes in the dark. The cells were washed twice with 200 μL/well of pre-cooled PBS, and were centrifuged at 500 g, 4° C. for 5 minutes, and the supernatant was discarded. Finally, 200 μL/well of pre-cooled PBS was used to resuspend the cells, and the fluorescence signal values were read using ACEA Novocyte 3000 flow cytometer. IC50 values were calculated.

The results are shown in FIG. 5. The antibodies of the invention, PR005474, PR005509, PR005512, PR005514 and PR006480, show superior blocking efficiency, as compared to PR006475 and the reference antibody PR002906.

### Example 7: Evaluation of the Inhibition of CD200-Mediated CD200R1a Signaling by Anti-CD200R1 Antibodies

The PathHunter® Jurkat CD200R1a signaling cell line expressing CD200R1a on the surface of the cell was purchased from Eurofins DiscoverX Products LLC (Cat No. 93-1136C19), and was used for the detection of the interaction between human CD200 and its receptor CD200R1. The binding of CD200 to CD200R1a expressed on the reporter cells (PathHunter® Jurkat CD200R1 signaling cells) leads to assembly of β-galactosidase and a subsequent luminescent readout. Blocking the CD200-CD200R1a interaction by anti-CD200R1 antibodies will decrease or abrogate the luminescent signal.

The cDNA encoding human CD200 (NP_005935.4, SEQ ID NO:185) was obtained by gene synthesis and was cloned into a lentiviral vector pGWLV11(AZENTA Life sciences). The lentiviral particles were generated and transfected CHOK1 cells to construct CHOK1/huCD200 cells that highly express human CD200 (AZENTA Life sciences).

Specifically, PathHunter® Jurkat CD200R1 signaling cells were collected and resuspended at 8×10⁵ cells/ml, then 25 μl of the cells were inoculated into VieePlate 96-well plate (PerkinElmer, Cat #6005181). CHOK1/hCD200 cells were digested and resuspended at 2×10⁶ cells/ml, and were inoculated into the plate at 25 μL cells/well. Anti-CD200R1 antibodies (the antibodies of the invention PR005474, PR005509, PR005512, PR005514, PR006475, and PR006480, and the reference antibody PR002903, as well as the control antibody IgG1) were serially diluted and were added into the plate at 100 μl/well respectively. Then, the plate was incubated at room temperature for 2 hours. After incubation, PATTHUNTER®Flash Detection Kit reagent was added and incubated for additional 30 min. Luminescence was measured on Envision plate reader (PerkinElmer, Model 2105).

The results are shown in FIG. 6 (FIG. 6A and FIG. 6B). The antibodies PR005474, PR005509, PR005512, PR005514 and PR006480 show superior blocking efficiency as compared to the reference anti-CD200 antibody PR002903.

### Example 8. Maturation of the Humanized Anti-CD200R1 Antibodies PR006480 and PR005514

### 8.1 Elimination of ADCC Effector Function by Mutation in the Fc Region

The nucleic acid sequence encoding the heavy chain variable region (VH) of the antibody was genetically synthesized and cloned into pTT5 mammalian cell expression vector comprising the nucleic acid sequence encoding the heavy chain constant domain of human IgG1 antibody, and L234A, L235A and G237A mutations (substitution of leucine with alanine at positions 234 and 235 and substitution of glycine with alanine at position 237 according to EU numbering) were introduced in the CH2 region of the heavy chain constant region of the IgG1 to eliminate the ADCC effector function. The plasmid encoding the heavy chain and the plasmid encoding the light chain of the antibody, respectively, were then transfected into CHOK1 or HEK 293 mammalian host cell lines by the method described in Example 3.4 to obtain the recombinant antibody protein.

The antibody sequences of PR006895 and PR006839 derived from PR006480 and PR005514 respectively are listed in Table 6.

TABLE 6
Antibodies obtained by Fc mutation of PR006480 and PR005514

| | | | | Light | Heavy | | | | | | | |
|---|---|---|---|---|---|---|---|---|---|---|---|---|
| | | | | chain | chain | Light | Light | Light | Heavy | Heavy | Heavy | |
| | Parental | Light | Heavy | variable | variable | chain | chain | chain | chain | chain | chain | Fc |
| Antibody | antibody | chain | chain | region | region | CDR1 | CDR2 | CDR3 | CDR1 | CDR2 | CDR3 | mutation |
| PR006839 | PR005514 | 158 | 147 | 124 | 111 | 62 | 76 | 92 | 11 | 26 | 46 | L234A, |
| | | | | | | | | | | | | L235A, |
| | | | | | | | | | | | | G237A |
| PR006895 | PR006480 | 162 | 148 | 128 | 114 | 65 | 79 | 96 | 11 | 29 | 49 | L234A, |
| | | | | | | | | | | | | L235A, |
| | | | | | | | | | | | | G237A |

### 8.2 Sequence Analysis and Sequence Optimization of Antibodies

In this example, amino acid mutations were introduced into the potential post-translational modification (PTM) sites in the sequences of the antibodies PR006895 and PR006839 to obtain the new antibody molecules (referred to as PTM variants). The amino acid sequences of the light and heavy chain variable domains, the light chain, the heavy chain (human IgG1) and the CDRs defined according to the Chothia scheme of the PTM variants in this example are listed in Table 7. All designed PTM variants were purified by the method described in Example 3.4 to obtain the purified recombinant antibodies, and further validated in subsequent functional experiments.

TABLE 7
Antibodies obtained after PTM mutations

| | | | | Light | Heavy | | | | | | |
|---|---|---|---|---|---|---|---|---|---|---|---|
| | | | | chain | chain | Light | Light | Light | Heavy | Heavy | Heavy |
| Initial | PTM | Light | Heavy | variable | variable | chain | chain | chain | chain | chain | chain |
| antibody | variant | chain | chain | region | region | CDR1 | CDR2 | CDR3 | CDR1 | CDR2 | CDR3 |
| PR006895 | PR007221 | 163 | 149 | 129 | 115 | 65 | 79 | 97 | 11 | 30 | 49 |
| | PR007223 | 164 | 150 | 130 | 116 | 65 | 79 | 98 | 11 | 31 | 50 |
| | PR007442 | 165 | 149 | 131 | 115 | 65 | 79 | 97 | 11 | 30 | 49 |
| | PR007444 | 166 | 150 | 132 | 116 | 65 | 79 | 98 | 11 | 31 | 50 |
| PR006839 | PR007630 | 167 | 151 | 133 | 117 | 66 | 80 | 92 | 11 | 32 | 46 |
| | PR007631 | 168 | 152 | 134 | 118 | 66 | 81 | 92 | 11 | 33 | 46 |
| | PR007972 | 169 | 153 | 135 | 119 | 67 | 76 | 99 | 14 | 32 | 46 |
| | PR007973 | 170 | 151 | 136 | 117 | 67 | 80 | 99 | 11 | 32 | 46 |
| | PR007974 | 171 | 154 | 137 | 120 | 66 | 80 | 100 | 14 | 33 | 46 |

### Example 9. Measurements of the Binding Affinity of the PTM Variants Derived from PR006480 and PR005514 to CD200R1a

The methods for determining binding affinities of the PTM variants derived from PR006480 and PR005514 to human CD200R1a and to cyno CD200R1a recombinant protein are similar to Example 4. The data of binding affinity of the anti-CD200R1 antibodies to human CD200R1a and cyno CD200R1a are summarized in Table 8. The results indicate that PR007223, PR007973 and PR007221 have high binding affinities to human CD200R1a and cyno CD200R1a, with no binding to mouse CD200R1.

TABLE 8
Binding Affinity of Variants derived from PR006480 and PR005514

| Antibodies | Analyte | KD (M) | kon(1/Ms) | kdis(1/s) | Full R\hat{ }2 |
|---|---|---|---|---|---|
| PR007223 | Human CD200R1a | 2.07E−09 | 6.42E+04 | 1.33E−04 | 0.9985 |
| PR007973 | Human CD200R1a | 1.06E−09 | 5.62E+04 | 5.97E−05 | 0.9989 |
| PR007221 | Human CD200R1a | 4.34E−09 | 4.86E+04 | 2.11E−04 | 0.9989 |
| PR007223 | Cyno CD200R1a- | 1.63E−08 | 8.60E+04 | 1.40E−03 | 0.9877 |
| | His | | | | |
| PR007973 | Cyno CD200R1a- | 1.02E−08 | 1.49E+04 | 1.51E−04 | 0.9954 |
| | His | | | | |
| PR007221 | Cyno CD200R1a- | 1.25E−08 | 6.21E+04 | 7.76E−04 | 0.9946 |
| | His | | | | |
| PR007223 | Mouse CD200R1- | No Binding | | | |
| | His | | | | |
| PR007973 | Mouse CD200R1- | No Binding | | | |
| | His | | | | |
| PR007221 | Mouse CD200R1 | No Binding | | | |
| | His | | | | |

### Example 10. Evaluation of Binding Epitope Competition of the PTM Variants Derived from PR006480 and PR005514

To determine if the anti-CD200R1 antibodies bind to human CD200R1a on different or approximate binding epitopes, epitope competition experiments for the anti-CD200R1 antibodies were performed by the ForteBlio Octet® RED96e platform. Human CD200R1a protein was diluted to 3 μg/ml with the kinetic buffer (10× kinetic buffer (Catalog #18-1105, ForteBlio)), and then loaded onto anti-Penta IS biosensors (Catalog #18-5120, ForteBlio) to reach capture levels to 0.3 nm. The in-tandem competition assay format was applied, and it contained two association steps. Firstly, the antigen-loaded biosensors bound to each antibody (i.e., First antibody, 1^{st} Ab) at a saturating concentration of 400 nM for 300 seconds to reach equilibrium and then secondly bound to the competing antibodies (i.e., Second antibody, 2^{nd} Ab) of 400 nM for 300 seconds. The second binding signals are recorded as the 100% signal of each antibody when the first antibodies are replaced by kinetic buffer. All the binding data were analyzed using ForteBlio Data Analysis 11.0 software. The inhibition rate was calculated by the following formula:

$$Inhibition rate ( \% ) = ( A - B ) / A * 100 ,$$

the “A” represents 100% signal of each antibody; and
the “B” represents the signals of second antibody binding steps.

If the obtained inhibition rate is greater than 80(%), it indicates that the epitopes of the two antibodies completely overlap; if the inhibition rate is less than 40(%), it indicates that the epitopes of the two antibodies are different or far away from each other.

The results in Table 9 show that the antibodies PR007223, PR007973 and PR007221 significantly compete with each other, indicating that they may share the same binding epitope of human CD200R1a. In addition, the reference antibody PR006207 did not block the antigen-binding of other antibodies no matter it served as 1^{st} Ab or 2^{nd} Ab, which suggests that PR006207 binds to the epitope of the human CD200R1a which is different from that bound by PR007223, PR007973 and PR007221.

TABLE 9
The inhibition rates of antibodies in the epitope competition assay

| | | 2^{nd} Ab | | | |
|---|---|---|---|---|---|
| Inhibition ratio (%) | | PR007223 | PR007973 | PR007221 | PR006207 |
| 1^{st} Ab | PR007223 | 91.80% | 99.63% | 101.32% | −50.24% |
| | PR007973 | 91.70% | 102.67% | 100.27% | −44.66% |
| | PR007221 | 88.95% | 95.58% | 100.41% | −40.69% |
| | PR006207 | −38.64% | −39.93% | −36.16% | 97.56% |

### Example 11. Detection of the Binding of the Anti-CD200R1 Antibody Variants to CHOK1/hCD200R1a and CHOK1-cynoCD200R1a by FACS

This example is intended to investigate the in vitro binding activity of the human anti-CD200R1 monoclonal antibodies, which have been engineered to eliminate ADCC effector function and remove PTMs, to human/Cynomolgus macaques CD200R1a. Antibody binding experiments at the cell level were described as the Example 5.

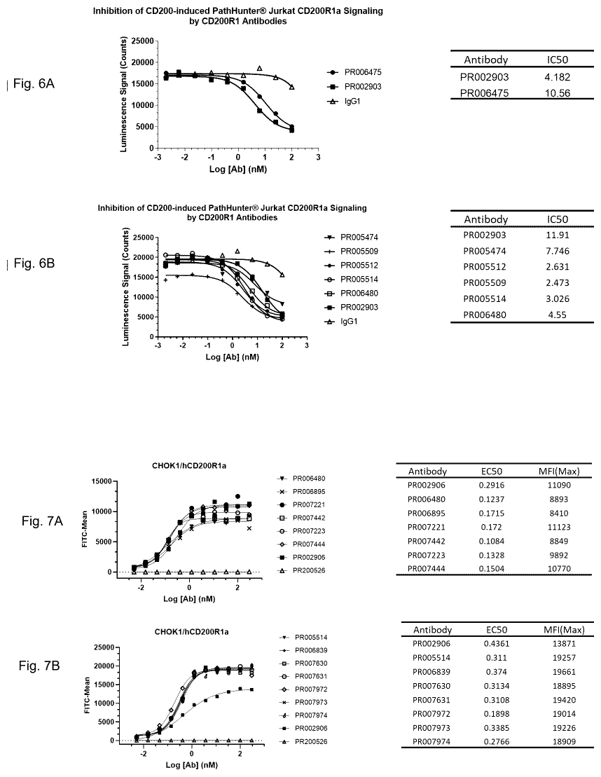

The results of the binding of the anti-CD200R1 antibodies to CHOK1/hCD200R1a cells are shown in FIG. 7 (FIG. 7A and FIG. 7B). The variant antibodies PR007221, PR007223, PR007442, and PR007444 have stronger binding activities as compared to the reference antibody PR002906, and have comparable or stronger binding activities as compared to their parental antibodies i.e., PR006480 and PR006895. The variant antibodies PR007630, PR007631, PR007972, PR007973 and PR007974 have stronger binding activities as compared to the reference antibody PR002906, and have comparable or stronger binding activities as compared to their parental antibodies, i.e., PR005514 and PR006839.

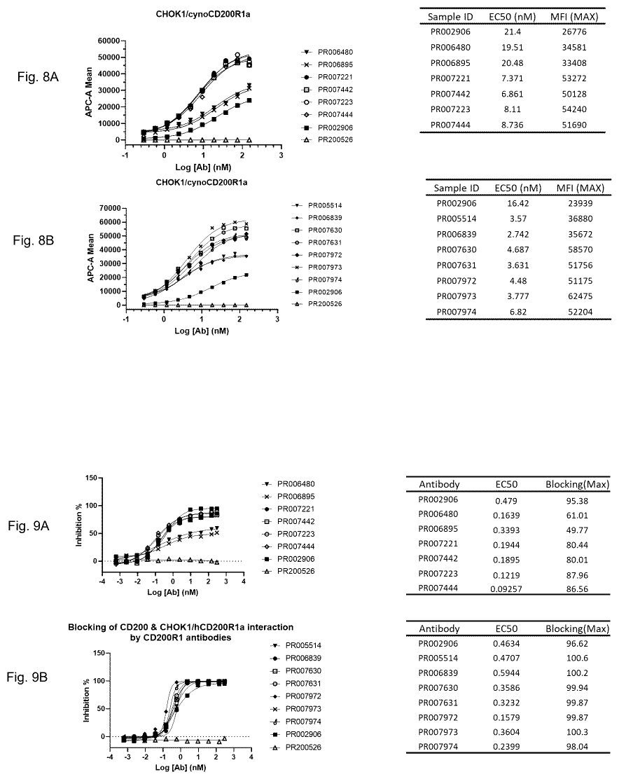

The results of the binding of these antibodies to CHOK1/cynoCD200R1a are shown in FIG. 8 (FIG. 8A and FIG. 8B). The variant antibodies PR007221, PR007223, PR007442, PR007444, PR007630, PR007631, PR007972, PR007973 and PR007974 show better cross-binding activities to Cynomolgus macaques CD200R1a than the reference antibody PR002906. The variant antibodies PR007221, PR007223, PR007442, and PR007444 have stronger binding activities as compared to their parental antibodies i.e., PR006480 and PR006895. The variant antibodies PR007630, PR007631, PR007972, PR007973 and PR007974 have comparable binding activity as compared to their parental antibodies i.e., PR005514 and PR006839.

### Example 12. Evaluation of the Blocking Effect of the Variants of CD200R1 Antibodies on the Binding of Human CD200 Protein to CHOK1/hCD200R1a

To study the activity of human anti-CD200R1 antibodies, which have been engineered to eliminate ADCC effector function and remove PTMs, in blocking the binding of human CD200 to its receptor CD200R1a in vitro, antibody blocking experiments at the cell level as described in Example 6 were performed with the antibody variants.

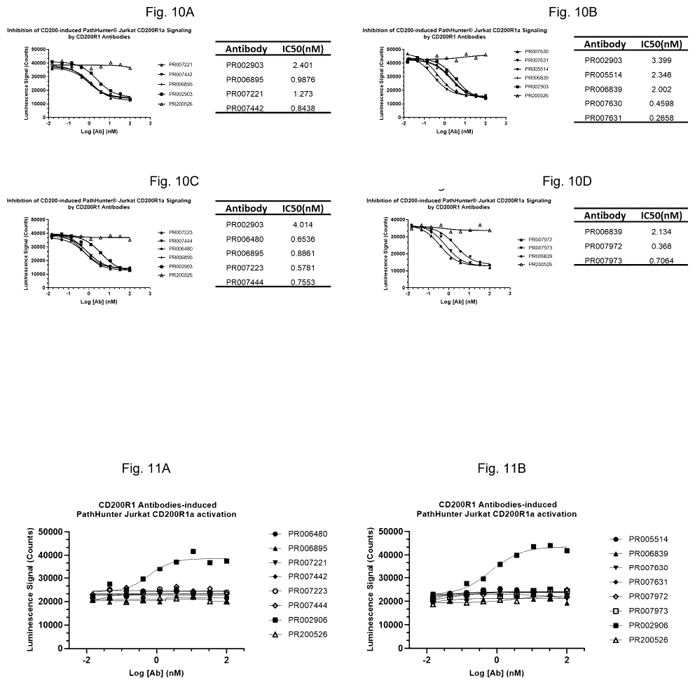

The results in FIG. 9 (FIG. 9A and FIG. 9B) show that the variant antibodies PR007221, PR007223, PR007442, PR007444, PR007630, PR007631, PR007972, PR007973 and PR007974 show better blocking activities than the reference antibody PR002906.

### Example 13: Evaluation of the Inhibition of CD200-Mediated CD200R1a Signaling by the Variant Anti-CD200R1 Antibodies

The PathHunter® Jurkat CD200R1a reporter cells were used to explore the antagonistic activity of anti-CD200R1 antibodies as described in Example 7. The results are shown in FIG. 10 (FIGS. 10A-10D).

As shown in the results, the variant antibodies PR007221, PR007223, PR007442, PR007444, PR007630, PR007631, PR007972, PR007973 and PR007974 show superior blocking activities than the reference antibody PR002903, as well as their parental antibodies, PR005514 and PR006839, PR006480 and PR006895 respectively.

### Example 14: Evaluation of the Activation of CD200R1a Signaling by Variants of Anti-CD200R1 Antibodies

This example is intended to investigate whether the variant anti-CD200R1 antibodies of the invention have any agonistic activities by using the PathHunter® Jurkat CD200R1 reporter cells.

Briefly, PathHunter® Jurkat CD200R1 cells were collected and resuspended at 8×10⁵ cells/ml, then were inoculated at 25 μL cells/well into VieePlate 96-well plate (PerkinElmer, Cat #6005181). CHOK1-hCD32b (BPS Biosciences, Cat #79511, FcGR2B-CHO K1) cells were digested and resuspended at 2×10⁶ cells/ml and were inoculated at 25 μL cells/well. The anti-CD200R1 antibodies were serially diluted and were added into the plate at 100 μl/well. PR200526 was used as a negative control. Then, the plate was incubated at room temperature for 2 hours. After incubation, PATHHUNTER®Flash Detection Kit reagent was added and incubated for additional 30 min. Luminescence was measured on Envision plate reader (PerkinElmer, Model 2105).

The results in FIG. 11 (FIG. 11A and FIG. 11B) show that the parental antibodies PR6480, PR005514 and the variant antibodies PR006895, PR007221, PR007442, PR007223, PR007444, PR006839, PR007630, PR007631, PR007972, and PR007973 didn't show any agonistic activity comparing to the reference antibody PR002906.

### Example 15. Evaluation of the Cross-Reactivity of Anti-CD200R1 Antibodies with CD200R1L

To test whether the antibodies have cross-reactivity with CD200R1L, binding of these antibodies to CD200R1L was tested.

In specific, the cell surface glycoprotein CD200 receptor 2 isoform 1 precursor CD200R1L recombinant protein (Sino Biologicals, 11620-H08H, SEQ ID NO:174) were diluted with PBS to 1 μg/mL, and added to a 96-well plate (Corning, #9018) at 100 μl per well, and incubated overnight at 4° C. After the liquid was discarded, the plate was washed 3 times with PBST buffer (pH 7.4, containing 0.05% tween-20), 250 μl 2% BSA blocking solution was added into the plate and incubated at 37° C. for 1 hour. The blocking solution was discarded and the plate was washed 3 times with PBST buffer (pH 7.4, containing 0.05% Tween-20). The anti-CD200R1 antibodies were diluted and added at 100 l/well, incubated at 37° C. for 1 hour, with isotype antibody as a control. After the plate was washed 3 times with PBST buffer (pH 7.4, containing 0.05% Tween-20), goat anti-human HRP secondary antibody (Invitrogen, #A18805) was diluted 5000 times, added into the plate and incubated for 1 hour at 37° C. in the dark. After the plate was washed with PBST buffer (pH 7.4, containing 0.05% tween-20) for 3 times, 100p/well of TMB (Biopanda, #TMB-S-003) was add and the plate was placed in the dark at room temperature for about 30 minutes; stop solution (BBI life sciences, #E661006-0200) was added at 50p/well to each well to terminate the reaction, and the absorbance (OD450) at 450 nm was measured in a microplate reader (PerkinElemer, #Enspire).

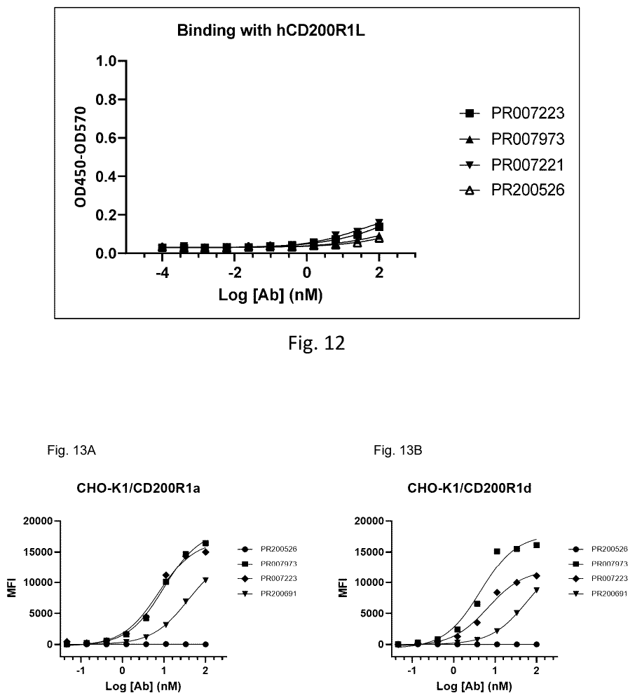

The results in FIG. 12 illustrate that the anti-CD200R antibodies PR007223, PR007973 and PR007221 does not cross-react with CD200R1L.

### Example 16. Detection of the Binding of Anti-CD200R1 Antibodies to CHOK1/hCD200R1a and CHOK1/hCD200R1d by FACS

This example is intended to investigate the in vitro binding activity of the variant anti-CD200R1 monoclonal antibodies to CHOK1/hCD200R1a and CHOK1/hCD200R1d. Briefly, the CD200R1 antibodies (PR007973 and PR007223) and the reference antibodies (PR200526 and PR200691) were labelled with Alexa Fluor 647 by using the protein labeling kit (Invitrogen, A20173). CHOK1/hCD200R1a cells and CHOK1/hCD200R1d cells were digested, and collected, and then these cells were resuspended in PBS containing 2% BSA, respectively. The cell density was adjusted to 1×10⁶ cells/mL. The binding of anti-CD200R1 antibodies were analyzed as described in Example 5.

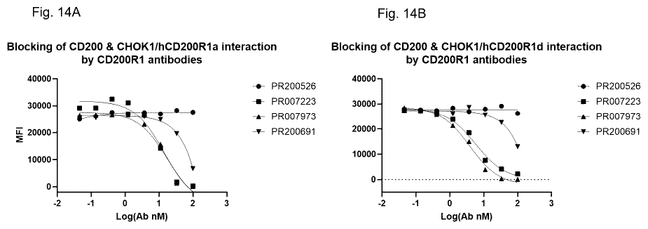

The results are shown in FIG. 13 (FIG. 13A and FIG. 13B). Comparing to PR200691 reference antibody, PR007973 shows high binding activity to both CD200R1 isoform a and isoform d. PR007223 shows high binding activity to CD200R1 isoform a while weaker binding to CD200R1 isoform d.

### Example 17. Evaluation of the Blocking Effect of the Anti-CD200R1 Antibodies on the Binding of Human CD200 Protein to CHOK1/hCD200R1a and CHOK1/hCD200R1d

To study the activity of human anti-CD200R1 antibodies in blocking the binding of human CD200 to its receptor CD200R1a and CD200R1d in vitro, CHOK1/CD200R1a and CHOK1/CD200R1d were used to perform cell-level human CD200/human CD200R1 binding and blocking experiments as described in Example 6.

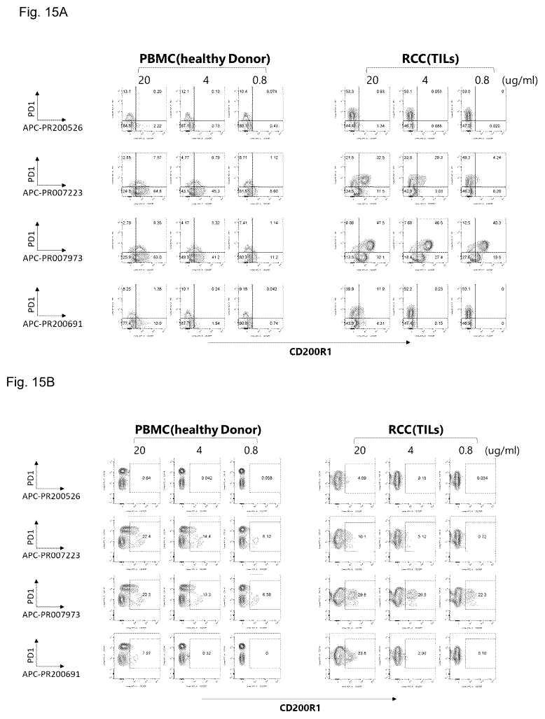

The results are shown in FIGS. 14A and 14B. Comparing to PR200691 reference antibody, both PR007223 and PR007973 show superior blocking activity.

### Example 18: Evaluation of the Binding of Anti-CD200R1 Antibodies to Immune Cells in Tumor Microenvironment

To explore the binding of anti-CD200R1 antibodies to tumor infiltrating lymphocytes (TILs), human CD200R1 antibodies were labelled with Alexa Fluor 647 by using the protein labeling kit (Invitrogen, A20173). The binding of CD200R1 antibodies to PBMCs and dissociated renal cell carcinoma single cell suspension was analyzed as Example 1.

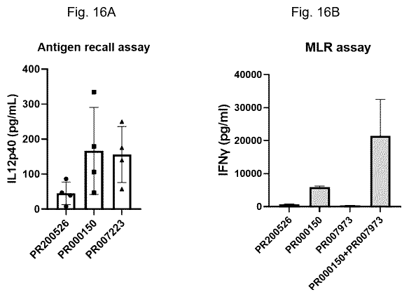

The results are shown in FIG. 15. Comparing to PR200691 reference antibody, PR007973 shows high binding affinity to PD1-positive T cells (FIG. 15A) and superior binding to CD11b-positive myeloid cells from both PBMCs and renal cell carcinoma patient (FIG. 15B). PR007223 shows high binding affinity to PBMCs from health donor, and binds to PD1-positive T cells and CD11b-positive myeloid cells from both PBMCs and renal cell at high concentration (20 g/ml).

### Example 19: Functional Activities of Anti-CD200R1 Antibodies on Human Primary Cells

Functional activity of anti-CD200R1 antibodies to inhibit the CD200-CD200R1 signaling was tested by using antigen recall assay and mixed lymphocyte reaction assay (MLR). In the antigen recall assay, PBMCs from four different healthy HLA-A2+ donors were isolated and incubated with 100 ng/ml IL4, 100 ng/ml IL10 and 100 ng/ml M-CSF for 72 h. The cells are washed and resuspended in culture medium. Anti-CD200R1 antibodies PR007223, PR000150 (anti-PD1 antibody, Pembrolizumab analog, HC/LCSEQ ID NO 183 and 184, produced as described in Example 4) and isotype control PR200526 were added at final concentration 50 nM. CEF peptide (JPT, PM-CEF-E) was added at a final concentration of 1 g/ml. Cells were cultured for another 7 days. Supernatants were collected and IL12/IL23p40 was detected by MSD (Meso Scale Discovery, MSD QuickPlex SQ120).

In the MLR assay, monocytes were isolated from human PBMCs by using monocyte isolation kit (STEMCELL, Cat No. 19359) were cultured with 50 ng/ml IL4 and 50 ng/ml M-CSF for 6 days. Cells were collected as mono-DCs. T cells were isolated from PBMCs from a different healthy donor by using human T cell isolation kit (STEMCELL, Cat No. 17951). Mono-DCs and T cells were mixed together, anti-CD200R1 antibody PR007973 and PR000150 were added at final concentration 50 nM. Cells were cultured for another 4 days. Supernatants were collected and IFNg was detected by MSD (Meso Scale Discovery, MSD QuickPlex SQ120).

The results are shown in FIG. 16 (FIG. 16A and FIG. 16B). Anti-CD200R1 antibody PR007223 greatly enhanced IL12p40 production as compared to isotype control in the antigen recall assay. PR007973 can greatly increase the IFNg production in the presence of anti-PD1 antibody PR000150.

### Example 20. Evaluation of the Anti-Tumor Efficacy of Anti-CD200R1 Antibody in CD200 Positive Tumors

To explore the anti-tumor activity of anti-CD200R1 antibodies, CD200 positive tumor cell lines, the human lung squamous carcinoma cell line NCI-H226 (ATCC, CRL-5826) and the human ovarian cell line OVCAR3 (ATCC, HTB-161) CDX models in humanized NCG mice (Gempharmatech Co) were used for the in vivo studies.

On the day of tumor cell inoculation, NCI-H226 tumor cells were resuspended in the mixed solution of PBS and Matrigel (1:1). Each NCG mouse was subcutaneously inoculated with 5×10⁶ NCI-H226 cells. When the average tumor volume of mice reached about 90 mm³, mice were randomized into 6 mice/group. Then, each mouse was intravenously inoculated with 5×10⁶ human PBMC. Next day, the anti-CD200R1 antibodies (PR005514 and PR006480), the negative control antibody IgG1, and the positive anti-CD200 antibody PR002903 were administered at 20 mg/kg via the tail vein twice a week for a total of 4 weeks respectively. The tumor volume and body weight were measured twice a week. The tumor volume was calculated according to the following formula:

$$tumor volume ( {mm}^{3} ) = 0.5 \times tumor long diameter \times tumor short {diameter}^{2} .$$

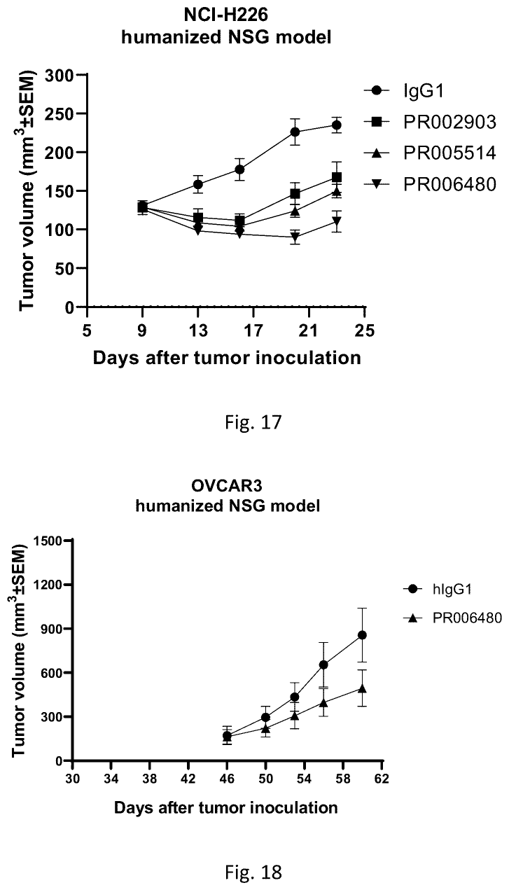

The anti-tumor effect of anti-CD200R1 antibodies in the NCI-H226 CDX model in humanized NCG mice is shown in FIG. 17. Comparing to the positive control anti-CD200 antibody PR002903 that shows 35.3% tumor growth inhibition (TGI) rate at day 20 after treatment, the anti-CD200R antibody PR005514 shows 45.2% TGI and PR006480 showed 60.2% TGI.

In the second experiment, OVCAR3 cells were resuspended in the mixed solution of PBS and Matrigel (1:1). Each NCG mouse was subcutaneously inoculated with 5×10⁶ OVCAR3 cells. When the average tumor volume of mice reached about 90 mm³, mice were randomized into 6 mice/group. Then, each mouse was intravenously inoculated with 5×10⁶ human PBMC. Next day, the anti-CD200R1 antibody (PR006480) and the negative control hIgG1 were administered at 20 mg/kg via the tail vein twice a week for a total of 3 weeks respectively. The tumor volume and body weight were measured twice a week. The tumor volume was calculated according to the following formula:

$$tumor volume ( {mm}^{3} ) = 0.5 \times tumor long diameter \times tumor short {diameter}^{2} .$$

The anti-tumor effect of anti-CD200R1 antibody in the OVCAR3 CDX model in humanized NCG mice is shown in FIG. 18. As shown in the results, PR006480 shows 42.4% tumor inhibition rate TGI at day 13 after treatment.

### Example 21. Evaluation of the Anti-Tumor Efficacy of Anti-CD200R1 Antibody in CD200 Negative Tumors

To explore the anti-tumor activity of anti-CD200R1 antibodies in CD200 negative tumor cell lines, the human lung cancer cell line NCI-H292 (ATCC, CRL-1848) and the human breast cancer cell MDA-MB-231 (ATCC, HTB-26) CDX models in humanized NCG mice (Gempharmatech Co) were used for the in vivo studies.

On the day of tumor cell inoculation, NCI-H292 tumor cells were resuspended in the mixed solution of PBS and Matrigel (1:1). Each NCG mouse was subcutaneously inoculated with 5×10⁶ NCI-H292 cells. When the average tumor volume of mice was about 90 mm³, mice were randomized into 6 mice/group. Then, each mouse was intravenously inoculated with 5×10⁶ human PBMC. Next day, anti-CD200R1 antibodies (PR005514, PR005512, PR005509, with PBS as a negative control) were administered at 20 mg/kg via the tail vein twice a week for a total of 4 weeks respectively. The tumor volume and body weight were measured twice a week. The tumor volume was calculated according to the following formula:

$$tumor volume ( {mm}^{3} ) = 0.5 \times tumor long diameter \times tumor short {diameter}^{2} .$$

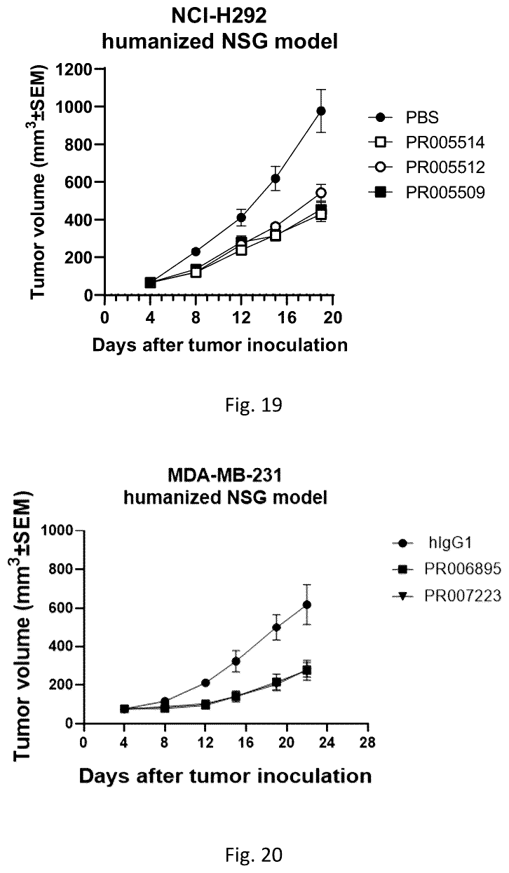

The anti-tumor effect of anti-CD200R1 antibodies in the NCI-H292 CDX model in humanized NCG mice is shown in FIG. 19. It is indicated that 14 days after anti-CD200R antibodies treatment, PR005514 shows 56.2% TGI value; PR005512 shows 44.5% TGI; and PR005509 shows 53.6% TGI.

In the MDA-MB-231 CDX model, MDA-MB-231 tumor cells were resuspended in the mixed solution of PBS and Matrigel (1:1). Each NCG mouse was subcutaneously inoculated with 5×10⁶ MDA-MB-231 cells. When the average tumor volume of mice was about 90 mm³, mice were randomized into 6 mice/group. Then, each mouse was intravenously inoculated with 5×10⁶ human PBMC. Next day, anti-CD200R1 antibodies (i.e., the antibodies of the invention PR006895, PR007223 and the negative control antibody hIgG1) were administered at 20 mg/kg via the tail vein twice a week for a total of 4 weeks respectively. The tumor volume and body weight were measured twice a week. The tumor volume was calculated according to the following formula:

$$tumor volume ( {mm}^{3} ) = 0.5 \times tumor long diameter \times tumor short {diameter}^{2} .$$

The anti-tumor effect of anti-CD200R1 antibodies in the MDA-MB-231 CDX model in humanized NCG mice is shown in FIG. 20. It is indicated that 17 days after anti-CD200 antibodies treatment, PR006895 shows 55.5% tumor inhibition rate TGI and PR007223 shows 55.0% TGI.

1. An antibody that specifically binds to CD200R1, or an antigen binding fragment thereof, wherein the antibody comprises a light chain variable region (VL) and a heavy chain variable region (VH), and wherein
(1) the VH comprises the HCDRs 1-3 of a VH having the amino acid sequence set forth in SEQ ID NO: 117, and the VL comprises the LCDRs 1-3 of a VL having the amino acid sequence set forth in SEQ ID NO: 136; or
(2) the VH comprises the HCDRs 1-3 of a VH having the amino acid sequence set forth in SEQ ID NO: 111, and the VL comprises the LCDRs 1-3 of a VL having the amino acid sequence set forth in SEQ ID NO: 124; or
(3) the VH comprises the HCDRs 1-3 of a VH having the amino acid sequence set forth in SEQ ID NO: 117, and the VL comprises the LCDRs 1-3 of a VL having the amino acid sequence set forth in SEQ ID NO: 133; or
(4) the VH comprises the HCDRs 1-3 of a VH having the amino acid sequence set forth in SEQ ID NO: 118, and the VL comprises the LCDRs 1-3 of a VL having the amino acid sequence set forth in SEQ ID NO: 134; or
(5) the VH comprises the HCDRs 1-3 of a VH having the amino acid sequence set forth in SEQ ID NO: 119, and the VL comprises the LCDRs 1-3 of a VL having the amino acid sequence set forth in SEQ ID NO: 135; or
(6) the VH comprises the HCDRs 1-3 of a VH having the amino acid sequence set forth in SEQ ID NO: 120, and the VL comprises the LCDRs 1-3 of a VL having the amino acid sequence set forth in SEQ ID NO: 137; or
(7) the VH comprises the HCDRs 1-3 of a VH having the amino acid sequence set forth in SEQ ID NO: 108, and the VL comprises the LCDRs 1-3 of a VL having the amino acid sequence set forth in SEQ ID NO: 123; or
(8) the VH comprises the HCDRs 1-3 of a VH having the amino acid sequence set forth in SEQ ID NO: 109, and the VL comprises the LCDRs 1-3 of a VL having the amino acid sequence set forth in SEQ ID NO: 124; or
(9) the VH comprises the HCDRs 1-3 of a VH having the amino acid sequence set forth in SEQ ID NO: 110, and the VL comprises the LCDRs 1-3 of a VL having the amino acid sequence set forth in SEQ ID NO: 125; or
(10) the VH comprises the HCDRs 1-3 of a VH having the amino acid sequence set forth in SEQ ID NO: 114, and the VL comprises the LCDRs 1-3 of a VL having the amino acid sequence set forth in SEQ ID NO: 128; or
(11) the VH comprises the HCDRs 1-3 of a VH having the amino acid sequence set forth in SEQ ID NO: 115, and the VL comprises the LCDRs 1-3 of a VL having the amino acid sequence set forth in SEQ ID NO: 129; or
(12) the VH comprises the HCDRs 1-3 of a VH having the amino acid sequence set forth in SEQ ID NO: 116, and the VL comprises the LCDRs 1-3 of a VL having the amino acid sequence set forth in SEQ ID NO: 130; or
(13) the VH comprises the HCDRs 1-3 of a VH having the amino acid sequence set forth in SEQ ID NO: 115, and the VL comprises the LCDRs 1-3 of a VL having the amino acid sequence set forth in SEQ ID NO: 131; or
(14) the VH comprises the HCDRs 1-3 of a VH having the amino acid sequence set forth in SEQ ID NO: 116, and the VL comprises the LCDRs 1-3 of a VL having the amino acid sequence set forth in SEQ ID NO: 132.

2. The antibody that specifically binds to CD200R1, or the antigen binding fragment thereof according to claim 1, wherein
(1) the VL comprises LCDR1-3 having the amino acid sequences set forth in SEQ ID NOs: 67, 80, 99 respectively or LCDRs 1-3 having amino acid sequences each of which differ by one or two amino acid residues as the sequences set forth in SEQ ID NOs: 67, 80, 99 respectively, and the VH comprises HCDRs 1-3 having the amino acid sequences set forth in SEQ ID NOs: 11, 32, 46 respectively or HCDRs 1-3 having amino acid sequences each of which differ by one or two amino acid residues as the sequences set forth in SEQ ID NOs: 11, 32, 46 respectively; or
(2) the VL comprises LCDR1-3 having the amino acid sequences set forth in SEQ ID NOs: 62, 76, 92 respectively or LCDRs 1-3 having amino acid sequences each of which differ by one or two amino acid residues as the sequences set forth in SEQ ID NOs: 62, 76, 92 respectively, and the VH comprises HCDRs 1-3 having the amino acid sequences set forth in SEQ ID NOs: 11, 26, 46 respectively or HCDRs 1-3 having amino acid sequences each of which differ by one or two amino acid residues as the sequences set forth in SEQ ID NOs: 11, 26, 46 respectively; or
(3) the VL comprises LCDR1-3 having the amino acid sequences set forth in SEQ ID NOs: 66, 80, 92 respectively or LCDRs 1-3 having amino acid sequences each of which differ by one or two amino acid residues as the sequences set forth in SEQ ID NOs: 66, 80, 92 respectively, and the VH comprises HCDRs 1-3 having the amino acid sequences set forth in SEQ ID NOs: 11, 32, 46 respectively or HCDRs 1-3 having amino acid sequences each of which differ by one or two amino acid residues as the sequences set forth in SEQ ID NOs: 11, 32, 46 respectively; or
(4) the VL comprises LCDR1-3 having the amino acid sequences set forth in SEQ ID NOs: 66, 81, 92 respectively or LCDRs 1-3 having amino acid sequences each of which differ by one or two amino acid residues as the sequences set forth in SEQ ID NOs: 66, 81, 92 respectively, and the VH comprises HCDRs 1-3 having the amino acid sequences set forth in SEQ ID NOs: 11, 33, 46 respectively or HCDRs 1-3 having amino acid sequences each of which differ by one or two amino acid residues as the sequences set forth in SEQ ID NOs: 11, 33, 46 respectively; or
(5) the VL comprises LCDR1-3 having the amino acid sequences set forth in SEQ ID NOs: 67, 76, 99 respectively or LCDRs 1-3 having amino acid sequences each of which differ by one or two amino acid residues as the sequences set forth in SEQ ID NOs: 67, 76, 99 respectively, and the VH comprises HCDRs 1-3 having the amino acid sequences set forth in SEQ ID NOs: 14, 32, 46 respectively or HCDRs 1-3 having amino acid sequences each of which differ by one or two amino acid residues as the sequences set forth in SEQ ID NOs: 14, 32, 46 respectively; or
(6) the VL comprises LCDR1-3 having the amino acid sequences set forth in SEQ ID NOs: 66, 80, 100 respectively or LCDRs 1-3 having amino acid sequences each of which differ by one or two amino acid residues as the sequences set forth in SEQ ID NOs: 66, 80, 100 respectively, and the VH comprises HCDRs 1-3 having the amino acid sequences set forth in SEQ ID NOs: 14, 33, 46 respectively or HCDRs 1-3 having amino acid sequences each of which differ by one or two amino acid residues as the sequences set forth in SEQ ID NOs: 14, 33, 46 respectively; or
(7) the VL comprises LCDRs 1-3 having the amino acid sequences set forth in SEQ ID NOs: 62, 76, 91 respectively or LCDRs 1-3 having amino acid sequences each of which differ by one or two amino acid residues as the sequences set forth in SEQ ID NOs: 62, 76, 91 respectively, and the VH comprises HCDRs 1-3 having the amino acid sequences set forth in SEQ ID NOs: 10, 25, 44 respectively or HCDRs 1-3 having amino acid sequences each of which differ by one or two amino acid residues as the sequences set forth in SEQ ID NOs: 10, 25, 44 respectively; or
(8) the VL comprises LCDR1-3 having the amino acid sequences set forth in SEQ ID NOs: 62, 76, 92 respectively or LCDRs 1-3 having amino acid sequences each of which differ by one or two amino acid residues as the sequences set forth in SEQ ID NOs: 62, 76, 92 respectively, and the VH comprises HCDRs 1-3 having the amino acid sequences set forth in SEQ ID NOs: 11, 26, 45 respectively or HCDRs 1-3 having amino acid sequences each of which differ by one or two amino acid residues as the sequences set forth in SEQ ID NOs: 11, 26, 45 respectively; or
(9) the VL comprises LCDR1-3 having the amino acid sequences set forth in SEQ ID NOs: 62, 76, 93 respectively or LCDRs 1-3 having amino acid sequences each of which differ by one or two amino acid residues as the sequences set forth in SEQ ID NOs: 62, 76, 93 respectively, and the VH comprises HCDRs 1-3 having the amino acid sequences set forth in SEQ ID NOs: 11, 25, 45 respectively or HCDRs 1-3 having amino acid sequences each of which differ by one or two amino acid residues as the sequences set forth in SEQ ID NOs: 11, 25, 45 respectively; or
(10) the VL comprises LCDR1-3 having the amino acid sequences set forth in SEQ ID NOs: 65, 79, 96 respectively or LCDRs 1-3 having amino acid sequences each of which differ by one or two amino acid residues as the sequences set forth in SEQ ID NOs: 65, 79, 96 respectively, and the VH comprises HCDRs 1-3 having the amino acid sequences set forth in SEQ ID NOs: 11, 29, 49 respectively or HCDRs 1-3 having amino acid sequences each of which differ by one or two amino acid residues as the sequences set forth in SEQ ID NOs: 11, 29, 49 respectively; or
(11) the VL comprises LCDR1-3 having the amino acid sequences set forth in SEQ ID NOs: 65, 79, 97 respectively or LCDRs 1-3 having amino acid sequences each of which differ by one or two amino acid residues as the sequences set forth in SEQ ID NOs: 65, 79, 97 respectively, and the VH comprises HCDRs 1-3 having the amino acid sequences set forth in SEQ ID NOs: 11, 30, 49 respectively or HCDRs 1-3 having amino acid sequences each of which differ by one or two amino acid residues as the sequences set forth in SEQ ID NOs: 11, 30, 49 respectively; or
(12) the VL comprises LCDR1-3 having the amino acid sequences set forth in SEQ ID NOs: 65, 79, 98 respectively or LCDRs 1-3 having amino acid sequences each of which differ by one or two amino acid residues as the sequences set forth in SEQ ID NOs: 65, 79, 98 respectively, and the VH comprises HCDRs 1-3 having the amino acid sequences set forth in SEQ ID NOs: 11, 31, 50 respectively or HCDRs 1-3 having amino acid sequences each of which differ by one or two amino acid residues as the sequences set forth in SEQ ID NOs: 11, 31, 50 respectively; or
(13) the VL comprises LCDR1-3 having the amino acid sequences set forth in SEQ ID NOs: 65, 79, 97 respectively or LCDRs 1-3 having amino acid sequences each of which differ by one or two amino acid residues as the sequences set forth in SEQ ID NOs: 65, 79, 97 respectively, and the VH comprises HCDRs 1-3 having the amino acid sequences set forth in SEQ ID NOs: 11, 30, 49 respectively or HCDRs 1-3 having amino acid sequences each of which differ by one or two amino acid residues as the sequences set forth in SEQ ID NOs: 11, 30, 49 respectively; or
(14) the VL comprises LCDR1-3 having the amino acid sequences set forth in SEQ ID NOs: 65, 79, 98 respectively or LCDRs 1-3 having amino acid sequences each of which differ by one or two amino acid residues as the sequences set forth in SEQ ID NOs: 65, 79, 98 respectively, and the VH comprises HCDRs 1-3 having the amino acid sequences set forth in SEQ ID NOs: 11, 31, 50 respectively or HCDRs 1-3 having amino acid sequences each of which differ by one or two amino acid residues as the sequences set forth in SEQ ID NOs: 11, 31, 50 respectively.

3. The antibody or antigen-binding fragment thereof according to claim 1, wherein
(1) the VL comprises the amino acid sequence having at least 80%, at least 85%, at least 90%, at least 95%, at least 98%, at least 99%, or 100% sequence identity to SEQ ID No: 136, and the VH comprises the amino acid sequence having at least 80%, at least 85%, at least 90%, at least 95%, at least 98%, at least 99%, or 100% sequence identity to SEQ ID No: 117; or
(2) the VL comprises the amino acid sequence having at least 80%, at least 85%, at least 90%, at least 95%, at least 98%, at least 99%, or 100% sequence identity to SEQ ID No: 124, and the VH comprises the amino acid sequence having at least 80%, at least 85%, at least 90%, at least 95%, at least 98%, at least 99%, or 100% sequence identity to SEQ ID No: 111; or
(3) the VL comprises the amino acid sequence having at least 80%, at least 85%, at least 90%, at least 95%, at least 98%, at least 99%, or 100% sequence identity to SEQ ID No: 133, and the VH comprises the amino acid sequence having at least 80%, at least 85%, at least 90%, at least 95%, at least 98%, at least 99%, or 100% sequence identity to SEQ ID No: 117; or
(4) the VL comprises the amino acid sequence having at least 80%, at least 85%, at least 90%, at least 95%, at least 98%, at least 99%, or 100% sequence identity to SEQ ID No: 134, and the VH comprises the amino acid sequence having at least 80%, at least 85%, at least 90%, at least 95%, at least 98%, at least 99%, or 100% sequence identity to SEQ ID No: 118; or
(5) the VL comprises the amino acid sequence having at least 80%, at least 85%, at least 90%, at least 95%, at least 98%, at least 99%, or 100% sequence identity to SEQ ID No: 135, and the VH comprises the amino acid sequence having at least 80%, at least 85%, at least 90%, at least 95%, at least 98%, at least 99%, or 100% sequence identity to SEQ ID No: 119; or
(6) the VL comprises the amino acid sequence having at least 80%, at least 85%, at least 90%, at least 95%, at least 98%, at least 99%, or 100% sequence identity to SEQ ID No: 137, and the VH comprises the amino acid sequence having at least 80%, at least 85%, at least 90%, at least 95%, at least 98%, at least 99%, or 100% sequence identity to SEQ ID No: 120; or
(7) the VL comprises the amino acid sequence having at least 80%, at least 85%, at least 90%, at least 95%, at least 98%, at least 99%, or 100% sequence identity to SEQ ID No: 123, and the VH comprises the amino acid sequence having at least 80%, at least 85%, at least 90%, at least 95%, at least 98%, at least 99%, or 100% sequence identity to SEQ ID No: 108; or
(8) the VL comprises the amino acid sequence having at least 80%, at least 85%, at least 90%, at least 95%, at least 98%, at least 99%, or 100% sequence identity to SEQ ID No: 124, and the VH comprises the amino acid sequence having at least 80%, at least 85%, at least 90%, at least 95%, at least 98%, at least 99%, or 100% sequence identity to SEQ ID No: 109; or
(9) the VL comprises the amino acid sequence having at least 80%, at least 85%, at least 90%, at least 95%, at least 98%, at least 99%, or 100% sequence identity to SEQ ID No: 125, and the VH comprises the amino acid sequence having at least 80%, at least 85%, at least 90%, at least 95%, at least 98%, at least 99%, or 100% sequence identity to SEQ ID No: 110; or
(10) the VL comprises the amino acid sequence having at least 80%, at least 85%, at least 90%, at least 95%, at least 98%, at least 99%, or 100% sequence identity to SEQ ID No: 128, and the VH comprises the amino acid sequence having at least 80%, at least 85%, at least 90%, at least 95%, at least 98%, at least 99%, or 100% sequence identity to SEQ ID No: 114; or
(11) the VL comprises the amino acid sequence having at least 80%, at least 85%, at least 90%, at least 95%, at least 98%, at least 99%, or 100% sequence identity to SEQ ID No: 129, and the VH comprises the amino acid sequence having at least 80%, at least 85%, at least 90%, at least 95%, at least 98%, at least 99%, or 100% sequence identity to SEQ ID No:115; or
(12) the VL comprises the amino acid sequence having at least 80%, at least 85%, at least 90%, at least 95%, at least 98%, at least 99%, or 100% sequence identity to SEQ ID No: 130, and the VH comprises the amino acid sequence having at least 80%, at least 85%, at least 90%, at least 95%, at least 98%, at least 99%, or 100% sequence identity to SEQ ID No: 116; or
(13) the VL comprises the amino acid sequence having at least 80%, at least 85%, at least 90%, at least 95%, at least 98%, at least 99%, or 100% sequence identity to SEQ ID No: 131, and the VH comprises the amino acid sequence having at least 80%, at least 85%, at least 90%, at least 95%, at least 98%, at least 99%, or 100% sequence identity to SEQ ID No: 115; or
(14) the VL comprises the amino acid sequence having at least 80%, at least 85%, at least 90%, at least 95%, at least 98%, at least 99%, or 100% sequence identity to SEQ ID No: 132, and the VH comprises the amino acid sequence having at least 80%, at least 85%, at least 90%, at least 95%, at least 98%, at least 99%, or 100% sequence identity to SEQ ID No: 116.

4. The antibody or antigen-binding fragment thereof according to claim 1, wherein the antibody or antigen-binding fragment comprises a heavy chain (HC) and a light chain (LC), and wherein
(1) the LC comprises the amino acid sequence having at least 80%, at least 85%, at least 90%, at least 95%, at least 98%, at least 99%, or 100% sequence identity to SEQ ID No: 170, and the HC comprises the amino acid sequence having at least 80%, at least 85%, at least 90%, at least 95%, at least 98%, at least 99%, or 100% sequence identity to SEQ ID No: 151; or
(2) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 143; or
(3) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 147; or
(4) the LC comprises the amino acid sequence having at least 80%, at least 85%, at least 90%, at least 95%, at least 98%, at least 99%, or 100% sequence identity to SEQ ID No: 167, and the HC comprises the amino acid sequence having at least 80%, at least 85%, at least 90%, at least 95%, at least 98%, at least 99%, or 100% sequence identity to SEQ ID No: 151; or
(5) the LC comprises the amino acid sequence having at least 80%, at least 85%, at least 90%, at least 95%, at least 98%, at least 99%, or 100% sequence identity to SEQ ID No: 168, and the HC comprises the amino acid sequence having at least 80%, at least 85%, at least 90%, at least 95%, at least 98%, at least 99%, or 100% sequence identity to SEQ ID No: 152; or
(6) the LC comprises the amino acid sequence having at least 80%, at least 85%, at least 90%, at least 95%, at least 98%, at least 99%, or 100% sequence identity to SEQ ID No: 169, and the HC comprises the amino acid sequence having at least 80%, at least 85%, at least 90%, at least 95%, at least 98%, at least 99%, or 100% sequence identity to SEQ ID No: 153; or
(7) the LC comprises the amino acid sequence having at least 80%, at least 85%, at least 90%, at least 95%, at least 98%, at least 99%, or 100% sequence identity to SEQ ID No: 171, and the HC comprises the amino acid sequence having at least 80%, at least 85%, at least 90%, at least 95%, at least 98%, at least 99%, or 100% sequence identity to SEQ ID No: 154; or
(8) the LC comprises the amino acid sequence having at least 80%, at least 85%, at least 90%, at least 95%, at least 98%, at least 99%, or 100% sequence identity to SEQ ID No: 157, and the HC comprises the amino acid sequence having at least 80%, at least 85%, at least 90%, at least 95%, at least 98%, at least 99%, or 100% sequence identity to SEQ ID No: 140; or
(9) the LC comprises the amino acid sequence having at least 80%, at least 85%, at least 90%, at least 95%, at least 98%, at least 99%, or 100% sequence identity to SEQ ID No: 158, and the HC comprises the amino acid sequence having at least 80%, at least 85%, at least 90%, at least 95%, at least 98%, at least 99%, or 100% sequence identity to SEQ ID No: 141; or
(10) the LC comprises the amino acid sequence having at least 80%, at least 85%, at least 90%, at least 95%, at least 98%, at least 99%, or 100% sequence identity to SEQ ID No: 159, and the HC comprises the amino acid sequence having at least 80%, at least 85%, at least 90%, at least 95%, at least 98%, at least 99%, or 100% sequence identity to SEQ ID No: 142; or
(11) the LC comprises the amino acid sequence having at least 80%, at least 85%, at least 90%, at least 95%, at least 98%, at least 99%, or 100% sequence identity to SEQ ID No: 162, and the HC comprises the amino acid sequence having at least 80%, at least 85%, at least 90%, at least 95%, at least 98%, at least 99%, or 100% sequence identity to SEQ ID No: 146; or
(12) the LC comprises the amino acid sequence having at least 80%, at least 85%, at least 90%, at least 95%, at least 98%, at least 99%, or 100% sequence identity to SEQ ID No: 162, and the HC comprises the amino acid sequence having at least 80%, at least 85%, at least 90%, at least 95%, at least 98%, at least 99%, or 100% sequence identity to SEQ ID No: 148; or
(13) the LC comprises the amino acid sequence having at least 80%, at least 85%, at least 90%, at least 95%, at least 98%, at least 99%, or 100% sequence identity to SEQ ID No: 163, and the HC comprises the amino acid sequence having at least 80%, at least 85%, at least 90%, at least 95%, at least 98%, at least 99%, or 100% sequence identity to SEQ ID No: 149; or
(14) the LC comprises the amino acid sequence having at least 80%, at least 85%, at least 90%, at least 95%, at least 98%, at least 99%, or 100% sequence identity to SEQ ID No: 164, and the HC comprises the amino acid sequence having at least 80%, at least 85%, at least 90%, at least 95%, at least 98%, at least 99%, or 100% sequence identity to SEQ ID No: 150; or
(15) the LC comprises the amino acid sequence having at least 80%, at least 85%, at least 90%, at least 95%, at least 98%, at least 99%, or 100% sequence identity to SEQ ID No: 165, and the HC comprises the amino acid sequence having at least 80%, at least 85%, at least 90%, at least 95%, at least 98%, at least 99%, or 100% sequence identity to SEQ ID No: 149; or
(16) the LC comprises the amino acid sequence having at least 80%, at least 85%, at least 90%, at least 95%, at least 98%, at least 99%, or 100% sequence identity to SEQ ID No: 166, and the HC comprises the amino acid sequence having at least 80%, at least 85%, at least 90%, at least 95%, at least 98%, at least 99%, or 100% sequence identity to SEQ ID No: 150.

5. The antibody or antigen-binding fragment thereof according to claim 1, wherein the antibody is a murine antibody, a chimeric antibody, a humanized antibody, or a human antibody.

6. The antibody or antigen-binding fragment thereof according to claim 1, wherein the antibody is of an isotype selected from the group consisting of IgG, IgA, IgM, IgE and IgD; and/or
wherein the antigen-binding fragment is selected from the group consisting of Fab, Fab′, F(ab′)₂, Fd, Fd′, Fv, scFv, ds-scFv, dAb, and sdAb.

7. The antibody or the antigen binding fragment thereof according to claim 1, wherein the antibody is of a subtype selected from the group consisting of IgG1, IgG2, IgG3, and IgG4.

8. (canceled)

9. The antibody or antigen-binding fragment thereof according to claim 1, wherein the antibody is a monoclonal antibody, a bi-specific antibody, or a multi-specific antibody.

10. The antibody or antigen-binding fragment thereof according to claim 1, wherein the antibody is attached to a fluorescent label, radiolabel or cytotoxic agent.

11. A bi-specific antibody, comprising the antibody or antigen-binding fragment thereof according to claim 1 and a second antigen binding region specifically binding to a tumor associated antigen or an immune checkpoint molecule.

12. A nucleic acid comprising a nucleotide sequence encoding the antibody or the antigen-binding fragment thereof according to claim 1 or a bi-specific antibody comprising the antibody or the antigen-binding fragment thereof and a second antigen binding region specifically binding to a tumor associated antigen or an immune checkpoint molecule.

13. A vector comprising the nucleic acid according to claim 12.

14. A host cell comprising the nucleic acid according to claim 12 or a vector comprising the nucleic acid according to claim 12.

15. An antibody-drug conjugate (ADC), comprising the antibody or the antigen-binding fragment thereof according to claim 1 or a bi-specific antibody comprising the antibody or antigen-binding fragment thereof according to claim 1 and a second antigen binding region specifically binding to a tumor associated antigen or an immune checkpoint molecule.

16. A pharmaceutical composition comprising the antibody or the antigen-binding fragment thereof according to claim 1, a bi-specific antibody comprising the antibody or the antigen-binding fragment thereof and a second antigen binding region specifically binding to a tumor associated antigen or an immune checkpoint molecule or an antibody-drug conjugate comprising the antibody or the antigen-binding fragment thereof or the bispecific antibody, and a pharmaceutically acceptable carrier or excipient.

17. The pharmaceutical composition according to claim 16, wherein the composition further comprises a second therapeutic agent selected from the group consisting of an antibody, a chemotherapeutic agent, an siRNA, antisense oligonucleotide, a polypeptide, and a small molecule drug.

18. A method of treating a cancer in a subject, comprising administering to the subject an effective amount of the antibody or the antigen-binding fragment thereof according to claim 1, a bi-specific antibody comprising the antibody or the antigen-binding fragment thereof and a second antigen binding region specifically binding to a tumor associated antigen or an immune checkpoint molecule, or an antibody-drug conjugate comprising the antibody or the antigen-binding fragment thereof or the bispecific antibody.

19. A method of activating T cells and/or myeloid cells in a cancer microenvironment in a subject, comprising administering to the subject an effective amount of the antibody or the antigen-binding fragment thereof according to claim 1, a bi-specific antibody comprising the antibody or the antigen-binding fragment thereof and a second antigen binding region specifically binding to a tumor associated antigen or an immune checkpoint molecule, an antibody-drug conjugate comprising the antibody or the antigen-binding fragment thereof or the bispecific antibody.

20. The method according to claim 18, wherein the cancer is selected from the group consisting of pancreatic ductal adenocarcinoma, prostate cancer, melanoma, liver cancer, breast cancer, lung cancer, lung squamous cell carcinoma), squamous cell carcinoma, ovarian cancer, leukemia, and myeloma.

21. The method according to claim 18, further comprising administering to the subject a second therapeutic agent, the second therapeutic agent is selected from an antibody, a chemotherapeutic agent, a siRNA, an antisense oligonucleotide, a polypeptide, and a small molecule drug.

22-31. (canceled)


SEQUENCE LISTING (185 sequences)

SEQ ID NO: 1 - protein, 25 aa, synthetic construct
  REGION 1..25: note = PR002903 HFWR1 Chothia
QVQLQQSGSELKKPGASVKISCKAS

SEQ ID NO: 2 - protein, 25 aa, synthetic construct
  REGION 1..25: note = PR002906 HFWR1 Chothia
QVQLQESGPGLVKPSETLSLTCTVS

SEQ ID NO: 3 - protein, 25 aa, synthetic construct
  REGION 1..25: note = PR005474 HFWR1 Chothia, PR005509 HFWR1 Chothia, PR005514 HFWR1 Chothia, PR006839 HFWR1 Chothia, PR007630 HFWR1 Chothia, PR007631 HFWR1 Chothia, PR007972 HFWR1 Chothia, PR007973 HFWR1 Chothia, PR007974
QVQLVESGGGVVQPGRSLRLSCAAS

SEQ ID NO: 4 - protein, 25 aa, synthetic construct
  REGION 1..25: note = PR005512 HFWR1 Chothia
QVQLVESGGGVVQPGRSLRLSCVAS

SEQ ID NO: 5 - protein, 25 aa, synthetic construct [sequence omitted]

SEQ ID NO: 6 - protein, 25 aa, synthetic construct
  REGION 1..25: note = PR006475 HFWR1 Chothia
QVQLQESGPGLVKPSGTLSLTCAVS

SEQ ID NO: 7 - protein, 25 aa, synthetic construct
  REGION 1..25: note = PR006480 HFWR1 Chothia, PR006895 HFWR1 Chothia, PR007221 HFWR1 Chothia, PR007223 HFWR1 Chothia, PR007442 HFWR1 Chothia, PR007444 HFWR1 Chothia
QVQLVQSGGGVVQPGRSLRLSCAAS

SEQ ID NO: 8 - protein, 7 aa, synthetic construct
  REGION 1..7: note = PR002903 HCDR1 Chothia
GYSFTDY

SEQ ID NO: 9 - protein, 8 aa, synthetic construct
  REGION 1..8: note = PR002906 HCDR1 Chothia
GYTITSGY

SEQ ID NO: 10 - protein, 7 aa, synthetic construct
  REGION 1..7: note = PR005474 HCDR1 Chothia
AFTFSSY

SEQ ID NO: 11 - protein, 7 aa, synthetic construct
  REGION 1..7: note = PR005509 HCDR1 Chothia, PR005512 HCDR1 Chothia, PR005514 HCDR1 Chothia, PR006480 HCDR1 Chothia, PR006839 HCDR1 Chothia, PR006895 HCDR1 Chothia, PR007221 HCDR1 Chothia, PR007223 HCDR1 Chothia, PR007442
GFTFSSY

SEQ ID NO: 12 - protein, 8 aa, synthetic construct
  REGION 1..8: note = PR006207 HCDR1 Chothia
GFSFSSGY

SEQ ID NO: 13 - protein, 8 aa, synthetic construct
  REGION 1..8: note = PR006475 HCDR1 Chothia
GASISSGH

SEQ ID NO: 14 - protein, 7 aa, synthetic construct
  REGION 1..7: note = PR007972 HCDR1 Chothia, PR007974 HCDR1 Chothia
GFTFSYY

SEQ ID NO: 15 - protein, 19 aa, synthetic construct
  REGION 1..19: note = PR002903 HFWR2 Chothia
IILWVRQNPGKGLEWIGHI

SEQ ID NO: 16 - protein, 19 aa, synthetic construct
  REGION 1..19: note = PR002906 HFWR2 Chothia
DWSWIRQPPGKGLEWIGYI

SEQ ID NO: 17 - protein, 19 aa, synthetic construct
  REGION 1..19: note = PR005474 HFWR2 Chothia, PR006480 HFWR2 Chothia, PR006895 HFWR2 Chothia, PR007221 HFWR2 Chothia, PR007223 HFWR2 Chothia, PR007442 HFWR2 Chothia, PR007444 HFWR2 Chothia
GMHWVRQAPGKGLEWVALI

SEQ ID NO: 18 - protein, 19 aa, synthetic construct
  REGION 1..19: note = PR005509 HFWR2 Chothia, PR005514 HFWR2 Chothia, PR006839 HFWR2 Chothia, PR007631 HFWR2 Chothia, PR007974 HFWR2 Chothia
GFHWVRQAPGKGLEWLGLI

SEQ ID NO: 19 - protein, 19 aa, synthetic construct
  REGION 1..19: note = PR005512 HFWR2 Chothia
GMHWVRQAPGKGLEWVAII

SEQ ID NO: 20 - protein, 19 aa, synthetic construct
  REGION 1..19: note = PR006207 HFWR2 Chothia
YMAWVRQAPGQGLEWMGLI

SEQ ID NO: 21 - protein, 19 aa, synthetic construct
  REGION 1..19: note = PR006475 HFWR2 Chothia
WWSWVRQPPGKGLEWIGEI

SEQ ID NO: 22 - protein, 19 aa, synthetic construct
  REGION 1..19: note = PR007630 HFWR2 Chothia, PR007972 HFWR2 Chothia, PR007973 HFWR2 Chothia
GFHWVRQAPGKGLEWLGII

SEQ ID NO: 23 - protein, 6 aa, synthetic construct
  REGION 1..6: note = PR002903 HCDR2 Chothia
DPYYGS

SEQ ID NO: 24 - protein, 5 aa, synthetic construct
  REGION 1..5: note = PR002906 HCDR2 Chothia
NYGGS

SEQ ID NO: 25 - protein, 6 aa, synthetic construct
  REGION 1..6: note = PR005474 HCDR2 Chothia, PR005512 HCDR2 Chothia
WNDGSN

SEQ ID NO: 26 - protein, 6 aa, synthetic construct
  REGION 1..6: note = PR005509 HCDR2 Chothia, PR005514 HCDR2 Chothia, PR006839 HCDR2 Chothia
WSDGSN

SEQ ID NO: 27 - protein, 6 aa, synthetic construct
  REGION 1..6: note = PR006207 HCDR2 Chothia
GVGSGS

SEQ ID NO: 28 - protein, 5 aa, synthetic construct
  REGION 1..5: note = PR006475 HCDR2 Chothia
YHSGS

SEQ ID NO: 29 - protein, 6 aa, synthetic construct
  REGION 1..6: note = PR006480 HCDR2 Chothia, PR006895 HCDR2 Chothia
WYDGSN

SEQ ID NO: 30 - protein, 6 aa, synthetic construct
  REGION 1..6: note = PR007221 HCDR2 Chothia, PR007442 HCDR2 Chothia
WYDASY

SEQ ID NO: 31 - protein, 6 aa, synthetic construct
  REGION 1..6: note = PR007223 HCDR2 Chothia, PR007444 HCDR2 Chothia
WYDASN

SEQ ID NO: 32 - protein, 6 aa, synthetic construct
  REGION 1..6: note = PR007630 HCDR2 Chothia, PR007972 HCDR2 Chothia, PR007973 HCDR2 Chothia
WSDASN

SEQ ID NO: 33 - protein, 6 aa, synthetic construct
  REGION 1..6: note = PR007631 HCDR2 Chothia, PR007974 HCDR2 Chothia
WSDARH

SEQ ID NO: 34 - protein, 41 aa, synthetic construct
  REGION 1..41: note = PR002903 HFWR3 Chothia
SNYNLKFKGRVTITADQSTTTAYMELSSLRSEDTAVYYCGR

SEQ ID NO: 35 - protein, 41 aa, synthetic construct
  REGION 1..41: note = PR002906 HFWR3 Chothia
TNYKPSLGSRVTISVDTSKNQFSLKLSSVTAADTAVYYCAR

SEQ ID NO: 36 - protein, 41 aa, synthetic construct
  REGION 1..41: note = PR005474 HFWR3 Chothia
KFYADSVKGRFTISRDNSKNTLFLQMNSLRAEDTAVYYCAR

SEQ ID NO: 37 - protein, 41 aa, synthetic construct
  REGION 1..41: note = PR005509 HFWR3 Chothia, PR005514 HFWR3 Chothia, PR006480 HFWR3 Chothia, PR006839 HFWR3 Chothia, PR006895 HFWR3 Chothia, PR007221 HFWR3 Chothia, PR007223 HFWR3 Chothia, PR007442 HFWR3 Chothia, PR007444
KYYADSVKGRFTISRDNSKNTLYLQMNSLRAEDTAVYYCAR

SEQ ID NO: 38 - protein, 41 aa, synthetic construct
  REGION 1..41: note = PR005512 HFWR3 Chothia
RYYADSVKGRFTISRDNSKNTLYLQMNSLRAEDTAVYYCAR

SEQ ID NO: 39 - protein, 41 aa, synthetic construct
  REGION 1..41: note = PR006207 HFWR3 Chothia
LWYAQKFQGRVTMTRDTSTSTVYMELSSLRSEDTAVYYCAR

SEQ ID NO: 40 - protein, 41 aa, synthetic construct
  REGION 1..41: note = PR006475 HFWR3 Chothia
TNYTPSLKSRVTISVDKSKNQFSLKLTSVTAADTAVYYCAR

SEQ ID NO: 41 - protein, 41 aa, synthetic construct
  REGION 1..41: note = PR007631 HFWR3 Chothia, PR007974 HFWR3 Chothia
KDYADSVKGRFTISRDNSKNTLYLQMNSLRAEDTAVYYCAR

SEQ ID NO: 42 - protein, 8 aa, synthetic construct
  REGION 1..8: note = PR002903 HCDR3 Chothia
SKRDYFDY

SEQ ID NO: 43 - protein, 15 aa, synthetic construct
  REGION 1..15: note = PR002906 HCDR3 Chothia
YNEYKSYIYDWYFDF

SEQ ID NO: 44 - protein, 10 aa, synthetic construct
  REGION 1..10: note = PR005474 HCDR3 Chothia
DRILGAGFDY

SEQ ID NO: 45 - protein, 10 aa, synthetic construct
  REGION 1..10: note = PR005509 HCDR3 Chothia, PR005512 HCDR3 Chothia
DRIVGAGFDY

SEQ ID NO: 46 - protein, 10 aa, synthetic construct
  REGION 1..10: note = PR005514 HCDR3 Chothia, PR006839 HCDR3 Chothia, PR007630 HCDR3 Chothia, PR007631 HCDR3 Chothia, PR007972 HCDR3 Chothia, PR007973 HCDR3 Chothia, PR007974 HCDR3 Chothia
DRIVGEGFDY

SEQ ID NO: 47 - protein, 10 aa, synthetic construct
  REGION 1..10: note = PR006207 HCDR3 Chothia
HFALSDPFNL

SEQ ID NO: 48 - protein, 17 aa, synthetic construct
  REGION 1..17: note = PR006475 HCDR3 Chothia
EASYHYGSGSYYTPFDF

SEQ ID NO: 49 - protein, 7 aa, synthetic construct
  REGION 1..7: note = PR006480 HCDR3 Chothia, PR006895 HCDR3 Chothia, PR007221 HCDR3 Chothia, PR007442 HCDR3 Chothia
SVLLFDS

SEQ ID NO: 50 - protein, 7 aa, synthetic construct
  REGION 1..7: note = PR007223 HCDR3 Chothia, PR007444 HCDR3 Chothia
SVLLFNM

SEQ ID NO: 51 - protein, 11 aa, synthetic construct
  REGION 1..11: note = PR002903 HFWR4 Chothia
WGQGTTLTVSS

SEQ ID NO: 52 - protein, 11 aa, synthetic construct
  REGION 1..11: note = PR002906 HFWR4 Chothia, PR005474 HFWR4 Chothia, PR005509 HFWR4 Chothia, PR005512 HFWR4 Chothia, PR005514 HFWR4 Chothia, PR006207 HFWR4 Chothia, PR006475 HFWR4 Chothia, PR006480 HFWR4 Chothia, PR006839
WGQGTLVTVSS

SEQ ID NO: 53 - protein, 23 aa, synthetic construct
  REGION 1..23: note = PR002903 LFWR1 Chothia
DIQMTQSPSSLSASIGDRVTITC

SEQ ID NO: 54 - protein, 23 aa, synthetic construct
  REGION 1..23: note = PR002906 LFWR1 Chothia
DIQMTQSPSSLSASVGDRVTITC

SEQ ID NO: 55 - protein, 23 aa, synthetic construct
  REGION 1..23: note = PR005474 LFWR1 Chothia, PR005509 LFWR1 Chothia, PR005512 LFWR1 Chothia, PR005514 LFWR1 Chothia, PR006839 LFWR1 Chothia, PR007630 LFWR1 Chothia, PR007631 LFWR1 Chothia, PR007972 LFWR1 Chothia, PR007973
DIVMTQSPLSLPVTPGEPASISC

SEQ ID NO: 56 - protein, 23 aa, synthetic construct
  REGION 1..23: note = PR006207 LFWR1 Chothia
EIVLTQSPDFQSVTPKEKVTITC

SEQ ID NO: 57 - protein, 23 aa, synthetic construct
  REGION 1..23: note = PR006475 LFWR1 Chothia
EIVLTQSPATLSVSPGERATLSC

SEQ ID NO: 58 - protein, 23 aa, synthetic construct
  REGION 1..23: note = PR006480 LFWR1 Chothia, PR006895 LFWR1 Chothia, PR007442 LFWR1 Chothia, PR007444 LFWR1 Chothia
DIQMTQSPPSLSASVGDRVTITC

SEQ ID NO: 59 - protein, 23 aa, synthetic construct
  REGION 1..23: note = PR007221 LFWR1 Chothia, PR007223 LFWR1 Chothia
DIQLTQSPPSLSASVGDRVTITC

SEQ ID NO: 60 - protein, 11 aa, synthetic construct
  REGION 1..11: note = PR002903 LCDR1 Chothia
KASQDINSYLS

SEQ ID NO: 61 - protein, 11 aa, synthetic construct
  REGION 1..11: note = PR002906 LCDR1 Chothia
KASKNIRSYLA

SEQ ID NO: 62 - protein, 16 aa, synthetic construct
  REGION 1..16: note = PR005474 LCDR1 Chothia, PR005509 LCDR1 Chothia, PR005512 LCDR1 Chothia, PR005514 LCDR1 Chothia, PR006839 LCDR1 Chothia
RSSQSLLHSNGYNYLD

SEQ ID NO: 63 - protein, 11 aa, synthetic construct
  REGION 1..11: note = PR006207 LCDR1 Chothia
QASESIDSYLL

SEQ ID NO: 64 - protein, 11 aa, synthetic construct
  REGION 1..11: note = PR006475 LCDR1 Chothia
RASQSVSSNLA

SEQ ID NO: 65 - protein, 11 aa, synthetic construct
  REGION 1..11: note = PR006480 LCDR1 Chothia, PR006895 LCDR1 Chothia, PR007221 LCDR1 Chothia, PR007223 LCDR1 Chothia, PR007442 LCDR1 Chothia, PR007444 LCDR1 Chothia
RASQSISSYLN

SEQ ID NO: 66 - protein, 16 aa, synthetic construct
  REGION 1..16: note = PR007630 LCDR1 Chothia, PR007631 LCDR1 Chothia, PR007974 LCDR1 Chothia
RSSQSLLHRSGYNYLD

SEQ ID NO: 67 - protein, 16 aa, synthetic construct
  REGION 1..16: note = PR007972 LCDR1 Chothia, PR007973 LCDR1 Chothia
RSSQSLLHRNGYNYLD

SEQ ID NO: 68 - protein, 15 aa, synthetic construct
  REGION 1..15: note = PR002903 LFWR2 Chothia
WFQQKPGKAPKLLIY

SEQ ID NO: 69 - protein, 15 aa, synthetic construct
  REGION 1..15: note = PR002906 LFWR2 Chothia
WYQQKPGKAPKLLIY

SEQ ID NO: 70 - protein, 15 aa, synthetic construct
  REGION 1..15: note = PR005474 LFWR2 Chothia, PR005509 LFWR2 Chothia, PR005512 LFWR2 Chothia, PR005514 LFWR2 Chothia, PR006839 LFWR2 Chothia, PR007630 LFWR2 Chothia, PR007631 LFWR2 Chothia, PR007972 LFWR2 Chothia, PR007973
WYLQKPGQSPQLLIY

SEQ ID NO: 71 - protein, 15 aa, synthetic construct
  REGION 1..15: note = PR006207 LFWR2 Chothia
WYQQKPDQSPKLLIK

SEQ ID NO: 72 - protein, 15 aa, synthetic construct
  REGION 1..15: note = PR006475 LFWR2 Chothia
WYQQKPGQAPRLLIY

SEQ ID NO: 73 - protein, 15 aa, synthetic construct
  REGION 1..15: note = PR006480 LFWR2 Chothia, PR006895 LFWR2 Chothia, PR007221 LFWR2 Chothia, PR007223 LFWR2 Chothia, PR007442 LFWR2 Chothia, PR007444 LFWR2 Chothia
WFQQKAGKAPKLLIS

SEQ ID NO: 74 - protein, 7 aa, synthetic construct
  REGION 1..7: note = PR002903 LCDR2 Chothia
RANRLVD

SEQ ID NO: 75 - protein, 7 aa, synthetic construct
  REGION 1..7: note = PR002906 LCDR2 Chothia
SGSTLHS

SEQ ID NO: 76 - protein, 7 aa, synthetic construct
  REGION 1..7: note = PR005474 LCDR2 Chothia, PR005509 LCDR2 Chothia, PR005512 LCDR2 Chothia, PR005514 LCDR2 Chothia, PR006839 LCDR2 Chothia, PR007972 LCDR2 Chothia
LGSNRAS

SEQ ID NO: 77 - protein, 7 aa, synthetic construct
  REGION 1..7: note = PR006207 LCDR2 Chothia
QASTLAS

SEQ ID NO: 78 - protein, 7 aa, synthetic construct
  REGION 1..7: note = PR006475 LCDR2 Chothia
GASTRAT

SEQ ID NO: 79 - protein, 7 aa, synthetic construct
  REGION 1..7: note = PR006480 LCDR2 Chothia, PR006895 LCDR2 Chothia, PR007221 LCDR2 Chothia, PR007223 LCDR2 Chothia, PR007442 LCDR2 Chothia, PR007444 LCDR2 Chothia
ATSNLQS

SEQ ID NO: 80 - protein, 7 aa, synthetic construct
  REGION 1..7: note = PR007630 LCDR2 Chothia, PR007973 LCDR2 Chothia, PR007974 LCDR2 Chothia
LGSYRAS

SEQ ID NO: 81 - protein, 7 aa, synthetic construct
  REGION 1..7: note = PR007631 LCDR2 Chothia
LGSYRWS

SEQ ID NO: 82 - protein, 32 aa, synthetic construct
  REGION 1..32: note = PR002903 LFWR3 Chothia
GVPSRFSGSGSGTDYTLTISSLQPEDFAVYYC

SEQ ID NO: 83 - protein, 32 aa, synthetic construct
  REGION 1..32: note = PR002906 LFWR3 Chothia
GVPSRFSGSGSGTDFTLTISSLQPEDFATYYC

SEQ ID NO: 84 - protein, 32 aa, synthetic construct
  REGION 1..32: note = PR005474 LFWR3 Chothia, PR005509 LFWR3 Chothia, PR005514 LFWR3 Chothia, PR006839 LFWR3 Chothia, PR007630 LFWR3 Chothia, PR007631 LFWR3 Chothia, PR007972 LFWR3 Chothia, PR007973 LFWR3 Chothia, PR007974
GVPDRFSGSGSGTDFTLKISRVEAEDVGVYYC

SEQ ID NO: 85 - protein, 32 aa, synthetic construct
  REGION 1..32: note = PR005512 LFWR3 Chothia
GVPDRFSGSGSGTDFTLKISRLEAEDVGVYYC

SEQ ID NO: 86 - protein, 32 aa, synthetic construct
  REGION 1..32: note = PR006207 LFWR3 Chothia
GVPSRFSGSGSGTDFTLTINSLEAEDAATYYC

SEQ ID NO: 87 - protein, 32 aa, synthetic construct
  REGION 1..32: note = PR006475 LFWR3 Chothia
GIPARFSGSGSGTEFTLTISSLQSEDFALYYC

SEQ ID NO: 88 - protein, 32 aa, synthetic construct
  REGION 1..32: note = PR006480 LFWR3 Chothia, PR006895 LFWR3 Chothia, PR007221 LFWR3 Chothia, PR007223 LFWR3 Chothia, PR007442 LFWR3 Chothia, PR007444 LFWR3 Chothia
GVPSRFSGSGSGTDFTLTISSLQPEDLATYYC

SEQ ID NO: 89 - protein, 9 aa, synthetic construct
  REGION 1..9: note = PR002903 LCDR3 Chothia
LQYDEFPYT

SEQ ID NO: 90 - protein, 9 aa, synthetic construct
  REGION 1..9: note = PR002906 LCDR3 Chothia
QQHHEYPLT

SEQ ID NO: 91 - protein, 9 aa, synthetic construct
  REGION 1..9: note = PR005474 LCDR3 Chothia
MQALQTPWT

SEQ ID NO: 92 - protein, 9 aa, synthetic construct
  REGION 1..9: note = PR005509 LCDR3 Chothia, PR005514 LCDR3 Chothia, PR006839 LCDR3 Chothia, PR007630 LCDR3 Chothia, PR007631 LCDR3 Chothia
MQGLQTMYT

SEQ ID NO: 93 - protein, 9 aa, synthetic construct
  REGION 1..9: note = PR005512 LCDR3 Chothia
MQGLQTPWT

SEQ ID NO: 94 - protein, 10 aa, synthetic construct
  REGION 1..10: note = PR006207 LCDR3 Chothia
QNYYDISSND

SEQ ID NO: 95 - protein, 8 aa, synthetic construct
  REGION 1..8: note = PR006475 LCDR3 Chothia
QQYNNWII

SEQ ID NO: 96 - protein, 9 aa, synthetic construct
  REGION 1..9: note = PR006480 LCDR3 Chothia, PR006895 LCDR3 Chothia
QQSYSSPYT

SEQ ID NO: 97 - protein, 9 aa, synthetic construct
  REGION 1..9: note = PR007221 LCDR3 Chothia, PR007442 LCDR3 Chothia
QQTLSSPYT

SEQ ID NO: 98 - protein, 9 aa, synthetic construct
  REGION 1..9: note = PR007223 LCDR3 Chothia, PR007444 LCDR3 Chothia
QQTYSSPYT

SEQ ID NO: 99 - protein, 9 aa, synthetic construct
  REGION 1..9: note = PR007972 LCDR3 Chothia, PR007973 LCDR3 Chothia
QQGIQTEYT

SEQ ID NO: 100 - protein, 9 aa, synthetic construct
  REGION 1..9: note = PR007974 LCDR3 Chothia
QQGIQTTYT

SEQ ID NO: 101 - protein, 10 aa, synthetic construct
  REGION 1..10: note = PR002903 LFWR4 Chothia
FGGGTKLEIK

SEQ ID NO: 102 - protein, 10 aa, synthetic construct
  REGION 1..10: note = PR002906 LFWR4 Chothia, PR005474 LFWR4 Chothia, PR005512 LFWR4 Chothia, PR006480 LFWR4 Chothia, PR006895 LFWR4 Chothia, PR007221 LFWR4 Chothia, PR007223 LFWR4 Chothia, PR007442 LFWR4 Chothia, PR007444
FGQGTKVEIK

SEQ ID NO: 103 - protein, 10 aa, synthetic construct
  REGION 1..10: note = PR005509 LFWR4 Chothia, PR005514 LFWR4 Chothia, PR006839 LFWR4 Chothia, PR007630 LFWR4 Chothia, PR007631 LFWR4 Chothia, PR007972 LFWR4 Chothia, PR007973 LFWR4 Chothia, PR007974 LFWR4 Chothia
FGQGTKLEIK

SEQ ID NO: 104 - protein, 10 aa, synthetic construct
  REGION 1..10: note = PR006207 LFWR4 Chothia
FGGGTKVEIK

SEQ ID NO: 105 - protein, 10 aa, synthetic construct
  REGION 1..10: note = PR006475 LFWR4 Chothia
FGQGTRLEIK

SEQ ID NO: 106 - protein, 117 aa, synthetic construct
  REGION 1..117: note = PR002903 VH
QVQLQQSGSELKKPGASVKISCKASGYSFTDYIILWVRQNPGKGLEWIGHIDPYYGSSNYNLKFKGRVTITADQSTTTAYMELSSLRSEDTAVYYCGRSKRDYFDYWGQGTTLTVSS

SEQ ID NO: 107 - protein, 124 aa, synthetic construct
  REGION 1..124: note = PR002906 VH
QVQLQESGPGLVKPSETLSLTCTVSGYTITSGYDWSWIRQPPGKGLEWIGYINYGGSTNYKPSLGSRVTISVDTSKNQFSLKLSSVTAADTAVYYCARYNEYKSYIYDWYFDFWGQGTLVTVSS

SEQ ID NO: 108 - protein, 119 aa, synthetic construct
  REGION 1..119: note = PR005474 VH
QVQLVESGGGVVQPGRSLRLSCAASAFTFSSYGMHWVRQAPGKGLEWVALIWNDGSNKFYADSVKGRFTISRDNSKNTLFLQMNSLRAEDTAVYYCARDRILGAGFDYWGQGTLVTVSS

SEQ ID NO: 109 - protein, 119 aa, synthetic construct
  REGION 1..119: note = PR005509 VH
QVQLVESGGGVVQPGRSLRLSCAASGFTFSSYGFHWVRQAPGKGLEWLGLIWSDGSNKYYADSVKGRFTISRDNSKNTLYLQMNSLRAEDTAVYYCARDRIVGAGFDYWGQGTLVTVSS

SEQ ID NO: 110 - protein, 119 aa, synthetic construct
  REGION 1..119: note = PR005512 VH
QVQLVESGGGVVQPGRSLRLSCVASGFTFSSYGMHWVRQAPGKGLEWVAIIWNDGSNRYYADSVKGRFTISRDNSKNTLYLQMNSLRAEDTAVYYCARDRIVGAGFDYWGQGTLVTVSS

SEQ ID NO: 111 - protein, 119 aa, synthetic construct
  REGION 1..119: note = PR005514 VH, PR006839 VH
QVQLVESGGGVVQPGRSLRLSCAASGFTFSSYGFHWVRQAPGKGLEWLGLIWSDGSNKYYADSVKGRFTISRDNSKNTLYLQMNSLRAEDTAVYYCARDRIVGEGFDYWGQGTLVTVSS

SEQ ID NO: 112 - protein, 120 aa, synthetic construct
  REGION 1..120: note = PR006207 VH
QVQLVQSGAEVKKPGASVKVSCKASGFSFSSGYYMAWVRQAPGQGLEWMGLIGVGSGSLWYAQKFQGRVTMTRDTSTSTVYMELSSLRSEDTAVYYCARHFALSDPFNLWGQGTLVTVSS

SEQ ID NO: 113 - protein, 126 aa, synthetic construct
  REGION 1..126: note = PR006475 VH
QVQLQESGPGLVKPSGTLSLTCAVSGASISSGHWWSWVRQPPGKGLEWIGEIYHSGSTNYTPSLKSRVTISVDKSKNQFSLKLTSVTAADTAVYYCAREASYHYGSGSYYTPFDFWGQGTLVTVSS

SEQ ID NO: 114 - protein, 116 aa, synthetic construct
  REGION 1..116: note = PR006480 VH, PR006895 VH
QVQLVQSGGGVVQPGRSLRLSCAASGFTFSSYGMHWVRQAPGKGLEWVALIWYDGSNKYYADSVKGRFTISRDNSKNTLYLQMNSLRAEDTAVYYCARSVLLFDSWGQGTLVTVSS

SEQ ID NO: 115 - protein, 116 aa, synthetic construct
  REGION 1..116: note = PR007221 VH, PR007442 VH
QVQLVQSGGGVVQPGRSLRLSCAASGFTFSSYGMHWVRQAPGKGLEWVALIWYDASYKYYADSVKGRFTISRDNSKNTLYLQMNSLRAEDTAVYYCARSVLLFDSWGQGTLVTVSS

SEQ ID NO: 116 - protein, 116 aa, synthetic construct
  REGION 1..116: note = PR007223 VH, PR007444 VH
QVQLVQSGGGVVQPGRSLRLSCAASGFTFSSYGMHWVRQAPGKGLEWVALIWYDASNKYYADSVKGRFTISRDNSKNTLYLQMNSLRAEDTAVYYCARSVLLFNMWGQGTLVTVSS

SEQ ID NO: 117 - protein, 119 aa, synthetic construct
  REGION 1..119: note = PR007630 VH, PR007973 VH
QVQLVESGGGVVQPGRSLRLSCAASGFTFSSYGFHWVRQAPGKGLEWLGIIWSDASNKYYADSVKGRFTISRDNSKNTLYLQMNSLRAEDTAVYYCARDRIVGEGFDYWGQGTLVTVSS

SEQ ID NO: 118 - protein, 119 aa, synthetic construct
  REGION 1..119: note = PR007631 VH
QVQLVESGGGVVQPGRSLRLSCAASGFTFSSYGFHWVRQAPGKGLEWLGLIWSDARHKDYADSVKGRFTISRDNSKNTLYLQMNSLRAEDTAVYYCARDRIVGEGFDYWGQGTLVTVSS

SEQ ID NO: 119 - protein, 119 aa, synthetic construct
  REGION 1..119: note = PR007972 VH
QVQLVESGGGVVQPGRSLRLSCAASGFTFSYYGFHWVRQAPGKGLEWLGIIWSDASNKYYADSVKGRFTISRDNSKNTLYLQMNSLRAEDTAVYYCARDRIVGEGFDYWGQGTLVTVSS

SEQ ID NO: 120 - protein, 119 aa, synthetic construct
  REGION 1..119: note = PR007974 VH
QVQLVESGGGVVQPGRSLRLSCAASGFTFSYYGFHWVRQAPGKGLEWLGLIWSDARHKDYADSVKGRFTISRDNSKNTLYLQMNSLRAEDTAVYYCARDRIVGEGFDYWGQGTLVTVSS

SEQ ID NO: 121 - protein, 107 aa, synthetic construct
  REGION 1..107: note = PR002903 VL
DIQMTQSPSSLSASIGDRVTITCKASQDINSYLSWFQQKPGKAPKLLIYRANRLVDGVPSRFSGSGSGTDYTLTISSLQPEDFAVYYCLQYDEFPYTFGGGTKLEIK

SEQ ID NO: 122 - protein, 107 aa, synthetic construct
  REGION 1..107: note = PR002906 VL
DIQMTQSPSSLSASVGDRVTITCKASKNIRSYLAWYQQKPGKAPKLLIYSGSTLHSGVPSRFSGSGSGTDFTLTISSLQPEDFATYYCQQHHEYPLTFGQGTKVEIK

SEQ ID NO: 123 - protein, 112 aa, synthetic construct
  REGION 1..112: note = PR005474 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 124 - protein, 112 aa, synthetic construct
  REGION 1..112: note = PR005509 VL, PR005514 VL, PR006839 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTMYTFGQGTKLEIK

SEQ ID NO: 125 - protein, 112 aa, synthetic construct
  REGION 1..112: note = PR005512 VL
DIVMTQSPLSLPVTPGEPASISCRSSQSLLHSNGYNYLDWYLQKPGQSPQLLIYLGSNRASGVPDRFSGSGSGTDFTLKISRLEAEDVGVYYCMQGLQTPWTFGQGTKVEIK

SEQ ID NO: 126 - protein, 108 aa, synthetic construct
  REGION 1..108: note = PR006207 VL
EIVLTQSPDFQSVTPKEKVTITCQASESIDSYLLWYQQKPDQSPKLLIKQASTLASGVPSRFSGSGSGTDFTLTINSLEAEDAATYYCQNYYDISSNDFGGGTKVEIK

SEQ ID NO: 127 - protein, 106 aa, synthetic construct
  REGION 1..106: note = PR006475 VL
EIVLTQSPATLSVSPGERATLSCRASQSVSSNLAWYQQKPGQAPRLLIYGASTRATGIPARFSGSGSGTEFTLTISSLQSEDFALYYCQQYNNWIIFGQGTRLEIK

SEQ ID NO: 128 - protein, 107 aa, synthetic construct
  REGION 1..107: note = PR006480 VL, PR006895 VL
DIQMTQSPPSLSASVGDRVTITCRASQSISSYLNWFQQKAGKAPKLLISATSNLQSGVPSRFSGSGSGTDFTLTISSLQPEDLATYYCQQSYSSPYTFGQGTKVEIK

SEQ ID NO: 129 - protein, 107 aa, synthetic construct
  REGION 1..107: note = PR007221 VL
DIQLTQSPPSLSASVGDRVTITCRASQSISSYLNWFQQKAGKAPKLLISATSNLQSGVPSRFSGSGSGTDFTLTISSLQPEDLATYYCQQTLSSPYTFGQGTKVEIK

SEQ ID NO: 130 - protein, 107 aa, synthetic construct
  REGION 1..107: note = PR007223 VL
DIQLTQSPPSLSASVGDRVTITCRASQSISSYLNWFQQKAGKAPKLLISATSNLQSGVPSRFSGSGSGTDFTLTISSLQPEDLATYYCQQTYSSPYTFGQGTKVEIK

SEQ ID NO: 131 - protein, 107 aa, synthetic construct
  REGION 1..107: note = PR007442 VL
DIQMTQSPPSLSASVGDRVTITCRASQSISSYLNWFQQKAGKAPKLLISATSNLQSGVPSRFSGSGSGTDFTLTISSLQPEDLATYYCQQTLSSPYTFGQGTKVEIK

SEQ ID NO: 132 - protein, 107 aa, synthetic construct
  REGION 1..107: note = PR007444 VL
DIQMTQSPPSLSASVGDRVTITCRASQSISSYLNWFQQKAGKAPKLLISATSNLQSGVPSRFSGSGSGTDFTLTISSLQPEDLATYYCQQTYSSPYTFGQGTKVEIK

SEQ ID NO: 133 - protein, 112 aa, synthetic construct
  REGION 1..112: note = PR007630 VL
DIVMTQSPLSLPVTPGEPASISCRSSQSLLHRSGYNYLDWYLQKPGQSPQLLIYLGSYRASGVPDRFSGSGSGTDFTLKISRVEAEDVGVYYCMQGLQTMYTFGQGTKLEIK

SEQ ID NO: 134 - protein, 112 aa, synthetic construct
  REGION 1..112: note = PR007631 VL
DIVMTQSPLSLPVTPGEPASISCRSSQSLLHRSGYNYLDWYLQKPGQSPQLLIYLGSYRWSGVPDRFSGSGSGTDFTLKISRVEAEDVGVYYCMQGLQTMYTFGQGTKLEIK

SEQ ID NO: 135 - protein, 112 aa, synthetic construct
  REGION 1..112: note = PR007972 VL
DIVMTQSPLSLPVTPGEPASISCRSSQSLLHRNGYNYLDWYLQKPGQSPQLLIYLGSNRASGVPDRFSGSGSGTDFTLKISRVEAEDVGVYYCQQGIQTEYTFGQGTKLEIK

SEQ ID NO: 136 - protein, 112 aa, synthetic construct
  REGION 1..112: note = PR007973 VL
DIVMTQSPLSLPVTPGEPASISCRSSQSLLHRNGYNYLDWYLQKPGQSPQLLIYLGSYRASGVPDRFSGSGSGTDFTLKISRVEAEDVGVYYCQQGIQTEYTFGQGTKLEIK

SEQ ID NO: 137 - protein, 112 aa, synthetic construct
  REGION 1..112: note = PR007974 VL
DIVMTQSPLSLPVTPGEPASISCRSSQSLLHRSGYNYLDWYLQKPGQSPQLLIYLGSYRASGVPDRFSGSGSGTDFTLKISRVEAEDVGVYYCQQGIQTTYTFGQGTKLEIK

SEQ ID NO: 138 - protein, 442 aa, synthetic construct
  REGION 1..442: note = PR002903 HC
QVQLQQSGSELKKPGASVKISCKASGYSFTDYIILWVRQNPGKGLEWIGHIDPYYGSSNYNLKFKGRVTITADQSTTTAYMELSSLRSEDTAVYYCGRSKRDYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 139 - protein, 454 aa, synthetic construct
  REGION 1..454: note = PR002906 HC
QVQLQESGPGLVKPSETLSLTCTVSGYTITSGYDWSWIRQPPGKGLEWIGYINYGGSTNYKPSLGSRVTISVDTSKNQFSLKLSSVTAADTAVYYCARYNEYKSYIYDWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 449 aa, synthetic construct
  REGION 1..449: note = PR005474 HC
QVQLVESGGGVVQPGRSLRLSCAASAFTFSSYGMHWVRQAPGKGLEWVALIWNDGSNKFYADSVKGRFTISRDNSKNTLFLQMNSLRAEDTAVYYCARDRILGAGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 449 aa, synthetic construct
  REGION 1..449: note = PR005509 HC
QVQLVESGGGVVQPGRSLRLSCAASGFTFSSYGFHWVRQAPGKGLEWLGLIWSDGSNKYYADSVKGRFTISRDNSKNTLYLQMNSLRAEDTAVYYCARDRIVGAGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 449 aa, synthetic construct
  REGION 1..449: note = PR005512 HC
QVQLVESGGGVVQPGRSLRLSCVASGFTFSSYGMHWVRQAPGKGLEWVAIIWNDGSNRYYADSVKGRFTISRDNSKNTLYLQMNSLRAEDTAVYYCARDRIVGAGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 449 aa, synthetic construct
  REGION 1..449: note = PR005514 HC
QVQLVESGGGVVQPGRSLRLSCAASGFTFSSYGFHWVRQAPGKGLEWLGLIWSDGSNKYYADSVKGRFTISRDNSKNTLYLQMNSLRAEDTAVYYCARDRIVGEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 450 aa, synthetic construct
  REGION 1..450: note = PR006207 HC
QVQLVQSGAEVKKPGASVKVSCKASGFSFSSGYYMAWVRQAPGQGLEWMGLIGVGSGSLWYAQKFQGRVTMTRDTSTSTVYMELSSLRSEDTAVYYCARHFALSDPFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 456 aa, synthetic construct
  REGION 1..456: note = PR006475 HC
QVQLQESGPGLVKPSGTLSLTCAVSGASISSGHWWSWVRQPPGKGLEWIGEIYHSGSTNYTPSLKSRVTISVDKSKNQFSLKLTSVTAADTAVYYCAREASYHYGSGSYYTPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 446 aa, synthetic construct
  REGION 1..446: note = PR006480 HC
QVQLVQSGGGVVQPGRSLRLSCAASGFTFSSYGMHWVRQAPGKGLEWVALIWYDGSNKYYADSVKGRFTISRDNSKNTLYLQMNSLRAEDTAVYYCARSVLLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 449 aa, synthetic construct
  REGION 1..449: note = PR006839 HC
QVQLVESGGGVVQPGRSLRLSCAASGFTFSSYGFHWVRQAPGKGLEWLGLIWSDGSNKYYADSVKGRFTISRDNSKNTLYLQMNSLRAEDTAVYYCARDRIVGEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 446 aa, synthetic construct
  REGION 1..446: note = PR006895 HC
QVQLVQSGGGVVQPGRSLRLSCAASGFTFSSYGMHWVRQAPGKGLEWVALIWYDGSNKYYADSVKGRFTISRDNSKNTLYLQMNSLRAEDTAVYYCARSVLLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 446 aa, synthetic construct
  REGION 1..446: note = PR007221 HC, PR007442 HC
QVQLVQSGGGVVQPGRSLRLSCAASGFTFSSYGMHWVRQAPGKGLEWVALIWYDASYKYYADSVKGRFTISRDNSKNTLYLQMNSLRAEDTAVYYCARSVLLFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 446 aa, synthetic construct
  REGION 1..446: note = PR007223 HC, PR007444 HC
QVQLVQSGGGVVQPGRSLRLSCAASGFTFSSYGMHWVRQAPGKGLEWVALIWYDASNKYYADSVKGRFTISRDNSKNTLYLQMNSLRAEDTAVYYCARSVLLFNMWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 449 aa, synthetic construct
  REGION 1..449: note = PR007630 HC, PR007973 HC
QVQLVESGGGVVQPGRSLRLSCAASGFTFSSYGFHWVRQAPGKGLEWLGIIWSDASNKYYADSVKGRFTISRDNSKNTLYLQMNSLRAEDTAVYYCARDRIVGEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 449 aa, synthetic construct
  REGION 1..449: note = PR007631 HC
QVQLVESGGGVVQPGRSLRLSCAASGFTFSSYGFHWVRQAPGKGLEWLGLIWSDARHKDYADSVKGRFTISRDNSKNTLYLQMNSLRAEDTAVYYCARDRIVGEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 449 aa, synthetic construct
  REGION 1..449: note = PR007972 HC
QVQLVESGGGVVQPGRSLRLSCAASGFTFSYYGFHWVRQAPGKGLEWLGIIWSDASNKYYADSVKGRFTISRDNSKNTLYLQMNSLRAEDTAVYYCARDRIVGEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 449 aa, synthetic construct
  REGION 1..449: note = PR007974 HC
QVQLVESGGGVVQPGRSLRLSCAASGFTFSYYGFHWVRQAPGKGLEWLGLIWSDARHKDYADSVKGRFTISRDNSKNTLYLQMNSLRAEDTAVYYCARDRIVGEGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 214 aa, synthetic construct
  REGION 1..214: note = PR002903 LC
DIQMTQSPSSLSASIGDRVTITCKASQDINSYLSWFQQKPGKAPKLLIYRANRLVDGVPSRFSGSGSGTDYTLTISSLQPEDFAVYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 214 aa, synthetic construct
  REGION 1..214: note = PR002906 LC
DIQMTQSPSSLSASVGDRVTITCKASKNIRSYLAWYQQKPGKAPKLLIYSGSTLHSGVPSRFSGSGSGTDFTLTISSLQPEDFATYYCQQHHEYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 219 aa, synthetic construct
  REGION 1..219: note = PR005474 LC
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 219 aa, synthetic construct
  REGION 1..219: note = PR005509 LC, PR005514 LC, PR006839 LC
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 219 aa, synthetic construct
  REGION 1..219: note = PR005512 LC
DIVMTQSPLSLPVTPGEPASISCRSSQSLLHSNGYNYLDWYLQKPGQSPQLLIYLGSNRASGVPDRFSGSGSGTDFTLKISRLEAEDVGVYYCMQGLQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 215 aa, synthetic construct
  REGION 1..215: note = PR006207 LC
EIVLTQSPDFQSVTPKEKVTITCQASESIDSYLLWYQQKPDQSPKLLIKQASTLASGVPSRFSGSGSGTDFTLTINSLEAEDAATYYCQNYYDISSNDFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 213 aa, synthetic construct
  REGION 1..213: note = PR006475 LC
EIVLTQSPATLSVSPGERATLSCRASQSVSSNLAWYQQKPGQAPRLLIYGASTRATGIPARFSGSGSGTEFTLTISSLQSEDFALYYCQQYNNWIIFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 214 aa, synthetic construct
  REGION 1..214: note = PR006480 LC, PR006895 LC
DIQMTQSPPSLSASVGDRVTITCRASQSISSYLNWFQQKAGKAPKLLISATSNLQSGVPSRFSGSGSGTDFTLTISSLQPEDLATYYCQQSYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 214 aa, synthetic construct
  REGION 1..214: note = PR007221 LC
DIQLTQSPPSLSASVGDRVTITCRASQSISSYLNWFQQKAGKAPKLLISATSNLQSGVPSRFSGSGSGTDFTLTISSLQPEDLATYYCQQTLSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 214 aa, synthetic construct
  REGION 1..214: note = PR007223 LC
DIQLTQSPPSLSASVGDRVTITCRASQSISSYLNWFQQKAGKAPKLLISATSNLQSGVPSRFSGSGSGTDFTLTISSLQPEDLATYYCQQTYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 214 aa, synthetic construct
  REGION 1..214: note = PR007442 LC
DIQMTQSPPSLSASVGDRVTITCRASQSISSYLNWFQQKAGKAPKLLISATSNLQSGVPSRFSGSGSGTDFTLTISSLQPEDLATYYCQQTLSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 214 aa, synthetic construct
  REGION 1..214: note = PR007444 LC
DIQMTQSPPSLSASVGDRVTITCRASQSISSYLNWFQQKAGKAPKLLISATSNLQSGVPSRFSGSGSGTDFTLTISSLQPEDLATYYCQQTYSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 219 aa, synthetic construct
  REGION 1..219: note = PR007630 LC
DIVMTQSPLSLPVTPGEPASISCRSSQSLLHRSGYNYLDWYLQKPGQSPQLLIYLGSYRASGVPDRFSGSGSGTDFTLKISRVEAEDVGVYYCMQGLQTMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 219 aa, synthetic construct
  REGION 1..219: note = PR007631 LC
DIVMTQSPLSLPVTPGEPASISCRSSQSLLHRSGYNYLDWYLQKPGQSPQLLIYLGSYRWSGVPDRFSGSGSGTDFTLKISRVEAEDVGVYYCMQGLQTMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 219 aa, synthetic construct
  REGION 1..219: note = PR007972 LC
DIVMTQSPLSLPVTPGEPASISCRSSQSLLHRNGYNYLDWYLQKPGQSPQLLIYLGSNRASGVPDRFSGSGSGTDFTLKISRVEAEDVGVYYCQQGIQTEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 219 aa, synthetic construct
  REGION 1..219: note = PR007973 LC
DIVMTQSPLSLPVTPGEPASISCRSSQSLLHRNGYNYLDWYLQKPGQSPQLLIYLGSYRASGVPDRFSGSGSGTDFTLKISRVEAEDVGVYYCQQGIQTEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 219 aa, synthetic construct
  REGION 1..219: note = PR007974 LC
DIVMTQSPLSLPVTPGEPASISCRSSQSLLHRSGYNYLDWYLQKPGQSPQLLIYLGSYRASGVPDRFSGSGSGTDFTLKISRVEAEDVGVYYCQQGIQTTYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 348 aa, Homo sapiens
MLCPWRTANLGLLLILTIFLVAEAEGAAQPNNSLMLQTSKENHALASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLYIPYIILTIIILTIVGFIWLLKVNGCRKYKLNKTESTPVVEEDEMQPYASYTEKNNPLYDTTNKVKASEALQSEVDTDLHTL

SEQ ID NO: 173 - protein, 325 aa, Homo sapiens
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLYIPYIILTIIILTIVGFIWLLKVNGCRKYKLNKTESTPVVEEDEMQPYASYTEKNNPLYDTTNKVKASEALQSEVDTDLHTL

SEQ ID NO: 174 - protein, 271 aa, Homo sapiens
MSAPRLLISIIIMVSASSSSCMGGKQMTQNYSTIFAEGNISQPVLMDINAVLCCPPIALRNLIIITWEIILRGQPSCTKAYKKETNETKETNCTVERITWVSRPDQNSDLQIRPVDTTHDGYYRGIVVTPDGNFHRGYHLQVLVTPEVNLFQSRNITAVCKAVTGKPAAQISWIPEGSILATKQEYWGNGTVTVKSTCPWEGHKSTVTCHVSHLTGNKSLSVKLNSGLRTSGSPALSLLIILYVKLSLFVVILVTTGFVFFQRINHVRKVL

SEQ ID NO: 175 - protein, 349 aa, Macaca mulatta
MLCPWRTANLGLLLILAVFLVAEAEGAAQSNNSLMLQTSKENHTLASNSLCMDEKQITQNHSKVLAEVNISWPVQMARNAVLCCPPIEFRNLIVITWEIILRGQPSCTKTYRKDTNETKETNCTDERITWVSTPDQNSDLQIHPVAITHDGYYRCIMATPDGNFHRGYHLQVLVTPEVTLFESRNRTAVCKAVAGKPAAQISWIPAGDCAPTEQEYWGNGTVTVKSTCHWEGHNVSTVTCHVSHLTGNKSLYIELLPVPGAKKSAKLYMPYVILTIIILTIVGFIWLLKISGCRKYNLNKTESTSVVEEDEMQPYASYTEKNNPLYDTTNKVKASQALQSEVGTDLHTL

SEQ ID NO: 176 - protein, 326 aa, Macaca fascicularis
MLCPWRTANLGLLLILAVFLVAASNSLCMDEKQITQNHSKVLAEVNISWPVQMARNAVLCCPPIEFRNLIVITWEIILRGQPSCTKTYRKDTNETKETNCTDERITWVSTPDQNSDLQIHPVAITHDGYYRCIMATPDGNFHRGYHLQVLVTPEVTLFESRNRTAVCKAVAGKPAAQISWIPAGDCAPTEHEYWGNGTVTVKSTCHWEGHNVSTVTCHVSHLTGNKSLYIELLPVPGAKKSAKLYMPYVILTIIILTIVGFIWLLKISGCRKYNLNKTESTSVVEEDEMQPYASYTEKNNPLYDTTNKVKASQALQSEVGTDLHTL

SEQ ID NO: 177 - protein, 275 aa, Macaca fascicularis
MHTLGKMSASRLLISIIIMVSASSSSCMDGKQMTQNYSKMSAEGNISQPVLMDTNAMLCCPPIEFRNLIVIVWEIIIRGQPSCTKPYRKETNETKETNCTDERITWVSTPDQNSDLQIHPVAITHDGYYRCIMATPDGNFHRGYHLQVLVTPEVTLFQSRNRTAVCKAVAGKPAAQISWIPAGDCAPTEHEYWGNGTVTVESMCHWGDHNASTVTCHVSHLTGNKSLYIKLNSGLRTSGSPALDLLIILYVKLSLFVVILVTTGFVFFQRINYVR

SEQ ID NO: 178 - protein, 480 aa, synthetic construct
  REGION 1..480: note = hCD200R1-Fc sequence
EGAAQPNNSLMLQTSKENHALASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVPGAKKSAKLMVTVSSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 447 aa, synthetic construct
  REGION 1..447: note = PR200526 HC
QVQLVQSGAEVKKPGQSLRISCKGAGYSFSTYWIGWVRQMPGKGLEWMGIIYPDDSDTRYSPSFEGQVTISVDKSITTAYLHWSSLKASDTAIYYCARLVGGAPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 216 aa, synthetic construct
  REGION 1..216: note = PR200526 LC
QSALTQPASVSGSPGQSITISCSGSSSDIGRYDYVSWYQHYPDKAPKLIIYEVKHRPSGISHRFSASKSGNTASLTISELQPGDEADYYCASYTESKTYIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 181 - protein, 453 aa, synthetic construct
  REGION 1..453: note = PR200691 HC
QVQLVESGGGVVQPGRSLRLSCAASGFTFSRYGMHWVRQAPGKGLEWVAVIPYDGSNKYYADSVKGRFTISRDISKNTLYLQMNSLRAEDTAVYYCARRGYYDSSGYYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 182 - protein, 215 aa, synthetic construct
  REGION 1..215: note = PR200691 LC
EIVMTQSPATLSVSPGERATLSCRASQSVSSNLAWYQQKPGQAPRLLIYGASTRATGIPARFSGSGSGTEFTLTISSLQSEDFAVYYCQQYNKWPPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 447 aa, synthetic construct
  REGION 1..447: note = PR000150 HC
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 184 - protein, 218 aa, synthetic construct
  REGION 1..218: note = PR000150 LC
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 269 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDREP